(19) United States
(12) Patent Application Publication
(10) Pub. No.: US 2021/0228741 A1
(43) Pub. Date: Jul. 29, 2021

(54) METHODS AND COMPOSITIONS TO STIMULATE RETINAL REGENERATION

(71) Applicant: UNIVERSITY OF WASHINGTON, SEATTLE, WA (US)

(72) Inventors: Thomas A. REH, SEATTLE, WA (US); Nikolas L. JORSTAD, SEATTLE, WA (US); Stefanie G. WOHL, SEATTLE, WA (US)

(73) Assignee: UNIVERSITY OF WASHINGTON, SEATTLE, WA (US)

(21) Appl. No.: 17/050,999

(22) PCT Filed: Apr. 29, 2019
(86) PCT No.: PCT/US19/29725

§ 371 Date: Oct. 27, 2020

Related U.S. Application Data
(60) Provisional application No. 62/663,589, filed on Apr. 27, 2018.

(51) Int. Cl. A61K 48/00; C07K 14/47; C12N 9/80; C12N 15/113; A61K 9/00; A61P 27/02

(52) U.S. Cl. CPC A61K 48/0058; C07K 14/4705; C12N 9/80; C12Y 305/01098; C12N 2310/141; A61K 48/0075; A61K 9/0048; A61P 27/02; C12N 15/113

(57) ABSTRACT

Methods and compositions are provided for regeneration of retinal neurons and vision restoration after injury, disease, or loss comprising, administration of a nucleic acid sequence encoding Ascl1 and retinal reprogramming potentiating agents, including HDAC inhibitors, STAT-signaling inhibitors and microRNA manipulations in Ascl1-induced retinal regeneration.

### ACKNOWLEDGEMENT OF GOVERNMENT SUPPORT

This invention was made with Government support under R01 EY021482, awarded by the National Institutes of Health. The Government has certain rights in the invention.

This application claims benefit of U.S. provisional patent application No. 62/663,589, filed Apr. 27, 2018, the entire contents of which are incorporated by reference into this application.

### REFERENCE TO A SEQUENCE LISTING SUBMITTED VIA EFS-WEB

The content of the ASCII text file of the sequence listing named “UW68WOU1_seq” which is 81 kb in size was created on Apr. 29, 2019, and electronically submitted via EFS-Web herewith the application is incorporated herein by reference in its entirety.

### BACKGROUND OF THE INVENTION

Some of the leading causes of blindness involve the loss of one or more types of retinal neurons. Age-related macular degeneration, glaucoma, ischemia, retinal arterial occlusion and inherited retinal diseases, such as Retinitis Pigmentosa or Usher's syndrome all ultimately lead to irreversible degeneration of photoreceptors or other retinal neurons. In mammalian retinas, lost neurons are not spontaneously regenerated, and currently, there are no effective therapies to replace the degenerated neurons in patients with retinal disease. By contrast, retinas of nonmammalian vertebrates, such as fish and amphibians, show a robust regenerative response upon retinal damage. Upon injury to the retina, fish Müller Glia (MG) re-enter the cell cycle to generate a progenitor, which proliferates, and generates different types of retinal neurons to replace those that were lost.

One key difference between fish and mouse MG in the response to retinal injury is the expression of the proneural transcription factor, Ascl1. This factor is necessary for regeneration in fish retina, but is not expressed in mammalian MG after injury. When we over-expressed Ascl1 in mouse MG with a lentivirus, we found that this single factor could reprogram them into neurogenic progenitors in vitro. When Ascl1 expression is induced in adult MG, the combination of Ascl1 and histone deacetylase (HDAC) inhibition can stimulate new neuron production from MG in adult mice after neuronal damage. The MG-derived neurons form connections with the existing retinal circuitry. Patch-clamp recordings from MG-derived neurons in retinal slices shows that they have neuronal-like light responses. Epigenetic analyses show that chromatin remodelers can produce a neurogenic potential to adult MG by making previously inaccessible neuronal genes open to proneural transcription factors, like Ascl1. In addition, a specific micro-RNA, miR-124, can induce expression of Ascl1 in Müller glial cells, and reprogram them to a neurogenic state, resulting in neuron production. Only a small percentage of MG are reprogrammed using these previous methods, however, limiting their promise for therapeutic applications.

There remains a need to stimulate regeneration in human retina for the development of new types of regenerative therapies for patients.

### SUMMARY OF THE INVENTION

Described herein are compositions, nucleic acid molecules, and methods for inducing retinal regeneration in a subject.

Disclosed herein is a nucleic acid molecule comprising a nucleic acid sequence encoding Ascl1 and a nucleic acid sequence encoding one or more reprogramming potentiators.

In one embodiment, the one or more reprogramming potentiators is selected from the group consisting of one or more HDACi, one or more Jak/STATi, one or more Ascl1 activators, and a combination thereof. In another embodiment, the one or more HDACi is selected from the group consisting of 16cyc-HxA, 161lin-HxA, 16KA, and a combination thereof. In another embodiment, the one or more Jak/STATi is selected from the group consisting of Socs1, Socs2, Socs3, Socs4, Socs5, Socs6, Socs7, CIS, XpYL and a combination thereof. In another embodiment, the one or more Ascl1 activators is selected from the group consisting of miR-25, miR-124, one or more let7 family inhibitors, and a combination thereof.

Also disclosed herein is a method for inducing retinal regeneration in a subject comprising: a) administering to a retina of the subject a nucleic acid molecule comprising a nucleic acid sequence encoding Ascl1 and a nucleic acid sequence encoding one or more reprogramming potentiators, wherein Ascl1 induces neurogenesis from MG, and wherein the one or more reprogramming potentiators stimulate production of functional neurons from the Ascl1-induced MG. In one embodiment, the number of the MG-derived functional neurons is increased. In another embodiment, the number of functional neurons is increased by 40%. In another embodiment, the subject is treated for retinal disease, damage or degeneration in the retina. In another embodiment, the subject is an adult. In another embodiment, a vector comprises the nucleic acid molecule. In one embodiment, the vector is a non-viral vector or a viral vector, and the viral vector is an adeno-associated viral (AAV) vector or a lentiviral vector. In an additional embodiment, a promoter sequence is in operable linkage with the nucleic acid encoding Ascl1. In one embodiment, the promoter is a retinal or MG-specific promoter. In one embodiment, administering to the retina is intravitreal or subretinal injection. In another embodiment, the one or more reprogramming potentiating agents are selected from the group consisting of 1) miR-25, or an activator or mimic thereof; 2) miR-124, or an activator or mimic thereof; 3) one or more let-7 family inhibitors or antagomirs; and 4) a combination thereof. In another embodiment, miR-25, miR-124, or the one or more let7 are expressed by a short hairpin RNA (shRNA).

Also disclosed herein is a method for inducing retinal regeneration in a subject comprising: a) administering to a retina of the subject a polynucleotide sequence encoding Ascl1 (Achaete-scute-like family bHLH transcription factor 1), wherein expression of Ascl1 induces neurogenesis from Müller glia (MG); and b) sequentially or concurrently administering to the retina of the subject a combined therapy comprising a composition comprising one or more TSA/HDAC inhibitors (HDACi): and a composition comprising one or more Jak/STAT signaling pathway inhibitors (STATi), wherein the combined therapy stimulates production of functional neurons from the Ascl1-induced neurogenesis from the MG.

Additionally disclosed herein is a nucleic acid molecule comprising a nucleic acid sequence encoding Ascl1 and an MG-specific promoter sequence. In one embodiment, the MG-specific promoter sequence is a Rbpl1 promoter sequence or a portion thereof.

Also disclosed herein is a nucleic acid molecule comprising a nucleic acid sequence encoding Ascl1 and a shRNA. In one embodiment, the shRNA is a miR-25, miR-124, or let-7 shRNA.

In some embodiments, the method comprises (a) administering to the subject a composition comprising one or more Ascl1 (Achaete-scute-like 1) gene expression activators; (b) administering to the subject a composition comprising one or more histone deacetylase inhibitors (HDACi); and (c) administering to the subject a composition comprising a reprogramming potentiating agent. In some embodiments, the reprogramming potentiating agent is: (i) one or more Signal Transducer and Activator of Transcription (STAT) signaling pathway inhibitors; and/or (ii) microRNA miR-25 (hereinafter “miR-25”); and one or more let-7 family inhibitors. In some embodiments, the composition for retinal regeneration comprises (a) one or more miR-25 activators or mimics thereof; (b) one or more microRNA 124 (miR-124) activators or mimics thereof; and (c) one or more let-7 family inhibitors or antagomirs. In some embodiments, the one or more Ascl1 gene expression activators, the one or more HDACi; the one or more STAT signaling pathway inhibitors; the miR-25; and the one or more let-7 family inhibitors are administered sequentially. In some embodiments, the one or more Ascl1 gene expression activators, the one or more HDACi; the one or more STAT signaling pathway inhibitors; the miR-25; and the one or more let-7 family inhibitors are administered concurrently.

In one embodiment, the composition administered in step (c) comprises one or more STAT signaling pathway inhibitors. In another embodiment, the compositions administered in step (c) comprise microRNA miR-25; and one or more let-7 family inhibitors. In a further embodiment, the compositions administered in step (c) comprise each of one or more STAT signaling pathway inhibitors; miR-25; and one or more let-7 family inhibitors.

Also provided are compositions for retinal regeneration. In some embodiments, the composition comprises (a) one or more Ascl1 gene expression activators; (b) one or more HDACi inhibitors; and (c) one or more reprogramming potentiating agents. The reprogramming potentiating agents, in some embodiments, are selected from (i) one or more STAT signaling pathway inhibitors: (ii) miR-25; and (iii) one or more let-7 family inhibitors.

In one embodiment of the composition, the agents of (c) comprise one or more STAT signaling pathway inhibitors. In another embodiment, the agents of (c) comprise miR-25; and one or more let-7 family inhibitors. In some embodiments, the agents of (c) comprise one or more STAT signaling pathway inhibitors; miR-25; and one or more let-7 family inhibitors. Representative examples of the HDACi include, but are not limited to, trichostatin A (TSA), Istodax™ also known as (Pro)/romidepsin, Beleodaq™, also known as (Pro)/belinostat, Farydak™, also known as (Pro)/panobinostat, and Zolinza™, also known as (Pro)/vorinostat. In one embodiment, the STAT signaling pathway inhibitor is selected from the group consisting of peptidomimetics, small molecule inhibitors and oligonucleotides. Examples of endogenous STAT pathway inhibitors, include, but are not limited to, suppressor of cytokine signaling (SOCS) proteins, phosphatases, and protein inhibitor of activated STAT (PIAS) proteins. Such endogenous inhibitors provide a basis for therapeutic molecules and compounds for STAT inhibition. In one embodiment, the STAT signaling pathway inhibitor is an inhibitor of STAT3. Examples of STAT3 inhibitors include, but are not limited to, SH-4-54. See also Fagard et al. JAKSTAT. 2013 Jan. 1; 2(1): e22882.

Also provided herein are methods for inducing retinal regeneration comprising administering to a subject a composition as described herein. In some embodiments, the methods are effective to increase the number of Müller glial-derived neurons, to induce Müller glial cells to enter the mitotic cell cycle, and/or to generate new retinal neurons. In some embodiments, the new retinal neurons are bipolar neurons. In some embodiments of the method, the number of retinal neurons increases by at least 40% relative to a baseline level or other reference amount representative of an untreated retina. In some embodiments, the number of retinal neurons increases by 10%, 20%, 25%, 50%, 100%, 150%, 200%, or more.

The subject is typically a mammal, such as a human or veterinary subject. In one embodiment, the subject is an adult. The subject, in some embodiments, has a retinal degenerative disease. Examples of such retinal degenerative diseases include, but are not limited to, Age-related macular degeneration, glaucoma, ischemia, retinal arterial occlusion and inherited retinal diseases, such as Retinitis Pigmentosa or Usher's syndrome.

### BRIEF DESCRIPTION OF THE DRAWINGS

FIG. 1 shows that STAT pathway inhibition increases the number of Müller glial-derived neurons. A) Experimental paradigm for increasing MG-derived regeneration efficiency. Tamoxifen is administered for up to 5 consecutive days, followed by NMDA damage a few days after tamoxifen, followed by administration of TSA and/or STAT inhibition a couple days after damage. Retinas were collected a minimum of two weeks after TSA/STATi. B) Representative image showing ANT-treated adult retina with MG-derived neurons. C) Representative image showing ANTSi-treated adult retina with increased number of MG-derived neurons. D) Shows enlargement of ANTSi-treated retinas from C. Arrows indicate Cabp5+Otx2+GFP+cells. All images are flattened Z-stacks. Scale bars for B-C are 20 μm. ONL=Outer Nuclear Layer, OPL=Outer Plexiform Layer, INL=Inner Nuclear Layer. IPL=Inner Plexiform Layer, GCL=Ganglion Cell Layer. E) Quantification of Otx2 in ANT (n=16) and ANTSi-treated (n=13) retinas. F) Quantification of Cabp5 in ANT (n=6) and ANTSi-treated (n=8) retinas. ANT vs ANTSi treatments in E and F were significantly different by unpaired t-test at **P=0.0023 and ***P=0.0006, respectively. G) Experimental paradigm for testing where ANTSi-treated MG proliferate prior to neurogenesis. H) Representative image from proliferation experiment showing EdU and GFP colocalization. 1) Shows enlargement of H and highlights GFP+EdU+Cabp5+MG-derived neurons (arrows). Scale bars for H-I are 50 μm and 20 μm, respectively.

FIG. 2 shows that the STAT inhibitor SH-4-54 inhibits pSTAT3 in MG. A) Experimental paradigm for analyzing STAT3 phosphorylation by western blot in MG. P11 retinas were dissociated and grown in a growth media containing mEGF for 7 DIV. MG were passaged to eliminate residual neurons and cultured in a media containing doxycycline to induce Ascl1 expression for 6 DIV. On the sixth day, the STATi SH-4-54 was added, then cells were harvested 24 hours later for WB. B) Representative images from confluent cultures of non-treated, DOX-treated, and DOX+STATi-treated MG just before harvesting. C) Representative western blot for pSTAT3 (Y705), STAT3, and Beta-actin on non-treated, Dox-treated, and Dox+STATi-treated MG cultures. D) Graph showing quantification of western blot bands was significantly different by unpaired t-test, **P=0.0029. E) Representative image of WT retinas injected with either PBS, NMDA, or SH-4-54 and collected 24 hours later for TUNEL staining. Scale bars for B and E are 25 and 40 μm, respectively.

FIG. 3 shows that ANTSi-treated MG-derived neurons (GFP appears lighter and brighter in grayscale images compared to OPN1SW) integrate into existing retinal circuits. A) and B) Example images of MG-derived neuron (lower, in INL) contacting cone photoreceptors (upper, in OPL and ONL) in the OPL, scale bars are 10 μm and 5 μm, respectively. C) Enlargement of B showing 0.18 μm z-stack steps of the MG-derived neurons making contact with cone pedicle, scale bar is 2 μm. D) Example image of MG-derived neuron with neuronal process in OPL and IPL, scale bar is 20 μm. E) Enlargement of arrow from D showing a Psd95+Ctbp2+photoreceptor synapse onto an apical MG-derived neuronal process in ONL. Left image sets show XY projection and right image sets show YZ projection, scale bar is 2 μm. F) Enlargement of arrow from D showing Ctbp2 staining within the MG-derived neuronal processes in IPL. Upper right image shows stringent GFP mask used to identify Ctbp2 puncta within MG-derived neurons, scale bar is 5 μm. G) Enlargement of arrowhead from F showing Ctbp2 within masked GFP process, directly apposed to Psd95 staining, consistent with synaptic specializations. Upper image sets show XY projection and lower image sets show XZ projection, scale bar is 0.5 μm. H) Population data for input resistance and visual responses recorded with the current-clamp technique. I) Population data for input resistance and resting membrane potential measurements. H and I, Solid lighter circles indicate new measurements recorded from ANTSi treatment condition and hollow lighter circles indicate measurements from ANT treatment condition from a previous study. Traces in H show maximum evoked light response from 500-ms luminance stimulation for MG (left) and MG-derived neuron (right). Traces in I show representative responses to equal increasing steps of injected current for MG (left) and MG-derived neuron (right). ANTSi-treated recordings were performed at 2, 5, and 7 weeks post-TSA and STATi administration.

FIG. 4 shows that ANTSi treatment results in more MG-derived neurons by scRNA-seq. A) A tSNE plot of FACS-purified WT MG and ANT-treated cells from our previous study¹⁶, and ANTSi-treated cells. B) Feature plots of glial (GluI), progenitor (DII1), neuronal (Cabp5 and Otx2), rod (Rho), and microglial (Aif1) gene expression to identify clusters by cell type. Lighter gray shows high-expressing, darker gray shows non-expressing cells. C) Plot from A shaded by treatment condition. D) Clusters pseudo colored in grayscale by cell type as determined by C. Microglia and rods made up contaminating populations of ˜5% and 2% of the total cells in each treatment group, respectively. E) Graph showing the fraction of each treatment that was comprised of MG, Progenitor-like cells, and MG-derived neurons. ANTSi treatment increases the population of MG-derived neurons and decreases the number of progenitor-like cells relative to ANT treatment. F) Heatmap showing all cells from all three treatments. MG-derived neurons express synaptic genes and do not express Stat pathway targets. Progenitor-like cells highly express Stat pathway targets. Scale shows log 2 expression.

FIG. 5 provides Pseudotime analysis of scRNA-seq datasets. A) Trajectory analysis in Monocle showing clusters from subset of data that includes MG, Progenitor-like cells, and MG-derived neurons. B) Trajectory analysis showing pseudotime progression, with cluster 3 (from A) being the root state. C, D) Gene expression of GluI, DII1, and Cabp5 shown as trajectory plot C, and pseudotime plot D.

FIG. 6 illustrates Ascl1 ChIP-seq from Ascl1-overexpressing MG and P0 retinal progenitors. A) Epigenetic analyses of progenitor genes DII1 and DII3. Tracks show biological replicate Ascl1 ChIP-seq peaks from P0 whole retina (P0 retina #1, #2), bars indicate peaks that were called from peak-calling algorithm HOMER with FDR of 0.1% (Retina Peaks track), peaks that were called from retinal, spinal cord, and NPC Ascl1 ChIP-seq datasets (Core Ascl1 track), and DNase-seq peaks from P0 whole retina showing accessible chromatin (P0 DNase track). Shaded highlights indicate core/common binding sites. Scale at the bottom track (X-axis=kilobases of genomic DNA, Y-axis=reads per million, RPM). B) Venn diagram showing proportions of overlap for Ascl1 ChIP-seq peaks between P0 retina, NPCs, and spinal cord (Left). Gene Ontology analysis of retinal-specific and core/common Ascl1 ChIP-seq peaks using GREAT algorithm and example genes in these categories (Right). C) Epigenetic comparison of Ascl1-overexpressing MG (MG Ascl1 and MG DNase tracks) with P0 developing retina (P0 Ascl1 and P0 DNase tracks). Additional tracks showing previously described Stat3 ChIP-seq peaks from brain oligodendrocytes (STAT ChIP) and comparative peak overlap analyses of Ascl1 peaks without DHSs (MG_{A_n_}MG_{D}) or with DHSs (MG_{A_e_}MG_{D}). D) Above, Venn diagram showing proportions of overlap of Ascl1 ChIP-seq peaks with DNase-seq peaks from Ascl1-overexpressing MG. Below, Pie chart showing the proportion of Ascl1 ChIP-seq peaks that have a P0 DNase peak present. E) Venn diagram showing proportions of overlap from Ascl1 ChIP-seq peaks between P0 developing retina and Ascl1-overexpressing MG (Top). Integrative analysis looking for motifs that were enriched at Ascl1-overexpressing MG-specific Ascl1 peaks (developmentally inappropriate), are located within ±5 kb of the T.S.S., and are associated with 0.75 increase in gene expression (from a previous Ascl1-virus vs GFP-virus microarray). Top scoring motifs meeting these criteria are presented in box (E-box 92%; SEQ ID NO: 33, Paired homebox 7%; SEQ ID NO: 34, Stat1/3/512%; SEQ ID NO: 35) (Below). F) Epigenetic comparison of Gadd45g, Id1, and Id3 gene loci in Ascl1-overexpressing MG (MG Ascl1) and P0 developing retina (P0 Ascl1). Additional tracks showing previously described Stat3 ChIP-seq peaks (STAT ChIP), and comparative peak overlap analyses of sites containing a MG Ascl1 peak, a P0 Ascl1 peak, and a Stat3 peak (MG_{A_e_}P0__{e_}STAT) or sites containing a MG Ascl1 peak, a Stat3 peak, but no P0 Ascl1 peak (MG_{A_n_}P0__{e_}STAT). Shaded highlights on right indicate strong Ascl1 binding sites during development and forced Ascl1 expression, darker shaded (left) highlights indicate anomalies.

FIG. 7 presents an analysis of P0 and MG Ascl1 ChIP-seq peaks. A) Motif enrichment analysis from MEME of top scoring motif from P0 Ascl1 ChIP-seq (2 replicates) and diagram showing central enrichment around the Ebox motif. B) Gene ontology analysis from GREAT showing top 5 enriched categories by −log 10 (binomial p value) for P0-specific peaks, Common peaks, and MG-specific peaks.

FIG. 8 demonstrates that Ascl1-overexpression results in dysregulated STAT-target genes. A) Schematic illustration of experimental paradigm for analyzing STAT-target genes in Ascl1-overexpressing MG. B) Both GFP−Sox2+ and Ascl1-expressing GFP+MG express Id1 2 days after NMDA damage. C) Ascl1+cells have higher expression of Id1 relative to GFP-MG (Sox2+cells) 4 days after NMDA damage. D) Id1 expression is reduced in GFP-MG but remains in the Ascl1-expressing GFP+MG 14 days after NMDA damage. Note, the flat Id1+nuclei in all images not labelled with GFP are endothelial cells. Scale bars for B-D are 20 μm. E) Graph showing RT-qPCR for Id1 gene expression relative to WT on retinas treated 4 days post-NMDA. One-way ANOVA with Tukey's post-test; *P<0.05, N=4 biological replicates per condition run in triplicate. F) Id1 expression is reduced in GFP-MG but remains in the Ascl1-expressing GFP+MG 14 days after ANT treatment (white arrows). MG-derived neurons (gray arrows) do not express Id1. Scale bar is 40 μm.

FIG. 9 demonstrates that microRNAs highly expressed in RPCs and MG. A) Heatmap of the results of a hierarchical clustering of the most highly expressed miRNAs across all ages in the RPCs (P2) and MG (P8, P11, Adult). B) Scatter plot of Log 2 counts of the miRNAs from RPCs and adult MG. All miRNAs expressed above the black line are miRNAs more highly expressed in RPCs than in MG, with the top progenitor miRNAs highlighted in gray. All miRNAs below the black line are miRNAs more highly expressed in MG than in RPCs with the top MG miRNAs highlighted in darker gray. C) Time course of 9 miRNAs that increase the most from P2 to adult (RPC-miRs). D) Time course of 11 miRNAs that are highly expressed in RPCs and decline during MG maturation.

FIG. 10 shows that overexpression of miR-25 and/or antagonism of let-7 induce Ascl1 expression in Müller glia. A) Schematic of the Ascl1CreER: tdTomatoflSTOP/flSTOP mouse B) Experimental design. C/C′-H/H′) Live images of tdTomato+cells after transfection with control (C,C′), RPC-miR cocktail (D,D′), miR-25 mimic (E,E′), let-7 antagomiR (AR, F,F′), miR-25 and let-7 AR (G,G′) or mir-25 and miR-124 mimic and let-7AR (H,H′), 4 days post transfection (dptf). I-K) Number of Ascl1tdTomato+cells per field 3-5 dptf with single or combined RPC-miR mimics (I, n≥3), single MG-miR antagomiRs (AR, J, n 2 3), or combined miR-25 mimics with let-7 AR with or without miR-124 mimic as well as miR-124 mimic alone (K, n 2 5). Scale bars 200 μm (C-H) and 100 μm (C′-H′). Significant differences of each treatment group from control wells are indicated: * p<0.05, ** p<0.01, Mann-Whitney-test, n: number of cultures of 6-8 mice per culture.

FIG. 11 illustrates the neuronal identity of the cells in the miR-treated conditions. A) miR-25/miR-124/let-7 reprogrammed Ascl1 expressing Müller glia express Map2 and Otx2. A) Ascl1-tdTomato reporter mouse. B) Experimental design. C/C′-F) Immunofluorescent labeling for tdTomato (AsclTom), Map2, and Otx2, as well as DAPI nuclear labeling, 7 days post transfection (7dptf). Map2+Otx2+neurons are either highly branched (arrowhead in F) or have one long process with a growth cone-like structure (arrows). G) Percentage of AsclTom+Map2+cells of total AsclTom+cells. 7 dptf (n=6). H) Percentage of AsclTom+Otx2+cells of total AsclTom+cells 7 dptf (n=4). I) Percentage of Map2+/Otx2+/Ascl1Tom+cells/Ascl1Tom+cells 7dptf (n=3). Scale bars 100 μm, in zoom ins 50 μm, in F 20 μm. Significant differences for each treatment condition from control wells are indicated*: p<0.05, ** p<0.01, ***: p<0.001 (For G, H) Mann-Whitney-test, for HI, Levene's test for variances and t-test), n: number of cultures of 6-8 mice per culture.

FIG. 12 shows that miR-25/miR-124/let-7 reprogrammed Rlbp1+Müller glia express TUJ and Otx2. A) Schematic of the Rlbp1CreER: tdTomatoflSTOP/flSTOP mouse. B) Experimental design. C/C′-G/G″) Immunofluorescent labeling for tdTomato (Rlbp1), TUJ1, and Otx2 or EdU (G-G″), as well as DAPI nuclear labeling, 6 days post transfection (dptf). The arrowhead in F/F′ shows Otx2-, the arrows Otx2+neurons. The lower left arrow in G-G″ shows a BrdU−the white upper right arrow a BrdU+neuron. H) Percentage of TUJ1+Rlbp:TdTomato+cells of total Rlbp:tdTomato+cells 3d (n=3) and 6 dptf, n=8). I) Percentage of Otx2+Rlbp:TdTomato+cells of total Rlbp:tdTomato+cells 3d (n=3) and 6dptf (n=6). J) Percentage of TUJ1+EdU+Rlbp:TdTomato+cells of total Rlbp:tdTomato+cells 3d (n=3) and 6dptf (n=8). Scale bars 100 μm, in F 20 μm, in G 50 μm. Significant differences are indicated *: p<0.05, ** p<0.01, ***: p<0.001, Mann-Whitney-test and t-test. Control=control mimic and antagomiR (AR) cocktail, 7+25: let-7 AR and miR-25 mimic cocktail, 7-25-124: let-7 AR, miR-25 and miR-124 mimic cocktail. n: number of cultures of 6-8 mice per culture.

FIG. 13 shows that miR-25/miR-124/let-7 reprogrammed Rlbp1+Müller glia increase in number due to proliferation. A) Schematic of the Rlbp1CreER: tdTomatoflSTOP/flSTOP mouse. B) Experimental design. C-E) Live images of tdTomato+cells after transfection with control. mir-25+let-7AR, or miR-25+let-7AR+miR-124 at 6 dptf. F) Number of Rlbp:TdTomato+cells per field in control and treatment groups (n=6). G/G′-I/I′) Immunofluorescent labeling for tdTomato (Rlbp1) and EdU. J) Number of EdU+Rlbp:tdTomato+cells per field 3-5 days post transfection (dptf, n=5). Scale bars in C-D 200 μm, in G-150 μm. Significant differences are indicated *: p<0.05, ** p<0.01, ***: p<0.001, Mann-Whitney-test. n: number of cultures of 6-8 mice per culture.

FIG. 14 shows that miR-25/miR-124/let-7 can reprogram adult Müller glia. A) Schematic of the Rlbp1CreER: tdTomatoflSTOP/flSTOP mouse. B) Experimental design. C/C″-I/I″) Immunofluorescent labeling for tdTomato (Rlbp1), TUJ1 and DAPI nuclear labeling of MG cultures from one, 2 and 4 month old mice, 6 dptf. J) Graph showing the percentage of TUJ1+RlbpTom+cells/total RlbpTom+cells in cultures from P11 and adult mice (1-4 months pooled). J′) Percentage of TUJ1+Rlbp:TdTomato+cells of total Rlbp:tdTomato+cells from P11 (8 individual experiments), one month old (2 mice pooled, one experiment), 2 months (4 mice pooled, one experiment) and 4 months old mice (8 mice pooled, one experiment). 6 dptf. Scale bars in C-F: 100 μm. G-1: 20 μm. Significant differences are indicated *: p<0.05, p<0.01, ***. p<0.001 Mann-Whitney-test.

FIG. 15 shows that single cell RNAseq of miRNA-mediated reprogramming of MG to RPCs and neurons. A) Cells from all conditions were combined and clustered. tSNE plot shows the relative contributions to each cluster by treatment condition. B) Clusters were designated as a specific cell type by the pattern of gene expression and known marker genes characteristic of that cell type. The largest cell cluster was made up of MG, but other cells, like endothelial cells, microglia and astrocytes, were present as contaminating cell populations in the cultures. C) Cells for the “all” treatment condition (miR-25 and miR-124 mimics, with let-7 AR) are labeled darker gray; the RPC-like cluster was largely made of cells that received this treatment. D) Cells for the control condition are labeled darker gray; these are not present in the “neuron” cluster or the RPC-like cluster. E) Cells expressing Ascl1 are colored in darker gray to show their distribution in the RPC-like cluster. F-I) Violin plots to show the large increases in cell expressing progenitor genes in the “all” (miR-25/124+let-7AR) treatment condition. J) Cells expressing Neurod1 are colored in darker gray (arrow) to show their distribution in the neuron cluster.

FIG. 16 illustrates potential targets of reprogramming miRNAs in MG and RPCs. A) The single cell RNAseq data was analyzed for significant changes across treatment groups using Seurat and the genes that showed declines in the mimic treated conditions, or increased in the let-7AR condition were evaluated for the presence of predicted or known miRNA regulation. Gene-miRNA relationships are plotted as a graph with the nodes as either the genes or miRNAs. Some genes are known targets of miR-124, others are genes in the Rest pathway. and others are those that change the most in the let-7AR or miR-25 conditions. B-C) Violin plots of known targets of miR-124 showing the reduction in the number of cells expressing these genes in the miR-124 treated cells. D-1) Violin plots of known targets of let-7 showing an increase in the number of cells expressing these genes in the let-7 antagomiR treated cells (D-G) and known targets of miR-25 showing a decrease in miR-25 mimic treated cells (H,I). (SEQ ID NO: 36-41) and Klf4 Dkk3 (SEQ ID NO: 42-45), from Targetscan (F, I). Hmg2a, a previously identified target in other cell types, is not altered in expression in the let-7AR treated cells, though a related factor, Hmgn2 is one of the most highly up-regulated genes (D, G).

FIG. 17 illustrates miR-25/124 and let-7 in Müller glia reprogramming—suggested mechanisms. Identification of miRNAs highly expressed in mature Müller glia (MG, left circle: top reprogramming candidate: let-7) and late retinal progenitor cells (RPC, right circle: the top reprogramming candidate: miR-25). Overexpression of RPC-microRNA miR-25 with or without miR-124 (known miRNA to reprogram MG into neurons) and antagonism of MG-miRNA let-7 results in Ascl1 expressing MG-derived progenitors which differentiate into cells expressing neuronal markers (IHC and scRNA-Seq).

### DETAILED DESCRIPTION OF THE INVENTION

The Achaete-scute family bHLH transcription factor 1 (Asci) gene encodes a member of the basic helix-loop-helix (BHLH) family of transcription factors. The protein activates transcription by binding to the E box (5′-CANNTG-3′).

An exemplary nucleic acid sequence encoding human Ascl1 can be found at NCBI Reference Sequence number NG_008950.1 and also provided herein as SEQ ID NO: 1. In another embodiment disclosed herein is a nucleic acid sequence encoding a human Ascl1 amino acid sequence or portion thereof of UniProtKB/Swiss-Prot: P50553.2, provided herein as SEQ ID NO: 2. Ascl1 homologues, e.g., derived from species such as murine, canine, equine, are included herein, without limitation.

The invention described herein is based on the discovery that Ascl1-induced neurogenesis from Müller glia (MG) can be stimulated to produce functional neurons when treated with one or more reprogramming potentiators. In particular, disclosed herein are compositions and methods related to 1) Ascl1 expression in MG and 2) the combined therapy of reprogramming potentiators which cause HDAC inhibition+Jak/STAT inhibition. Disclosed herein we show that Ascl1 expression and the resulting neurogenesis from MG can be significantly enhanced by inhibition of the Jak/STAT pathway, and/or by use of other reprogramming potentiating agents such as mimics or activators of miR-25, and/or miR-124 with inhibitors of the let-7 family. This discovery is surprising, given that STAT significantly increases following injury, and in zebrafish, STAT promotes regeneration. Thus, it would have been expected that inhibition of this pathway would block regeneration of retinal neurons rather than potentiate it. It was also unexpected that a specific cocktail of miRs, miR-25 and miR-124, together with inhibitors of let-7 family members, could turn on endogenous Ascl1, providing another means of potentiating retinal regeneration. These discoveries provide opportunities for regeneration of retinal neurons, including bipolar neurons and amacrine cells, and effective treatment of retinal degenerative disease as well as restoration of vision after injury, disease, or loss.

Regeneration of retinal neurons would be particularly important in a range of degenerative ocular diseases such as, for example and without limitation, retinal degeneration caused by diabetic retinopathy, glaucoma, and age-related macular degeneration. One such retinal degenerative disease is known as central retinal artery occlusion (CRAO), wherein blood flow through the central retinal artery is blocked or occluded often resulting in loss of vision. CRAO is caused by thromboembolus, carotid artery atherosclerosis, giant cell arteritis, aneurysms or arterial spasms. Current treatment paradigms for many of these types of degenerative diseases of the retina, particularly for CRAO, show little to no definitive improvement in outcomes.

### Definitions

All scientific and technical terms used in this application have meanings commonly used in the art unless otherwise specified. As used in this application, the following words or phrases have the meanings specified.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, “retinal neuron” refers to any of the five types of neurons in the retina: photoreceptors, bipolar cells, ganglion cells, horizontal cells, and amacrine cells. In some particular embodiments, the retinal neurons are bipolar neurons and/or amacrine cells.

As used herein, the term “let-7 family of inhibitors” refers to the lethal-7 (Let-7) microRNA (miRNA) family.

As used herein, the terms “nucleic acid sequence” or “polynucleotide” refers to nucleotides of any length which are deoxynucleotides (i.e. DNAs), or derivatives thereof; ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA. DNA-RNA hybrids, oligonucleotides (oligos), or other natural, synthetic, modified, mutated or non-natural forms of DNA or RNA.

MicroRNAs. or “miRNAs”, or “miRs”, are short, non-coding RNAs that regulate gene expression by post-transcriptional regulation of target genes.

“Short hairpin RNAs” or “shRNAs” are synthetic or non-natural RNA molecules. shRNA refers to RNA with a tight hairpin turn used to silence (via RNA interference or RNAi) target gene expression in a cell. An shRNA is typically delivered via an expression vector such as a DNA plasmid or via viral vectors.

The term “vector” refers to, without limitation, a recombinant genetic construct or plasmid or expression construct or expression vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE. The term “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

The term “substantially complementary,” when used to define either amino acid or nucleic acid sequences, means that a particular sequence, for example, an oligonucleotide sequence, is substantially complementary to the sequence of miR-214 referenced. As such, typically the sequences will be highly complementary to the microRNA “target” sequence, and will have no more than 1, 2, 3, 4, 5, 6, 7, 8, 9, or 10 base mismatches throughout the sequence. In many instances, it may be desirable for the sequences to be exact matches. i.e. be completely complementary to the sequence to which the nucleic acid specifically binds, and therefore have zero mismatches along the complementary stretch. As such, highly complementary sequences will typically bind quite specifically to the target sequence region and will therefore be highly efficient in reducing, and/or even inhibiting the biological activity of the target sequence.

Substantially complementary nucleic acid sequences will be greater than about 80 percent complementary (or ‘% exact-match’) to the corresponding target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including 96%, 97%, 98%, 99%, and even 100% exact match complementary to the target to which the designed nucleic acid specifically binds.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include. (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of specifically binding to a target molecule and acting as an antisense construct to alter, reduce, or inhibit the biological activity of the target.

As used herein, “directed against”, in the context of antisense oligonucleotides, means the antisense oligonucleotide binds to a target miRNA and blocks or suppresses activity of the target.

As used herein, the terms “protein”, “peptide”, and “polypeptide” refer to amino acid subunits, amino acid analogs, or peptidomimetics. The subunits may be linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

The term “effective amount” or “therapeutically effective amount” or “prophylactically effective amount”, refer to an amount of an active agent described herein that is effective to provide the desired/intended result and/or biological activity. Thus, for example, in various embodiments, an effective amount of a composition described herein is an amount that is effective to result in regeneration of retinal neurons, and/or to improve or to ameliorate symptoms of and/or to treat retinal degenerative diseases.

When the disclosure herein relates to a small molecule, polypeptide, protein, polynucleotide, nucleic acid, oligonucleotide, antisense, or miRNA, an equivalent or a biologically equivalent of such is intended within the scope of this disclosure. As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least about 70% homology or identity, or at least 80% homology or identity and alternatively, or at least about 85%. or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described below. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%: and wash solutions of about 5×SSC to about 2×SSC. Examples of high stringency conditions include: incubation temperatures of about 55° C. to about 68° C.; buffer concentrations of about I×SSC to about 0.1×SSC; formamide concentrations of about 55% to about 75%; and wash solutions of about I×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “treating” or “treatment” of a retinal degenerative disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the compositions, combination therapy, nucleic acid molecules, and methods disclosed herein for inducing neurogenesis from MG and/or generating functional neurons from MG, beneficial or desired results can include one or more, but are not limited to, alleviation or amelioration of one or more symptoms of retinal degeneration, diminishment of extent of a retinal degenerative condition (including a retinal degenerative disease), stabilized (i.e., not worsening) state of a retinal degenerative condition (including disease), delay or slowing of a retinal degenerative condition (including disease), progression, amelioration or palliation of a retinal degenerative condition (including disease), states of and remission of (whether partial or total) retinal degeneration, whether detectable or undetectable.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, or otherwise in contact with compositions and/or nucleic acid molecules disclosed herein, include without limitation, cells of a human, non-human animal, mammal, or non-human mammal, including without limitation, cells of murine, canine, or non-human primate species. Cells treated, transfected, transformed, or otherwise in contact with compositions and/or nucleic acid molecules disclosed herein are, without limitation, retinal cells, Müller glia, and/or retinal neuronal cells such as retinal neurons and glia. The term “Müller glial” cells “or “Müller glia” or “MG” refer to cells which are found in the vertebrate retina and are support cells for neurons. MG are the most common type of glial cells in the retina. While MG cell bodies are located in the inner nuclear layer of the retina, MG span across the entire retina.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

As used herein, the term “combined therapy” refers to two or more compositions and/or nucleic acid molecules, delivered in combination, for example and without limitation, sequentially, concurrently, simultaneously, and/or step-wise, in order to achieve a therapeutic effect.

As used in the description of the invention and the appended claims, the singular forms “a,” “an” and “the” are intended to include the plural forms as well, unless the context clearly indicates otherwise.

The term “about,” as used herein when referring to a measurable value such as an amount, level or concentration, for example and without limitation, is meant to encompass variations of 20%, 10%, 5%, 1%, 0.5%. or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

In some embodiments, enhancing expression levels of Ascl1, endogenous and/or exogenous, refers to an increase in the amount of expressed Ascl1 as compared to a control sample or explant levels of endogenous and/or exogenous Ascl1, such as, without limitation, untreated, Ascl1 activators alone, nucleic acid molecule encoding Ascl1 alone, and/or Ascl1+HDACi. In some embodiments, Ascl1-induced neurogenesis is increased and/or the production of functional neurons is increased as compared to a control. In some embodiments, Ascl1 expression is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some embodiments, the terms “reprogramming potentiator” or “reprogramming potentiating agent”, used herein interchangeably, refers to a small molecule, polypeptide, protein, polynucleotide, nucleic acid, oligonucleotide, antisense, miRNA, or an equivalent or a biologically equivalent thereof which assists in the process of stimulating neurogenesis from MG in a manner such that functional neurons from the MG are produced. In one embodiment, one or more reprogramming potentiators assist in the process of stimulating neurogenesis and producing functional neurons from MG by inhibiting the HDAC pathway. In another embodiment, one or more reprogramming potentiators assist in the process of stimulating neurogenesis and producing functional neurons from MG by inhibiting the Jak/STAT pathway. In another embodiment, one or more reprogramming potentiators assist in the process of stimulating neurogenesis and producing functional neurons from MG by inhibiting the HDAC+Jak/STAT pathways. In another embodiment, one or more reprogramming potentiators assist in the process of stimulating neurogenesis and producing functional neurons from MG by enhancing and/or increasing endogenous and/or exogenous Ascl1 expression levels.

The terms “acceptable,” “effective,” or “sufficient” when used to describe the selection of any components, ranges, dose forms, etc. disclosed herein intend that said component, range, dose form. etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2, and VP3. Each particle contains approximately 5 VP1 proteins, 5 VP2 proteins and 50 VP3 proteins ordered into an icosahedral structure.

### Compositions

Provided are compositions and/or nucleic acid molecules for retinal regeneration, the potentiation of retinal regeneration, restoration of vision, and for treatment of retinal degenerative disease, damage, or injury.

In one embodiment disclosed herein is a nucleic acid molecule comprising a nucleic acid sequence encoding human Ascl1 in operable linkage with a MG-specific promoter. In another embodiment, a nucleic acid molecule comprising a nucleic acid sequence encoding human Ascl1 in operable linkage with a human Rlbp1 promoter. Rlbp1 (Retinaldehyde binding protein 1) is a robust MG tissue specific promoter capable of driving expression of Ascl1 to MG cells at a level sufficient to induce neurogenesis from MG. In one embodiment, a portion of the Rlbp1 promoter is used for Ascl1 expression in MG. In another embodiment, the portion of the Rlbp1 promoter in operable linkage with the nucleic acid sequence encoding Ascl1 is the Rlbp1 sequence found in SEQ ID NO: 3.

In some embodiments, the nucleic acid molecule comprises a) a nucleic acid sequence encoding Ascl1 and b) a nucleic acid sequence encoding one or more reprogramming potentiators. In one embodiment, the one or more reprogramming potentiators is selected from HDACi, STATi, Jak/STATi and Asci activators. In an additional embodiment, the nucleic acid molecule comprises an MG-specific promoter such as Rlbp1 or a portion thereof.

In one embodiment, the nucleic acid molecule comprises a nucleic acid sequence encoding Ascl1 and a nucleic acid sequence encoding one or more HDACi peptides or proteins. In another embodiment, the nucleic acid molecule comprises a nucleic acid sequence encoding Ascl1 and a nucleic acid sequence encoding one or more STATi peptides or proteins. In another embodiment, the nucleic acid molecule comprises a nucleic acid sequence encoding Ascl1 and a nucleic acid sequence encoding one or more Jak/STATi peptides or proteins. In another embodiment, the nucleic acid molecule comprises a nucleic acid sequence encoding Ascl1 and a nucleic acid sequence encoding one or more HDACi, STATi, and/or Jak/STATi peptides or proteins. In another embodiment, the nucleic acid molecule comprises a) a nucleic acid sequence encoding Ascl1 and a nucleic acid sequence encoding one or more Ascl1 activator peptides, proteins or RNAi molecules such as miRs and shRNAs.

In one embodiment disclosed herein is a nucleic acid molecule comprises a) a nucleic acid sequence encoding Ascl1 and b) a nucleic acid sequence encoding one or more HDACi peptides selected from the group consisting of 16cyc-HxA. 16lin-HxA. and 16KA. In another embodiment, a nucleic acid molecule comprises a) a nucleic acid sequence encoding Ascl1 and b) a nucleic acid sequence encoding one or more Jak/STATi proteins/peptides selected from the group consisting of Socs1, Socs2, Socs3, Socs4, Socs5, Socs6, Socs7, CIS. and XpYL. In another embodiment, a nucleic acid molecule comprises a) a nucleic acid sequence encoding Asci, b) a nucleic acid sequence encoding one or more HDACi peptides selected from the group consisting of 16cyc-HxA: Ac-RRR-cyclic(isoDVADap)REIRRYQHxA (SEQ ID NO: 4), 161lin-HxA: Ac-RRR-TVALREIRRYQHxA (SEQ ID NO: 5), and 16KA: Ac-RRR-cyclic(isoDVADap)REIRRYQKA (SEQ ID NO: 6), and c) a nucleic acid sequence encoding one or more Jak/STATi proteins/peptides selected from the group consisting of Socs1, Socs2, Socs3, Socs4, Socs5, Socs6, Socs7, CIS, and XpYL. Exemplary HDACi peptides are known in the art, see also Wang et al., AACR Journals, 6 Mar. 2019; DOI: 10.1158/0008-5472.CAN-18-1421. Exemplary Socs nucleic acid and amino acid sequences are listed, without limitation, in Table 1.

TABLE 1

| Jak/STATi | | |
|---|---|---|
| Reprogramming | | Nucleic Acid |
| Repotentiator Proteins | Protein Sequence | Sequence |
| Human SOCS1 | SEQ ID NO: 7 | SEQ ID NO: 8 |
| (Suppressor of Cytokine | (NCBI Reference: | (NCBI Reference |
| Signaling 1) | NP_003736.1) | NM_003745.1) |
| Human SOCS2 | SEQ ID NO: 9 | SEQ ID NO: 10 |
| (Suppressor of Cytokine | (NCBI Reference: | (NCBI Reference: |
| Signaling 2) | NP_001257400.1) | NM_003877.4) |
| Human SOCS3 | SEQ ID NO: 11 | SEQ ID NO: 12 |
| (Suppressor of Cytokine | (NCBI Reference: | (NCBI Reference: |
| Signaling 3) | NP_003946.3) | NM_003955.4) |
| Human SOCS4 | SEQ ID NO: 13 | SEQ ID NO: 14 |
| (Suppressor of Cytokine | (NCBI Reference: | (NCBI Reference: |
| Signaling 4) | NP_955453.1) | NM_199421.2) |
| Human SOCS5 | SEQ ID NO: 15 | SEQ ID NO: 16 |
| (Suppressor of Cytokine | (NCBI Reference: | (NCBI Reference: |
| Signaling 5) | NP_054730.1) | NM_014011.4) |
| Human SOCS6 | SEQ ID NO: 17 | SEQ ID NO: 18 |
| (Suppressor of Cytokine | (NCBI Reference: | (NCBI Reference: |
| Signaling 6) | NP_004223.2) | NM_004232.4) |
| Human SOCS7 | SEQ ID NO: 19 | SEQ ID NO: 20 |
| (Suppressor of Cytokine | (NCBI Reference: | (NCBI Reference: |
| Signaling 7) | NP_0055413.2) | NM_014598.4) |

In one embodiment disclosed herein is a nucleic acid molecule comprises a) a nucleic acid sequence encoding Ascl1 and b) a nucleic acid sequence encoding 16cyc-HxA, and c) a nucleic acid sequence encoding Socs1-7, CIS and/or XpYL. In one embodiment disclosed herein is a nucleic acid molecule comprises a) a nucleic acid sequence encoding Asci and b) a nucleic acid sequence encoding 16lin-HxA, and c) a nucleic acid sequence encoding Socs1-7, CIS and/or XpYL. In one embodiment disclosed herein is a nucleic acid molecule comprises a) a nucleic acid sequence encoding Ascl1 and b) a nucleic acid sequence encoding 16KA, and c) a nucleic acid sequence encoding Socs1-7, CIS and/or XpYL. In another embodiment. In one embodiment disclosed herein is a nucleic acid molecule comprises a) a nucleic acid sequence encoding Ascl1 i and b) a nucleic acid sequence encoding 16cyc-HxA, 16lin-HxA, and/or 16KA.

In another embodiment disclosed herein is a nucleic acid molecule comprises a) a nucleic acid sequence encoding Ascl1 i and b) nucleic acid sequence encoding Socs1. In another embodiment disclosed herein is a nucleic acid molecule comprises a) a nucleic acid sequence encoding Ascl1 and b) nucleic acid sequence encoding Socs2. In another embodiment disclosed herein is a nucleic acid molecule comprises a) a nucleic acid sequence encoding Ascl1 and b) nucleic acid sequence encoding Socs3. In another embodiment disclosed herein is a nucleic acid molecule comprises a) a nucleic acid sequence encoding Ascl1 and b) nucleic acid sequence encoding Socs4. In another embodiment disclosed herein is a nucleic acid molecule comprises a) a nucleic acid sequence encoding Ascl1 and b) nucleic acid sequence encoding Socs5. In another embodiment disclosed herein is a nucleic acid molecule comprises a) a nucleic acid sequence encoding Ascl1 and b) nucleic acid sequence encoding Socs6. In another embodiment disclosed herein is a nucleic acid molecule comprises a) a nucleic acid sequence encoding Ascl1 and b) nucleic acid sequence encoding Socs7. In another embodiment disclosed herein is a nucleic acid molecule comprises a) a nucleic acid sequence encoding Ascl1 and b) nucleic acid sequence encoding CIP. In another embodiment disclosed herein is a nucleic acid molecule comprises a) a nucleic acid sequence encoding Ascl1 and b) nucleic acid sequence encoding XpYL.

In an alternative embodiment disclosed herein is a nucleic acid molecule comprising a) a nucleic acid sequence encoding Ascl1 and b) a nucleic acid sequence generating one or more Ascl1 activators, wherein the Ascl1 activators are shRNAs, wherein the shRNAs are selected from the group consisting of miR-25, miR-124, and let-7 family inhibitors. In further embodiment, the nucleic acid molecule further comprises a nucleic acid sequence encoding one or more reprogramming potentiators.

In alternative embodiments, the composition comprises (a) one or more Ascl1 gene expression activators; (b) one or more HDAC inhibitors; and (c) one or more Jak/STAT inhibitors. In another embodiment, the composition comprises one or more reprogramming potentiating agents. The reprogramming potentiating agents, in some embodiments, are selected from (i) one or more STAT signaling pathway inhibitors; (ii) miR-25; and (iii) one or more let-7 family inhibitors. In one embodiment of the composition, the agents of (c) comprise one or more STAT signaling pathway inhibitors or one or Jak/STAT signaling pathway inhibitors. In another embodiment, the agents of (c) comprise miR-25; and one or more let-7 family inhibitors. In some embodiments, the agents of (c) comprise one or more STAT signaling pathway inhibitors: miR-25; and one or more let-7 family inhibitors.

In one embodiment, the HDAC signaling pathway inhibitor (HDACi) is selected from the group consisting of peptidomimetics, small molecule inhibitors, oligonucleotides, peptides and proteins. Representative examples of small molecule HDACi include, but are not limited to, trichostatin A (TSA), Istodax™ also known as (Pro)/romidepsin, Beleodaq™, also known as (Pro)/belinostat. Farydak™, also known as (Pro)/panobinostat, and Zolinza™, also known as (Pro)/vorinostat, Quisinostat, Abexinostat, Givinostat, Resminostat, Phenylbutyrate, Valproic Acid, Depsipeptide, Entinostat, Mocetinostat, and Tubastatin A. Exemplary HDACi peptides are, without limitation, 16cyc-HxA, 16lin-HxA and 16KA.

In one embodiment, the STAT signaling pathway inhibitor or Jak/STATi is selected from the group consisting of natural compounds, peptidomimetics, peptides, proteins, small molecules and oligonucleotides. Examples of endogenous STAT pathway inhibitors, include, but are not limited to, suppressor of cytokine signaling (SOCS) proteins, phosphatases, and protein inhibitor of activated STAT (PIAS) proteins. Such endogenous inhibitors provide a basis for therapeutic molecules and compounds for STAT inhibition. In one embodiment, protein or peptide inhibitors of STAT include, for example and without limitation, Socs1, Socs2, Socs3, Socs4, Socs5, Socs6, Socs7, CIS, and/or XpYL. In one embodiment, a peptidomimetic inhibitor of STAT is ISS610. In another embodiment, small molecule inhibitors of STAT and/or Jak/STAT include, without limitation, STA-21, LLL3, S31-201, Stattic, OPB-31121, OPB-51602, SH-4-54, Tofactinib, Ruxolitinib, Baricitinib, Oclacitinib, AZD1480, and Dasatinib. In one embodiment, the STAT signaling pathway inhibitor is an inhibitor of STAT3. An exemplary STAT3 inhibitor includes, but is not limited to, SH-4-54. See also Fagard et al. JAKSTAT. 2013 Jan. 1; 2(1): e22882. In another embodiment, STAT and/or Jak/STAT inhibitors include natural compounds such as Butein and Capsaicin.

In some embodiments, the inhibitor, mimic, activator, or antagomir is an oligonucleotide or a nucleotide sequence. The invention thus provides nucleotide constructs for use in the compositions or nucleic acid molecules and methods described herein.

In one representative example, the STAT inhibitor is a nucleotide sequence encoding a Socs1-7 amino acid sequences selected from the group consisting of SEQ ID NOS: 7-20. In one embodiment, microRNA mmu-miR-25-3p has the mature sequence: CAUUGCACUUGUCUCGGUCUGA (SEQ ID NO: 21; Mature Accession: MIMAT0000652). In one embodiment, the sequence of the miR-25 mimic is CAUUGCACUUGUCUCGGUCUGA (SEQ ID NO: 22). In one embodiment, the microRNA mmu-miR-124-3p has the mature sequence: UAAGGCACGCGGUGAAUGCC (SEQ ID NO: 23; Mature Accession: MIMAT0000134). In one embodiment, the sequence for miR-124 mimic is UAAGGCACGCGGUGAAUGCC (SEQ ID NO: 24). Representative examples of the let-7 family inhibitor include, but are not limited to, mmu-let-7a-5p and mmu-let-7c-5p with the mature sequences UGAGGUAGUAGGUUGUAUAGUU (SEQ ID NO: 25; Mature Accession: MIMAT0000521) and UGAGGUAGUAGGUUGUAUGGUU (SEQ ID NO: 26; Mature Accession: MIMAT0000523) respectively. Representative examples of the let-7 family antagomiRs include, but are not limited to, let-7a antagomiR (UGAGGUAGUAGGUUGUAUAGUU; SEQ ID NO: 27) and let-7c antagomiR (UGAGGUAGUAGGUUGUAUGGUU; SEQ ID NO: 28).

Such oligonucleotide/polynucleotide/nucleotide constructs may be delivered by viral or non-viral means. One example of viral delivery is adeno-associated virus (AAV). Other examples include retrovirus, lentivirus, and baculovirus delivery. One example of a non-viral method of miR delivery is cell penetrating peptide (CPP). Nucleotide constructs may also be modified, such as through chemical modification, to improve their stability and/or suitability for delivery. In some embodiments, a nucleic acid sequence is modified by locked nucleic acids and/or phosphorothioate linkages. In some embodiments, a delivery system is selected for improved bioavailability, such as PEGylated liposomes, lipidoids, or biodegradable polymers, as examples.

### Nucleic Acid Molecules and Combined Therapy Compositions

Additionally provided herein are nucleic acid molecules encoding Ascl1 for use in enhancement or stimulation of retinal regeneration, potentiation of retinal regeneration, restoration of vision, and for treatment of retinal degenerative disease, damage, or injury. In some embodiments, the nucleic acid molecule encoding Ascl1 is administered concurrently or sequentially with the combined therapy of a composition which comprises one or more HDACi: and a composition which comprises one or more reprogramming potentiating agents. In one embodiment, the reprogramming potentiating agent is a STATi. The combined therapy of HDACi and STATi results in the synergistic effect of generating functional neurons as a result of Ascl1-induced neurogenesis from MG compared to Ascl1 expression alone and/or Ascl1 expression+HDACi.

The reprogramming potentiating agents, in some embodiments, are selected from (i) one or more STAT signaling pathway inhibitors; (ii) miR-25 and/or miR-124; and (iii) one or more let-7 family inhibitors. In one embodiment, the combined therapy is a composition comprising one or more HDACi and a composition comprising one or more STAT signaling pathway inhibitors. Representative examples of the HDACi include, but are not limited to, trichostatin A (TSA), Istodax™ also known as (Pro)/romidepsin, Beleodaq™, also known as (Pro)/belinostat, Farydak™, also known as (Pro)/panobinostat, and Zolinza™, also known as (Pro)/vorinostat.

In one embodiment, the STAT signaling pathway inhibitor is selected from the group consisting of peptidomimetics, small molecule inhibitors and oligonucleotides. Examples of endogenous STAT pathway inhibitors, include, but are not limited to, suppressor of cytokine signaling (SOCS) proteins, phosphatases, and protein inhibitor of activated STAT (PIAS) proteins. Such endogenous inhibitors provide a basis for therapeutic molecules and compounds for STAT inhibition. In one embodiment, the STAT signaling pathway inhibitor is an inhibitor of STAT3. An exemplary small molecule STAT3 inhibitor is, but is not limited to, SH-4-54. See also Fagard et al. JAKSTAT. 2013 Jan. 1; 2(1): e22882.

In one embodiment, the nucleic acid molecule for use in inducing or stimulating retinal neurogenesis from MG comprises a nucleic acid sequence encoding Ascl1. In another embodiment, the nucleic acid sequence encoding Ascl1 comprises a native Ascl1 promoter sequence. In another embodiment, the nucleic acid sequence encoding Asci comprises a non-native Ascl1 promoter. In another embodiment, a non-native Asci promoter is a MG-specific promoter such as, for example and without limitation, a retinaldehyde binding protein 1 (Rlbp1) promoter or portion thereof, glial fibrillary acidic protein (GFAP) promoter, vimentin (VIM) promoter, Hes1 promoter, or CD44 promoter. In another embodiment, a non-native Ascl1 promoter is a ubiquitous promoter such as, for example and without limitation, a CMV promoter, CAG promoter or miniCMV promoter.

In some embodiments, the vector disclosed herein is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector. a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to a polynucleotide. In some embodiments, the vector further comprises one or more selectable markers.

In some embodiments, the vector disclosed herein is an AAV vector with low toxicity. In some embodiments, the AAV vector does not incorporate into the host genome, thereby having a low probability of causing insertional mutagenesis. In some embodiments, the AAV vector can encode a range total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof.

In some embodiments, the vector disclosed herein is a lentiviral vector. In one embodiment, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art. In some embodiments, exemplary lentiviral vectors that may be used in relation to any of the herein described compositions, nucleic acid molecules and/or methods, and can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, a equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/NMV) vector, a modified Visna/maedi virus (VNV/NMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

In some embodiments, expression vector or viral vector disclosed herein is used to transfect, transform, or come in contact with a cell which is a eukaryotic cell. In some embodiments, the cell is an animal cell. In some embodiments, the cells is a zebrafish cell. In some embodiments, the cell is a mammalian cell. In some embodiments, the cell is a bovine, murine, feline, equine, porcine, canine, simian, or human cell. In particular embodiments, the cell is a retinal neuron or MG or amacrine cell of an animal or mammal.

In some embodiments of the compositions and methods of the disclosure, a cell of the disclosure is a retinal cell, such as a Müller glial (MG) cell, or a rod or cone photoreceptor cell. In some embodiments, the cell is a neuronal cell. In some embodiments, a neuronal cell of the disclosure is a neuron of the retina. In some embodiments, a neuron cell of the disclosure is a neuron of an optic nerve. In some embodiments, a neuron cell of the disclosure is a neuroglial or a glial cell. In some embodiments, a cell is a bipolar neuron or an amacrine cell. In some embodiments, a cell of the disclosure is an astrocyte. In some embodiments, cells of the disclosure are macroglia or microglia.

In some embodiments of the compositions and methods of the disclosure, a cell of the disclosure is a cultured cell.

In some embodiments of the disclosure, a cell is in vivo, in vitro, ex vivo, or in situ.

In some embodiments, a cell of the disclosure is autologous or allogeneic and used for transplantation.

In some embodiments, a cell of the disclosure is a stem cell-derived or an embryonic stem cell-derived retinal cell. In some embodiments, the cell is derived from an iPS cell-derived retinal cell.

In some embodiments, a cell is a packaging cell or a producer cell for production of a viral particle.

In some embodiments, provided herein are viral particles comprising, consisting of, or consisting essentially of a vector comprising, consisting of, or consisting essentially of a polynucleotide sequence encoding an Ascl1 protein.

In general, methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector. adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

The resulting packaged expression systems may then be introduced via an appropriate route of administration, discussed in detail with respect to the method aspects disclosed herein.

Also provided herein is a composition comprising any one or more of the combined therapy of Ascl1 activators and/or HDACi+STATi, and/or a nucleic acid sequence encoding the Ascl1 protein or a vector comprising the nucleic acid sequence, and/or miRs disclosed herein, and a carrier. In some embodiments, the carrier is a pharmaceutically acceptable carrier.

Pharmaceutical compositions disclosed herein include one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine: antioxidants; chelating agents such as EDTA or glutathione: adjuvants (e.g., aluminum hydroxide): and preservatives. Compositions of the disclosure may be formulated for intraocular administration.

### METHODS OF THE INVENTION

Described herein are methods for inducing retinal regeneration in a subject. Also provided are methods for enhancing retinal regeneration, improving Ascl1-induced retinal neurogenesis, potentiating retinal regeneration, restoring vision, and treating retinal degenerative disease, damage, or injury.

In one embodiment is a method for inducing retinal regeneration in a subject comprising: a) administering to a retina of the subject the nucleic acid molecules disclosed herein. In particular, in one embodiment, a method for inducing retinal regeneration in a subject comprises: a) administering to a retina of the subject a nucleic acid molecule comprising a nucleic acid sequence encoding Ascl1 and a nucleic acid sequence encoding one or more reprogramming potentiators, wherein Ascl1 induces neurogenesis from MG. and wherein the one or more reprogramming potentiators stimulate production of functional neurons from Ascl1-induced MG. The combination of expressed Ascl1 and the one or more reprogramming potentiator proteins/peptides results in the synergistic effect of generating functional neurons as a result of Ascl1-induced neurogenesis from MG compared to Ascl1 expression alone. In another embodiment, a method for inducing retinal regeneration in a subject comprises administration of a nucleic acid molecule comprising a nucleic acid sequence encoding Ascl1 in operable linkage with an MG-specific promoter. In another embodiment, a method for inducing retinal regeneration in a subject comprises administration of a nucleic acid molecule comprising a nucleic acid sequence encoding Ascl1 in operable linkage with a Rlbp1 promoter.

In some embodiments, the method comprises (a) administering to the subject a composition comprising a nucleic acid molecule encoding Ascl1 Achaete-scute-like 1, or one or more Ascl11 gene expression activators; (b) administering to the subject a composition comprising one or more histone deacetylase inhibitors (HDACi); and (c) administering to the subject a composition comprising a reprogramming potentiating agent. In some embodiments, the reprogramming potentiating agent is: (i) one or more Signal Transducer and Activator of Transcription (STAT) signaling pathway inhibitors; and/or (ii) microRNA 25 (miR-25); and one or more let-7 family inhibitors. In some embodiments, the composition for retinal regeneration comprises (a) one or more miR-25 activators or mimics thereof: (b) one or more microRNA 124 (miR-124) activators or mimics thereof; and (c) one or more let-7 family inhibitors or antagomirs. In some embodiments, the one or more Ascl1 gene expression activators, the one or more HDACi; the one or more STAT signaling pathway inhibitors; the miR-25: and the one or more let-7 family inhibitors are administered sequentially. In some embodiments, the one or more Ascl1 gene expression activators, the one or more HDACi; the one or more STAT signaling pathway inhibitors; the miR-25; and the one or more let-7 family inhibitors are administered concurrently.

In one embodiment, the composition administered in step (c) comprises one or more STAT signaling pathway inhibitors. In another embodiment, the compositions administered in step (c) comprise microRNA miR-25: and one or more let-7 family inhibitors. In a further embodiment, the compositions administered in step (c) comprise each of one or more STAT signaling pathway inhibitors; miR-25; and one or more let-7 family inhibitors.

Also provided herein are methods for inducing retinal regeneration comprising administering to a subject a composition as described herein. In some embodiments, the methods are effective to increase the number of Müller glial-derived neurons, to induce Müller glial cells to enter the mitotic cell cycle, and/or to generate new retinal neurons, including the generation of new bipolar neurons and/or amacrine cells. In some embodiments of the method, the number of retinal neurons increases by at least 25% relative to a baseline level or other reference amount representative of an untreated retina. In other embodiments, the number of retinal neurons increases by at least 40%. In some embodiments, the number of retinal neurons increases by 10%. 20%, 50%, 100%, 150%, 200%, or more.

The subject is typically a mammal, such as a human or veterinary subject. In one embodiment, the subject is an adult. The subject, in some embodiments, has a retinal degenerative disease. Examples of such retinal degenerative diseases include, but are not limited to, Age-related Macular Degeneration (AMD), Retinitis Pigmentosa (RP), Diabetic Retinopathy (DR), Central Retinal Artery Occlusion (CRAO), Vitreoretinopathy, and Glaucoma.[

Administration and Dosage

The compositions are administered in any suitable manner, often with pharmaceutically acceptable carriers. Suitable methods of administering compositions, compounds, molecules, nucleic acids, and vectors in the context of the present invention to a subject's eye or retina are available, and, although more than one route can be used to administer a particular composition, a particular route can often provide a more immediate and more effective reaction than another route. For treatment of the retina, intraocular injection, such as, for example and without limitation, intravitreal injection and subretinal injection are the most common routes of delivery to the retina. In some embodiments, however, periocular, suprachoroidal, systemic, or topical administration is more suitable for efficacy and safety of delivery.

The dose administered to a patient, in the context of the disclosure herein, should be sufficient to result in a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease or injury. An amount adequate to accomplish this is defined as a “therapeutically effective dose.”

Routes, order and/or frequency of administration of the therapeutic compositions disclosed herein, as well as dosage, will vary from individual to individual, and may be readily established using standard techniques. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLE EMBODIMENTS

Embodiment 1) A composition for retinal regeneration comprising: (a) one or more Ascl1 gene expression activators; (b) one or more HDACi inhibitors; and (c) one or more reprogramming potentiating agents selected from: (i) one or more STAT signaling pathway inhibitors; (ii) miR-25, or an activator or mimic thereof; and (iii) one or more let-7 family inhibitors.

Embodiment 2) The composition of a preceding embodiment, wherein the agents of (c) comprise one or more STAT signaling pathway inhibitors.

Embodiment 3) The composition of any of a preceding embodiment, wherein the agents of (c) comprise miR-25; and one or more let-7 family inhibitors.

Embodiment 3A) The composition of embodiment 3, wherein the one or more activators of Ascl1 gene expression are the agents of (c).

Embodiment 4) The composition of any of a preceding embodiment, wherein the agents of (c) comprise one or more STAT signaling pathway inhibitors; miR-25, or an activator or mimic thereof; and one or more let-7 family inhibitors.

Embodiment 5) The composition of any of a preceding embodiment, wherein the HDACi is trichostatin A (TSA), romidepsin, belinostat, panobinostat, or vorinostat.

Embodiment 6) The composition of any of a preceding embodiment, wherein the STAT signaling pathway inhibitor is an inhibitor of STAT3.

Embodiment 7) A composition for retinal regeneration comprising: (a) miR-25, or an activator or mimic thereof; (b) miR-124, or an activator or mimic thereof; and (c) one or more let-7 family inhibitors.

Embodiment 8) A method for inducing retinal regeneration comprising administering to a subject the composition of any of any of a preceding embodiment.

Embodiment 9) A method for increasing the number of Müller glial-derived neurons in the retina of a subject, the method comprising administering to a subject the composition of any of a preceding embodiment.

Embodiment 10) A method of treating retinal disease, damage, or degeneration comprising administering to a subject the composition of any of a preceding embodiment.

Embodiment 11) The method of any of a preceding embodiment, wherein the one or more Ascl1 gene expression activators, the one or more HDACi; the one or more STAT signaling pathway inhibitors; the miR-25: and the one or more let-7 family inhibitors are administered sequentially.

Embodiment 12) The method of any of a preceding embodiment, wherein the one or more Ascl1 gene expression activators, the one or more HDACi inhibitors; the one or more STAT signaling pathway inhibitors; the miR-25; and the one or more let-7 family inhibitors are administered concurrently.

Embodiment 13) The method of any of a preceding embodiment, wherein the number of retinal neurons increases by at least 40%.

Embodiment 14) The method of any of a preceding embodiment, wherein the subject is an adult.

Embodiment 15) A method for inducing retinal regeneration in a subject comprising. a) administering to a retina of the subject a polynucleotide sequence encoding Ascl1 (Achaete-scute-like family bHLH transcription factor 1), wherein expression of Ascl1 induces neurogenesis from Müller glia (MG): and b) sequentially or concurrently administering to the retina of the subject a combined therapy comprising a composition comprising one or more TSA/HDAC inhibitors (HDACi); and a composition comprising one or more STAT signaling pathway inhibitors (STATi), wherein the combined therapy stimulates production of functional neurons from the Ascl1-induced neurogenesis from the MG.

Embodiment 16) The method of any of a preceding embodiment, wherein a vector comprises the polynucleotide sequence encoding Ascl1.

Embodiment 17) The method of any of a preceding embodiment, wherein the vector is a non-viral vector or a viral vector.

Embodiment 18) The method of any of a preceding embodiment, wherein the viral vector is an adeno-associated viral (AAV) vector or a lentiviral vector.

Embodiment 19) The method of any of a preceding embodiment, the polynucleotide sequence comprises a promoter.

Embodiment 20) The method of any of a preceding embodiment, wherein the promoter is a retinal or MG-specific promoter.

Embodiment 21) The method of claim of any of a preceding embodiment, wherein administering to the retina is intravitreal or subretinal injection.

Embodiment 22) In a method of Ascl1-induced neurogenesis from MG, the improvement comprising co-administration of one or more HDACi and one or more STATi.

Embodiment 23) The method of any of a preceding embodiment, wherein co-administration is concurrent or sequential.

Embodiment 24) The methods of any of a preceding embodiment, further comprising the administration of one or more Ascl1 gene expression activators, one or more reprogramming potentiating agents, or a combination thereof.

Embodiment 25) The method of any of a preceding embodiment, wherein the one or more Ascl1 gene expression activators are selected from the group consisting of 1) miR-25, or an activator or mimic thereof; 2) miR-124, or an activator or mimic thereof; 3) one or more let-7 family inhibitors or antagomirs; and 4) a combination thereof.

Embodiment 26) The method of any of a preceding embodiment, wherein the one or more reprogramming potentiating agents are selected from the group consisting of a combination thereof.

Embodiment 27) The method of any of a preceding embodiment, wherein miR-25, miR-124. or let7 is expressed by a short hairpin RNA (shRNA).

Embodiment 28) The method of any of a preceding embodiment, wherein a vector comprises the shRNA.

Embodiment 29) A nucleic acid molecule comprising a nucleic acid sequence encoding Ascl1 and a shRNA.

Embodiment 30) The nucleic acid molecule of any of a preceding embodiment, wherein the shRNA is a miR-25, miR-124, or let-7 shRNA.

Embodiment 31) A nucleic acid molecule comprising a nucleic acid sequence encoding Ascl1 and a MG-specific promoter sequence.

Embodiment 32) A nucleic acid molecule comprising a nucleic acid sequence encoding Ascl1 and a Rlbp1 promoter sequence.

### Examples

The following examples are presented to illustrate the present invention and to assist one of ordinary skill in making and using the same. The examples are not intended in any way to otherwise limit the scope of the invention.

### Example 1: STAT Pathway Activation Limits the Ascl1-Mediated Chromatin Remodeling Required for Neural Regeneration from Müller Alia in Adult Mouse Retina

This Example demonstrates that STAT pathway activation reduces the efficiency of Ascl1-mediated reprogramming in Müller glia, potentially by directing Ascl1 to inappropriate targets. Müller glia can serve as a source for retinal regeneration in some non-mammalian vertebrates. Recently we found that this process can be induced in mouse Müller glia after injury, by combining transgenic expression of the proneural transcription factor Ascl1 and the HDAC inhibitor TSA. However, new neurons are only generated from a subset of Müller glia in this model, and identifying factors that limit Ascl1-mediated MG reprogramming could potentially make this process more efficient, and potentially useful clinically. One factor that limits neurogenesis in some non-mammalian vertebrates is the STAT pathway activation that occurs in Müller glia in response to injury. In this report, we tested whether injury induced STAT activation hampers the ability of Ascl1 to reprogram Müller glia into retinal neurons. Using a STAT inhibitor, in combination with our previously described reprogramming paradigm, we found a large increase in the ability of Müller glia to generate neurons, similar to those we described previously. Single-cell RNA-seq showed that the progenitor-like cells derived from Ascl1-expressing Müller glia have a higher level of STAT signaling than those that become neurons. Using Ascl1 ChIP-seq and DNase-seq, we found that developmentally inappropriate Ascl1 binding sites (that were unique to the overexpression context) had enrichment for the STAT binding motif.

### INTRODUCTION

Functional regeneration of retinal neurons occurs naturally in teleost fish and many amphibians¹⁻⁶. In zebrafish, the Müller glia (MG) respond to a variety of injury models by generating cells that resemble multipotent progenitor cells found in the developing retina. These MG-derived progenitors have the capacity to produce all types of retinal neurons and restore visual function⁷. In amphibians and embryonic birds, the pigmented epithelial cells undergo a similar transition to retinal progenitor cells, and can regenerate a new, laminated retina^{8,9}.

In adult birds and mammals, functional regeneration does not occur spontaneously after retinal injury. Neurotoxic damage to retinal neurons in newly hatched chicks causes the MG to undergo the initial stages of the process that occurs in fish, but few of the MG-derived progenitors go on to make neurons and it is not known whether the few regenerated MG-derived neurons can functionally integrate into the existing retinal circuitry¹. Injury to the mammalian retina has been studied most extensively in rodents, and as in the bird, retinal injury does not initiate a spontaneous regenerative response¹⁰. Attempts to stimulate MG proliferation after injury by stimulating specific signaling pathways with growth factors and small molecules have led to some evidence for new neurogenesis¹¹; however, none of these treatments have been sufficient to regenerate functional neurons from MG in mice^{12,14}.

Recently, we have found that transgenic overexpression of the proneural bHLH Ascl1 enables MG to generate functional neurons in mice. We found that in mice up to two weeks old, Ascl1 alone can induce neurogenesis from MG after N-Methyl-D-aspartic acid (NMDA) excitotoxic damage¹⁵. More recently, we demonstrated that Ascl1 and the HDAC inhibitor trichostatin A (TSA) were together sufficient to induce MG to regenerate functional neurons after retinal injury in adult mice¹⁸. While these studies were encouraging and demonstrated for the first time that new neurons generated in adult mice can be integrated into the mature retinal circuit, the majority of the MG in treated retinas did not undergo neurogenesis.

In the course of our analysis of single-cell RNA-seq data from the previous study, we noticed that those MG that failed to reprogram to neurons had a high level of STAT3 signaling. MG from fish, birds and mammals all respond to retinal damage by rapidly activating STAT3 signaling¹⁷⁻²⁰. In the mouse retina, STAT3 signaling in MG is associated with reactive “gliosis”; however, in the fish retina, STAT3 is required for damage-induced MG-mediated neuronal regeneration, and JAK/STAT activation is sufficient to induce retinal regeneration in the absence of injury^{17,21,22}. In the chick retina, however, experiments have suggested that STAT signaling instead may limit regeneration: inhibition of STAT3 increases the neurogenesis from MG in damaged retinas²⁰. Since an increase in STAT signaling is well known to occur in glia after neuronal injury, we tested whether the injury response was potentially reducing Ascl1-mediated reprogramming. We tested this hypothesis by inhibition of STAT signaling, and found that a STAT inhibitor, in combination with our previously described treatment paradigm, doubled the efficiency of neuron regeneration in vivo. Analysis of ChIP-seq data of Ascl1-expressing MG suggests that STAT signaling may act to redirect Ascl1 to inappropriate regulatory sites in the genome, and implicate the Id1 and Id3 inhibitors of bHLH factors in this process. Our results extend previous work in fish and birds on the importance of JAK/STAT signaling in retinal regeneration. Together with the results from the non-mammalian vertebrates, it appears that an initial activation of the STAT pathway may be necessary to start the regeneration process, but sustained activation of the pathway may then become limiting.

Results

STAT Pathway Inhibition Improves MG-Derived Neurogenesis In Vivo

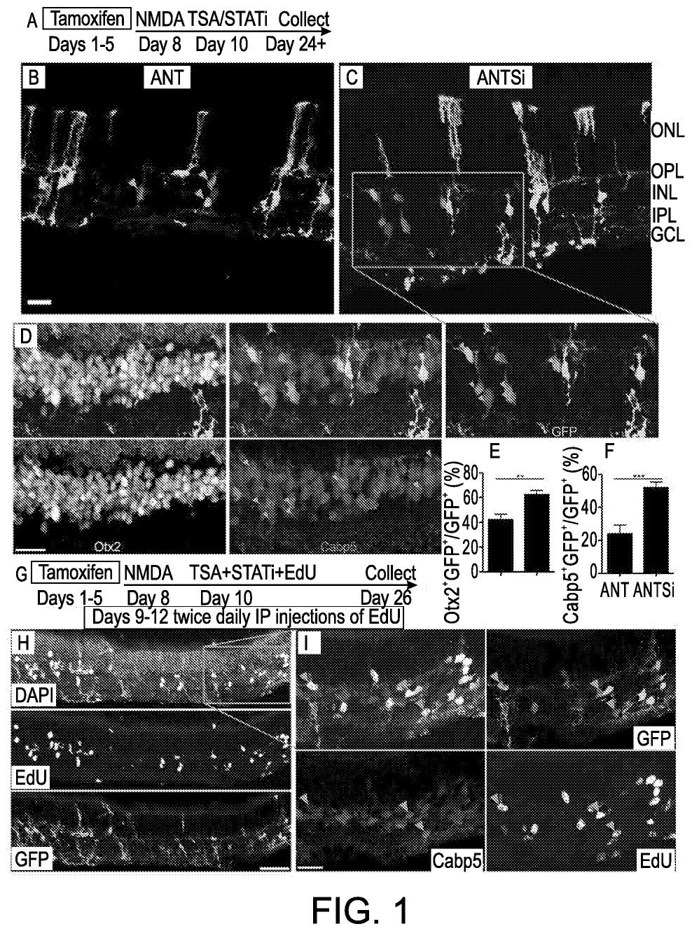

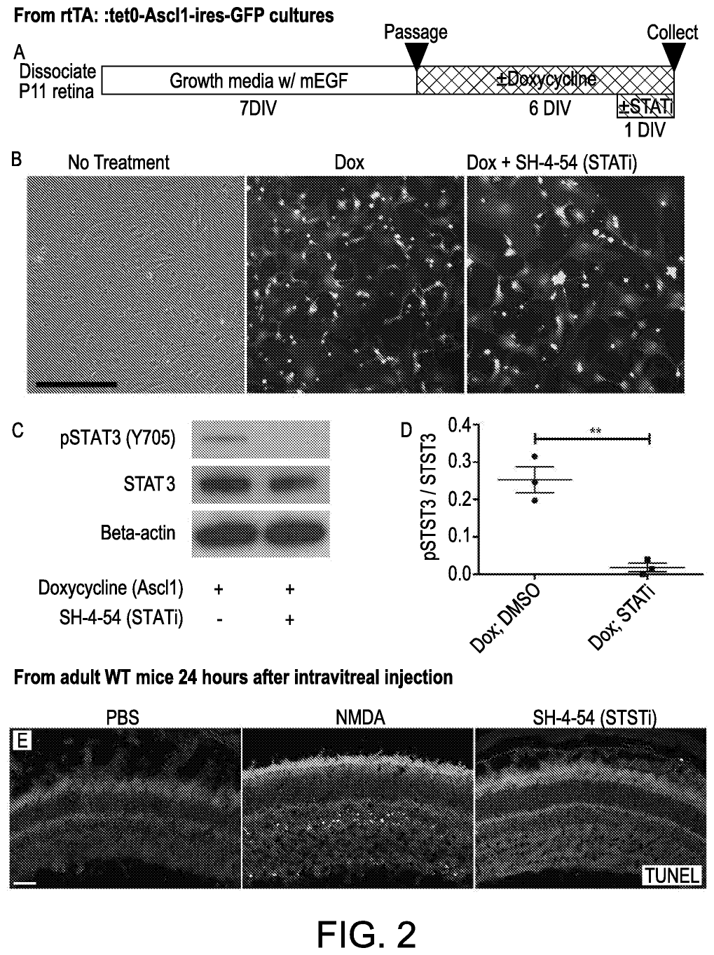

To test whether the activation of the STAT pathway in MG reduces their ability to be effectively reprogrammed by Ascl1, we inhibited this pathway in an experimental system in which Ascl1 can be targeted specifically to MG. We previously described^{15,16} a MG-specific tamoxifen inducible mouse model of Ascl1-overexpression (Glast-CreER:Rosa-flox-stop-LNL-tTA:tetO-mAscl1-ires-GFP). Adult mice received up to five consecutive daily intraperitoneal (IP) injections of tamoxifen (1) to induce Ascl1 expression in the MG, followed by (2) an intravitreal injection of NMDA to induce degeneration of inner retinal neurons, and then (3) an intravitreal injection of either TSA or TSA and the potent STAT inhibitor, SH-4-54 (referred to as ANT and ANTSi treatment, henceforth) (FIG. 1A). SH-4-54 successfully blocks the phosphorylation of STAT3's tyrosine 705 residue in Ascl1-overexpressing MG and was not found to be toxic at the concentrations used for this study (FIG. 2). As previously described. ANT treatment resulted in GFP+MG-derived neurons that expressed bipolar neuron genes Otx2 and Cabp5, in addition to many MG that failed to undergo neurogenesis (FIG. 1B). When the STAT pathway inhibitor was co-injected with the TSA (ANTSi treatment), there was a striking increase in the number of MG-derived neurons (FIG. 1C, 1D). Quantification of the mature bipolar gene Cabp5 revealed a more than two-fold increase in the number of MG-derived neurons (24±5% and 52±3%, n=6 and n=8, for ANT and ANTSi treatments, respectively: FIG. 1E-F). All Cabp5+GFP+cells also expressed Otx2 and all Cabp5+cells had neuronal morphology; however, not all Otx2+cells expressed Cabp5 or had neuronal morphology, as previously described¹⁸.

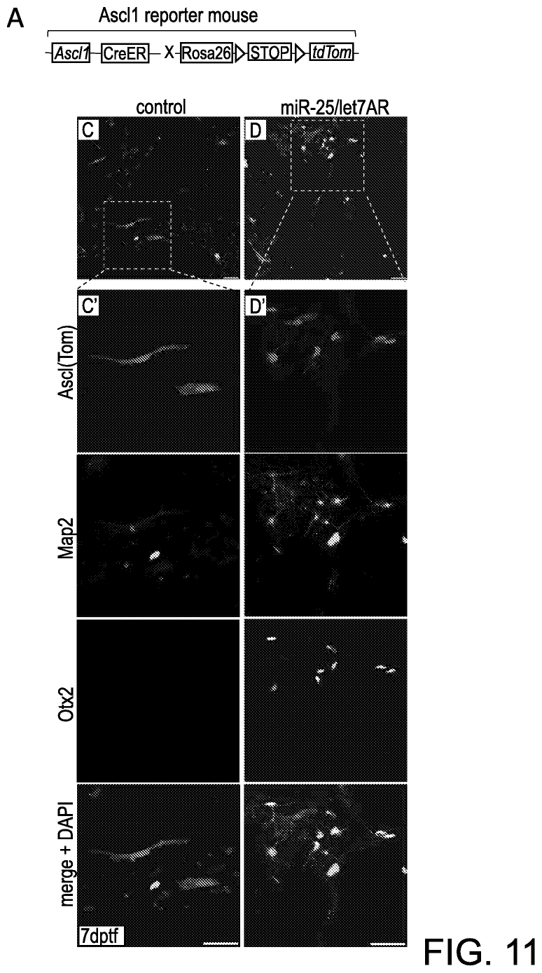

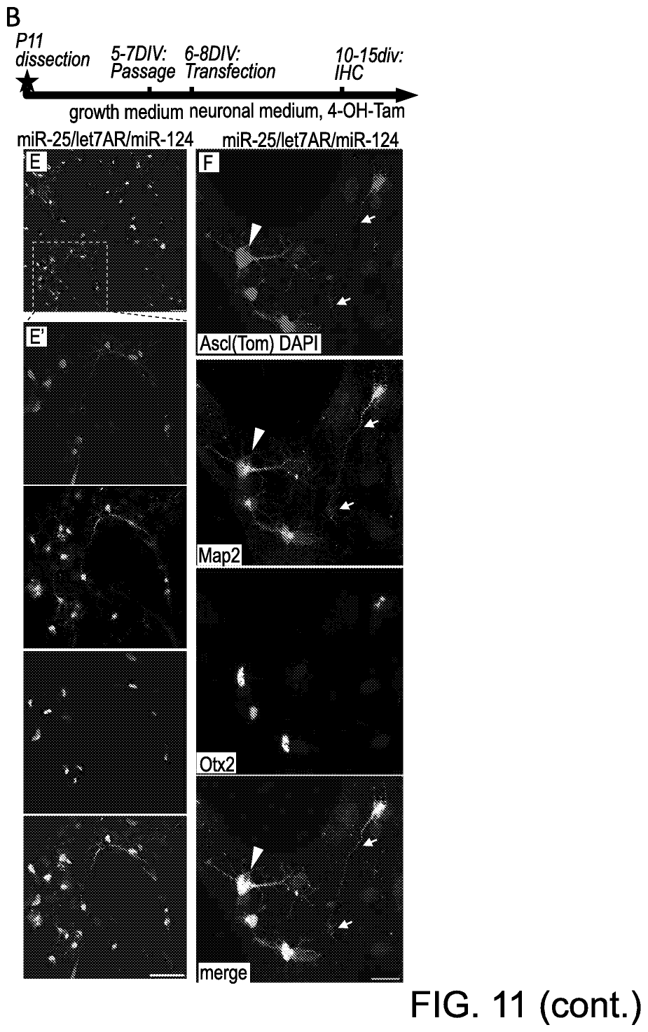

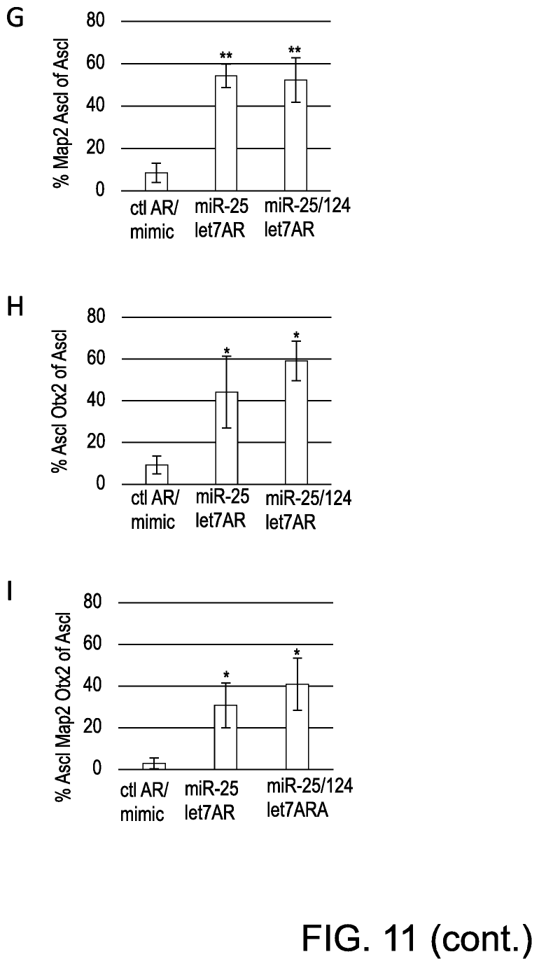

To determine whether the MG-derived neurons arise from mitotic proliferation or directly differentiate into neurons we intravitreally co-injected EdU with the NMDA on treatment day 8 and also with the TSA and STATi on treatment day 10 (FIG. 1G). Additionally, we performed twice daily IP injections of EdU from treatment day 9 through 12. After collecting the retinas on treatment day 26, we found that many of the MG and MG-derived neurons were labeled with EdU (FIG. 1H). Additionally, most of the MG-derived neurons labeled with EdU, and Cabp5, and had neuronal morphology (FIG. 11, arrows). These results show that many of the new neurons regenerated in the ANTSi condition are the result of mitotic proliferation of the MG. We found that approximately 53% of GFP+cells in the ANTSi condition were co-labeled with EdU.

ANTSi Treated MG-Derived Neurons Make Synaptic Connections with Existing Circuitry

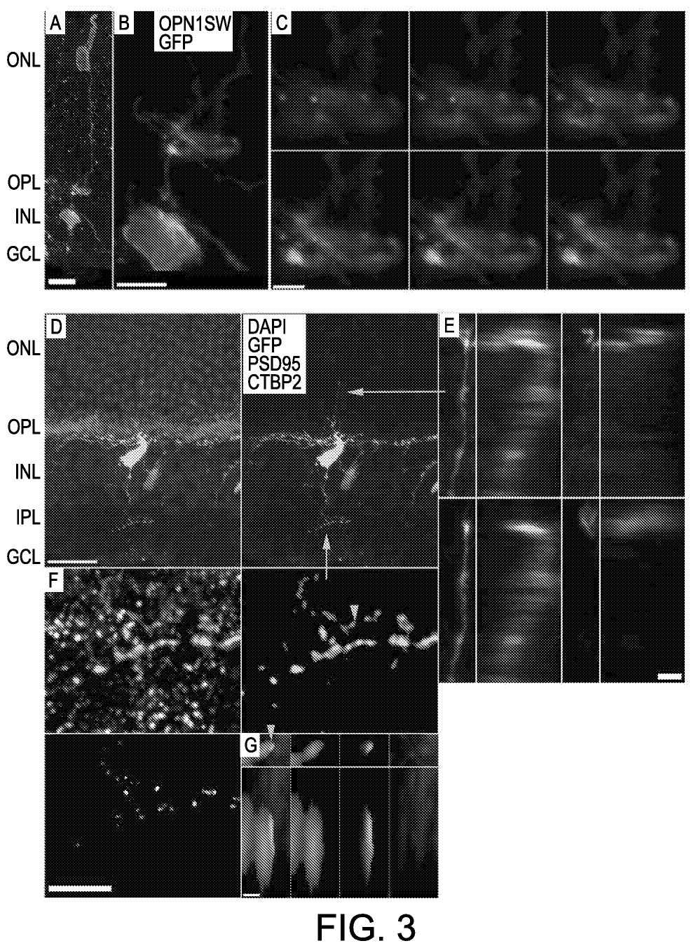

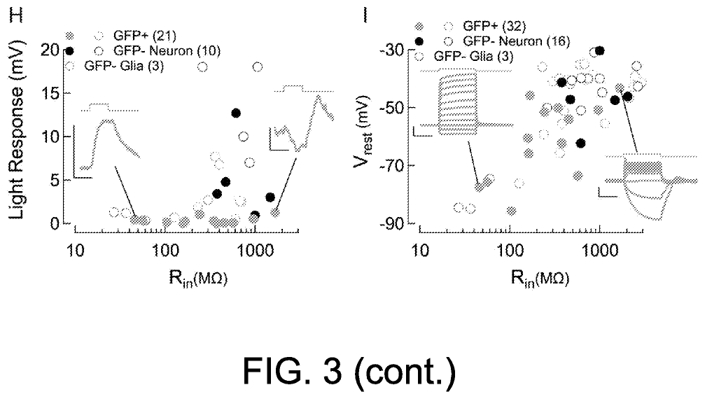

In our previous study, we found that MG-derived neurons generated by ANT treatment express synaptic proteins, make connections with cone terminals, and successfully integrate into the existing retinal circuitry and functionally respond to light stimuli¹⁶. In this study, we found that similar to ANT-treated neurons, ANTSi-treated MG-derived neurons express synapse proteins Ctbp2 and Psd95. Labeling retinal sections from the ANTSi treatment group for cone blue opsin (Opn1sw) showed MG-derived neurons contacting cone photoreceptors terminals in the OPL, indicative of photoreceptor-bipolar network connections (FIG. 3A-C).

To determine if ANTSi-treated MG-derived neurons express synaptic proteins in the correct cellular locations and form synaptic specializations with other cell types, we stained for Ctbp2 and Psd95 (FIG. 3D-G). In the OPL, we found Psd95+Ctbp2+photoreceptor synapses onto MG-derived neuron processes, indicative of a photoreceptor-to-MG-derived neuron synaptic specialization. Interestingly, we also found these contacts in the ONL (FIG. 3D-E) with photoreceptors synapsing onto the apical process of MG-derived neurons. This irregular location for photoreceptor synapse formation is likely due to the retraction of photoreceptor terminals²³. In the IPL, we found Ctbp2 puncta within the MG-derived neuron terminals (FIG. 33F-G). Pre-synaptic Ctbp2 within the MG-derived neurons was directly apposed to Psd95 from post-synaptic cell partners, indicative of a MG-derived neuron output onto other neurons in the IPL (FIG. 3G). Our data suggest that MG-derived neurons make synaptic specializations with photoreceptors in the OPL and post-synaptic cells in the IPL, consistent with our previous findings that MG-derived neurons are able to make synaptic connections within the existing retinal circuitry.

Next, we performed whole-cell electrophysiology recordings from GFP+ and GFP−cells to determine if ANTSi-treated MG-derived neurons have electrical properties consistent with retinal neurons. We plotted the population data from recorded cells onto a plot of previously recorded ANT-treated cells from another study16 (FIG. 3H-I). We found that GFP+cells had much higher input resistance (Rin) than GFP− MG and the resting membrane potentials (Vrest) of GFP+cells were depolarized compared to the GFP− MG. Most GFP+cells exhibited Rin and Vrest properties similar to GFP− neurons (FIG. 3I), suggesting that the GFP+cells' electrical properties were indeed more similar to retinal neurons. To determine if GFP+cells integrated into the retinal circuitry and received synaptic input from photoreceptors responding to light stimulus, we exposed dark adapted ANTSi-treated retinas to incremental increases of luminance and recorded from MG-derived neurons (FIG. 3H). Similar to our previous study of ANT-treated MG-derived neurons16, we found examples of ANTSi-treated MG-derived neurons responding to light increments by rapidly depolarizing, as would be expected for ON-preferring retinal neurons. Light responses presented with amplitudes ˜2 mV, which were not as robust as some of the cell recordings from our previous study; however, these recordings were performed on cells after 2, 5, and 7 weeks post-treatment, whereas our previous study included cells from later time points ranging from 4-15 weeks. Taken together, these findings indicate that MG derived neurons in the ANTSi treatment condition have retinal neuron electrical properties and receive synaptic input from photoreceptors resulting in light-evoked visual responses.

Single-Cell RNA-Seq on Reprogrammed Müller Glia.

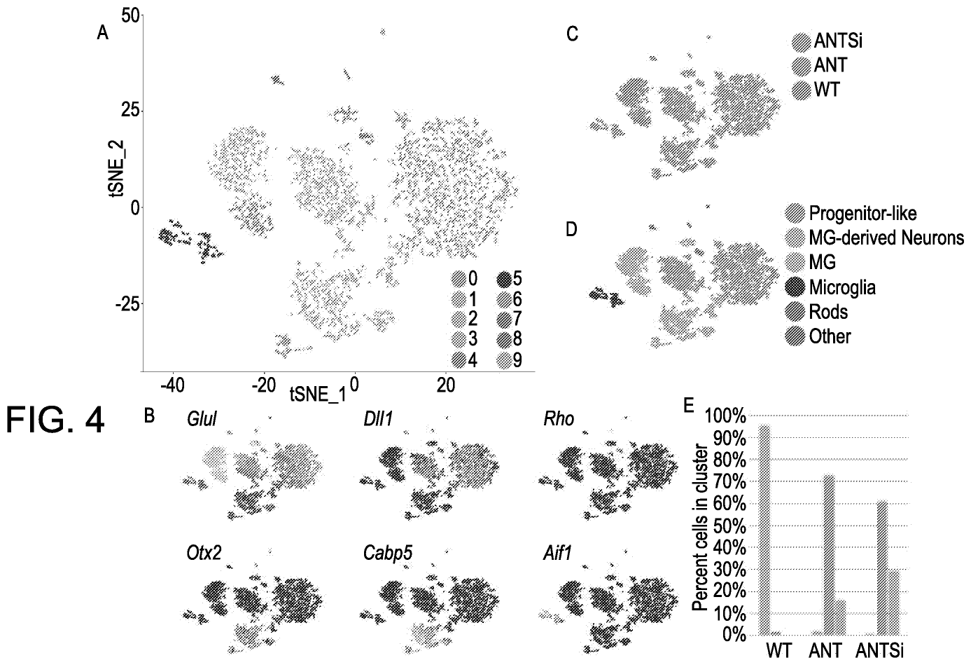

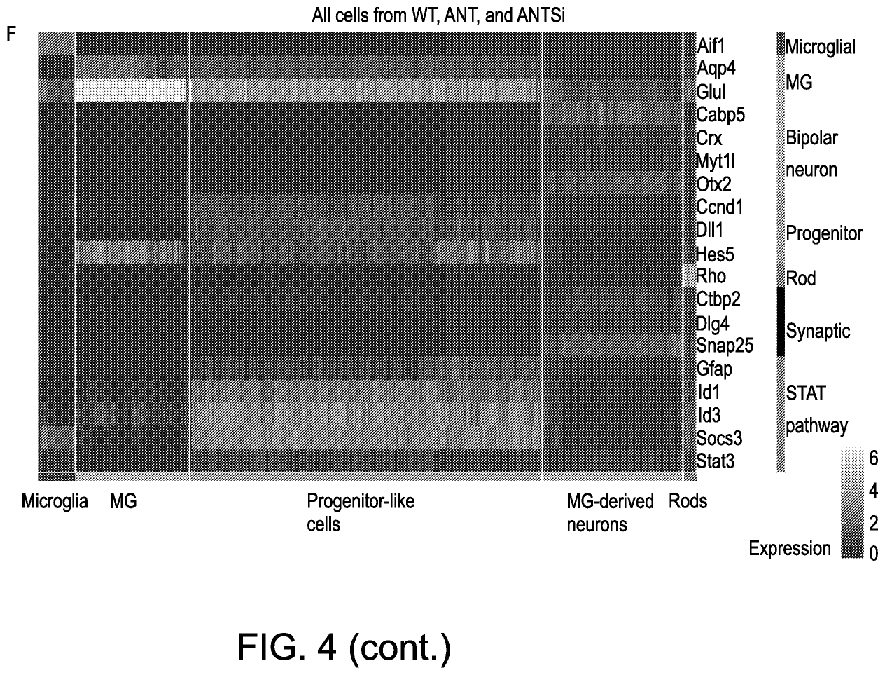

To further characterize the effects of STAT inhibition during MG reprogramming, we ran single-cell RNA-seq (scRNA-seq) on FACS-purified MG and MG-derived cells 14 days after ANTSi treatment, similar to that described in an earlier study¹⁶. In total 648, 823, and 2283 cells were analyzed from WT, ANT, and ANTSi treatments, respectively. All three (WT, ANT, and ANTSi) datasets were combined, normalized, scaled and analyzed in R with Seurat and Monocle²⁴⁻²⁶. Projecting all three treatments onto a single tSNE plot shows ten distinct clusters (FIG. 4A), differentially associated with the different treatment conditions (FIG. 4C). We identified the cell types comprising the clusters by their unique expression of specific known marker genes. Some of these genes are shown projected onto the individual clusters (FIG. 4B) and additional genes are shown associated with the clusters as a heatmap (FIG. 4F). For example, Clusters 3 and 4 were comprised of cells almost exclusively from the untreated (WT) condition and expressed genes normally present in MG, such as GluI and Aqp4: these were classified as “MG” clusters (FIG. 4D). Clusters 0 and 2 showed enrichment for progenitor genes (DII1; FIG. 4B) but lacked neuronal gene expression (Otx2 and Cabp5) and showed reduced glial gene expression (GluI). We therefore classified clusters 0 and 2 as “Progenitor-like” cells. Clusters 1 and 6 showed enrichment for neuronal genes and greatly reduced expression of glial genes and were classified as “MG-derived Neurons”. In addition to these major clusters, there were two contaminating cell populations that we do not think were derived from MG. These include microglial cells, identified by their expression of Aif1 (IBA1), and a small number of cells that express Rho and other photoreceptor genes that we consider contaminating “Rods”. These two populations are present in the FACS-purified cells regardless of treatment group, whereas the progenitor-like cells and MG-derived neurons are only present in the ANT and ANTSi conditions (FIG. 4C-D). Clusters 8 and 9 contained a small number of cells and were not enriched for any particular retinal cell type (FIG. 4D: “Other”).

The immunofluorescent analysis of retinas from the ANTSi condition indicated that a greater percentage of progenitor-like cells differentiate into neurons when compared with the ANT treatment. We analyzed the scRNA-seq results to determine whether a similar trend could be detected. Quantifying the number of WT, ANT, and ANTSi cells in the MG, progenitor-like, or MG-derived neuron clusters (FIG. 4E) revealed nearly double the number of MG-derived neurons in the ANTSi treatment compared to ANT (29% of total ANTSi cells vs. 16% of total ANT cells). The increase in the MG-derived neuron population in the ANTSi treatment was accompanied by a decrease in the number of progenitor-like cells. These data support the immunofluorescent analysis that STAT inhibition results in a population shift of MG from progenitor-like cells to MG-derived neurons and doubles reprogramming efficacy.

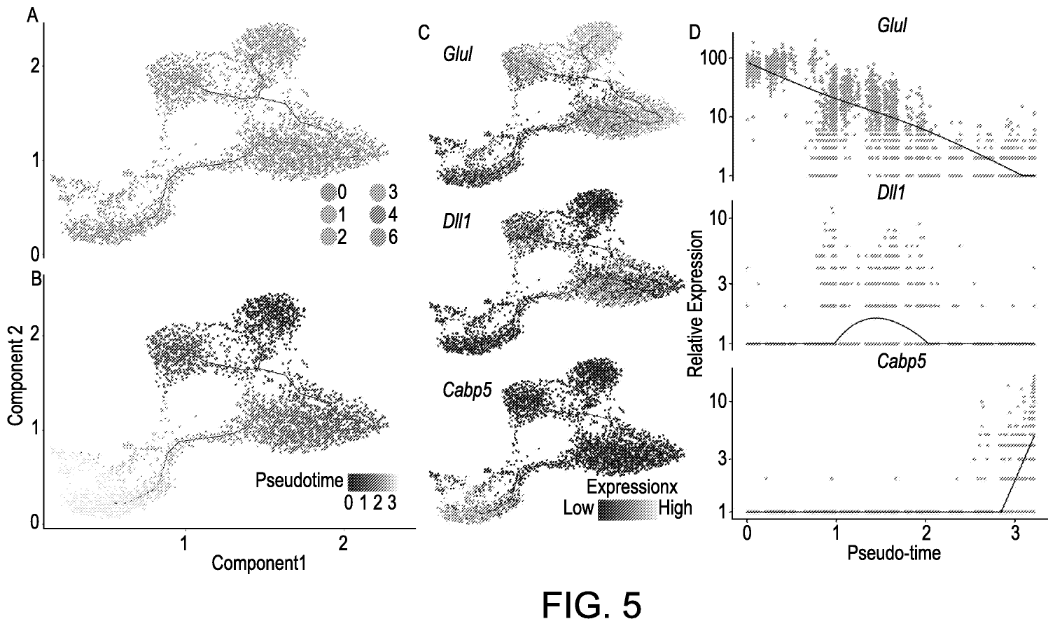

To better understand the trajectory of the reprogramming process, we used the Monocle analysis package on a subset of the data that only included MG, progenitor-like cells, and MG-derived neurons from clusters 0, 1, 2, 3, 4, and 6 (FIG. 5A). We found the pseudotime scale tracked with our classified cell types, with MG being the root state and transitioning through the progenitor-like cell state and eventually progressing towards MG-derived neurons (FIG. 5B). We observed cells decreasing expression of glial genes (e.g. GluI) as they progress towards progenitor-like cells and MG-derived neurons (FIGS. 5C-D). Cells in the progenitor-like clusters exclusively express progenitor genes, such as DII1 and as cells move towards the MG-derived neuronal clusters they upregulate neuronal genes, like Cabp5.

Although the trajectory analysis shows that MG progress to neurons through a progenitor-like state, it also suggests that this state is not identical in the ANT and ANTSi conditions. The progenitor-like cells in these two conditions cluster separately and although both clusters express progenitor genes, like DII1, the pseudotime plot suggests ANT reprogrammed cells are “closer” to MG than the cells from the ANTS treated retinas. This led us to hypothesize that progenitor cells may be kept in a stable, but non-neural state, by STAT pathway activation. Indeed, the progenitor-like cells highly express various STAT pathway target genes (e.g. Gfap, Id1, Id3, Socs3. FIG. 4F), while MG and MG-derived neurons have reduced or no expression of STAT targets.

ChIP-Seq for Ascl1 in P0 Mouse Retinal Progenitors and Reprogrammed Müller Glia.

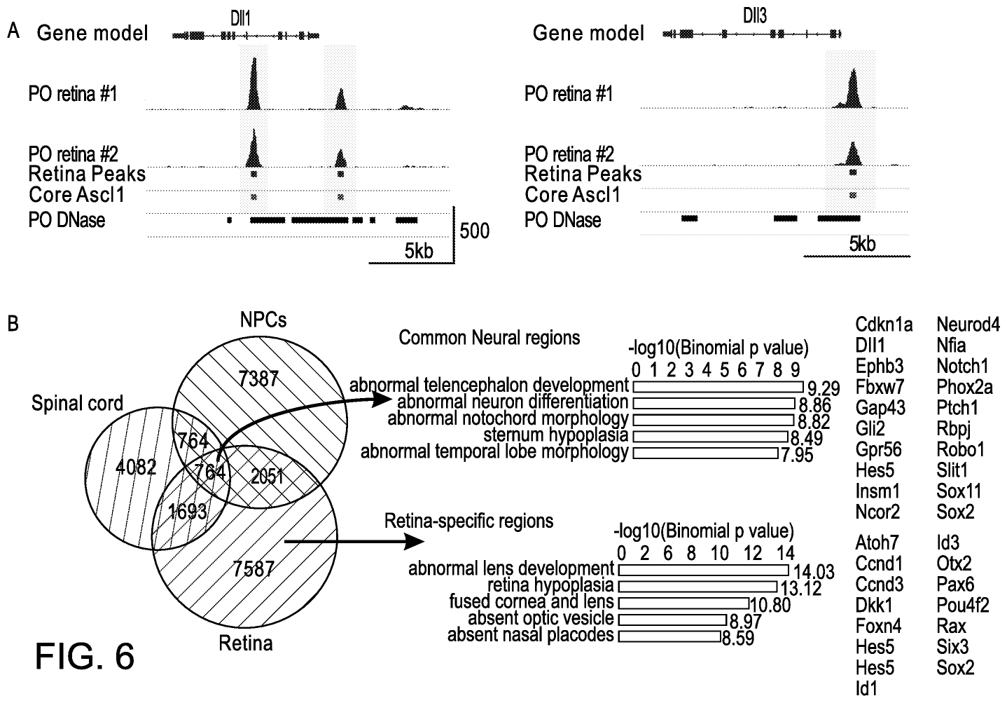

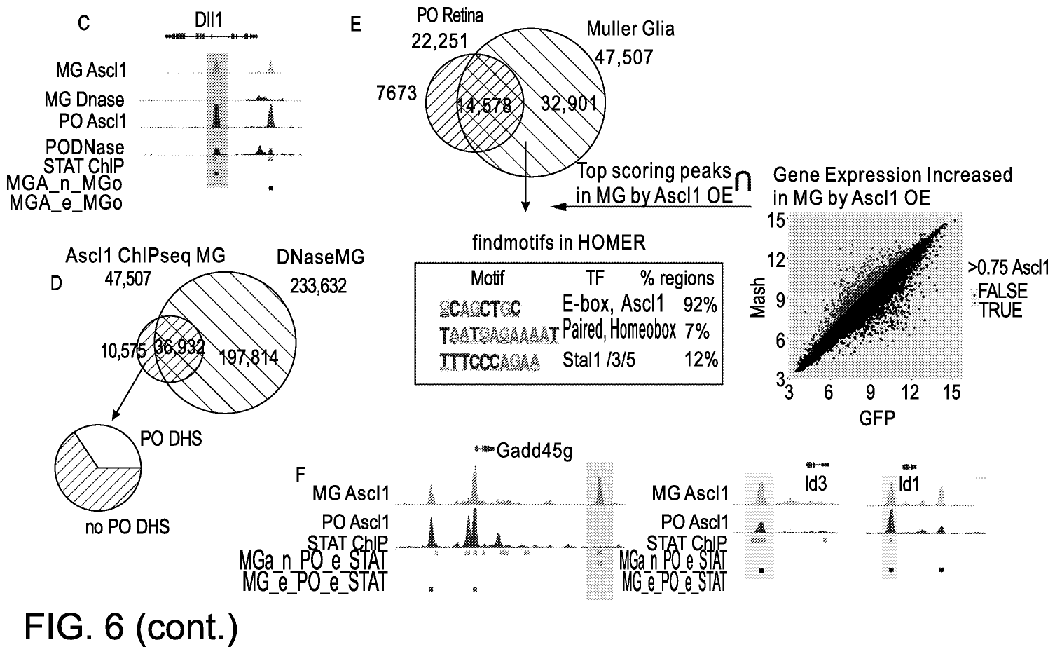

To explore potential mechanisms by which STAT inhibition promotes Ascl1-mediated retinal regeneration, we carried out ChIP-seq for Ascl1 in MG and compared this with retinal progenitors. In order to determine the endogenous binding pattern of Ascl1 in retinal progenitor cells on a genome-wide scale, we performed ChIP-seq for Ascl1 in P0 retina (FIG. 6A) and identified 22,251 peaks using HOMER²⁷ with a False Discovery Rate (FDR) of 0.1%. The top-scoring motif in these peaks (using MEME) was the canonical Ascl1 E-Box²⁸. The great majority of the Ascl1 peaks overlapped with an accessible chromatin region (FIG. 6; P0 DNase Track)². We compared the retinal Ascl1 peaks to previous ChIP-seq for Ascl1 in other neural tissues. For this analysis, we focused on the highest scoring peaks that replicated in two independent P0 retinal Ascl1-ChIP-seq runs (7587 sites, FIG. 6A). When the three-different neural Ascl1-ChIP-seq datasets were compared, we found that the majority of the Ascl1-bound regions in each type of neural tissue were unique to that tissue (FIG. 6B): for the peaks present in retina and another neural tissue, there was approximately the same degree of overlap for each of the combinations, while only a relatively small subset of peaks were common to all neural tissues (764; FIG. 6A-B: Core Ascl1 track). Gene Ontology analysis using the GREAT algorithm³⁰ showed that those peaks unique to retina were enriched for eye, optic and retinal GO terms (for mouse phenotype), while those regions common to all three neural tissues instead have enrichment for GO terms of non-retinal neural tissues (e.g. telencephalon; FIG. 6B). In addition, the genes associated with these GO terms for the retinal-specific regions were genes expressed more highly in retina than in other regions of the CNS, like cerebral cortex or spinal cord (e.g. Rax, Six3, Atoh7, Otx2; FIG. 6B), while those genes associated with the Ascl1-bound regions common to all three neural tissues were potentially representative of a more generic neural program of differentiation (Notch1. Insm1, and DII1), though it is important to note that there are also region-specific regulatory sites for common targets, like Hes5 and Sox2.

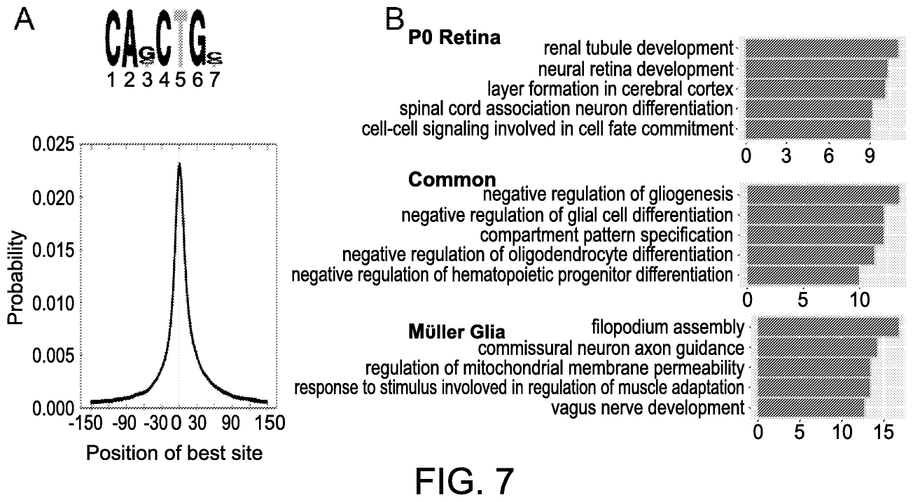

To determine the extent to which Ascl1 binds to similar regions in MG as in retinal progenitors, we performed ChIP-seq for Ascl1 in MG during the reprogramming process. Retinas from P12 mice (germline-rtTA: tetO-Ascl1) were dissociated and the MG were grown in culture. After 7 days, Ascl1 expression was induced in the purified MG by addition of doxycycline to the culture medium. Chromatin was then collected after 6 days post-Ascl1 induction and subsequently processed for Ascl1 ChIP-seq. The subsequent sequencing reads were filtered and mapped to the mouse genome (FIG. 6C) and peaks called using HOMER. In total, 47.507 peaks were called with a False Discovery Rate (FDR) of 0.1%; MEME analysis shows that the top-scoring motif was the canonical Ascl1 E-Box, similar to that described above for the P0 retina (FIG. 7A).

We performed a binding site overlap analysis between the peaks from the two cell populations. As shown in FIG. 6E, 31% (14,578/47,507) of Ascl1 binding regions in MG overlap an ‘appropriate’ Ascl1 binding site (i.e. one present in P0 retinal progenitor cells); however, the majority (˜70%) of the Ascl1 bound regions in MG are not present in the P0 retina. This discrepancy is not simply due to the higher number of binding sites in the MG, since 34% (7,673/22,251) of retinal progenitor Ascl1 binding sites are not bound in the MG. Therefore, although the majority of P0 retinal progenitor Ascl1 bound regions are also bound by Ascl1 in MG, there is substantial binding of Ascl1 to “inappropriate” sites in the genome of MG. In addition, there are also many potential cis-regulatory sites in progenitors that are not bound in the MG.

When we further analyzed the Ascl1 binding sites that are specific to retinal progenitors and compared them with those bound in the MG using the ‘GREAT’ gene ontology algorithm, the majority of top enriched terms related to neurogenesis (e.g.—‘neural retina development, layer formation in cerebral cortex’), suggesting that these Ascl1 binding sites are important for normal neuronal development of the retina and not dispensable binding events. Ascl1 binding sites that occurred in both progenitors and MG were enriched for neurogenic and gliogenic terms (e.g.—‘negative regulation of gliogenesis’, ‘negative regulation of oligodendrocyte differentiation’). However, the majority of Ascl1 binding sites specific to MG were not associated with retinal development (e.g.—‘filopodium assembly’, ‘regulation of mitochondrial membrane permeability’) (FIG. 7B). Therefore, while Ascl1 binds 66% of all appropriate sites in retinal progenitors, the majority of binding sites in MG are inappropriate and potentially not productive towards neurogenic reprogramming.

Studies in induced pluripotent cell reprogramming have shown that induction of pluripotency genes by Yamanaka factors³¹ (Oct4. Sox2, cMyc, and Klf4) is initially limited by the accessible chromatin sites in the somatic cell genome already bound by existing transcription factors. We wondered whether the inappropriate sites bound by Ascl1 in MG are due to opportunistic binding to DNase-Hypersensitive sites (DHSs) that are present in the MG and not in the retinal progenitors. Therefore, we quantified the overlap between Ascl1 ChIP-seq peaks and DHSs that are present in MG. As shown in FIG. 6D, 78% of Ascl1 binding sites occurred within DNase-hotspots in the MG and 22% occurred within non-hypersensitive chromatin. This result suggests that while most of the binding in MG occurs at sites that are already accessible, between one fifth and one quarter of the Ascl1-bound sites in MG were “pioneered” by Ascl1, consistent with prior results in other cells³³. We hypothesized that these pioneer Ascl1 binding events in MG were actually occurring at regions of DNase-hypersensitivity in the P0 retina. This scenario would be productive towards the goal of reprogramming the cis-regulatory landscape of MG towards that of retinal progenitors. Therefore, we quantified the overlap between pioneer Ascl1 binding sites in MG and P0 DNase-hotspots (FIG. 6D). We found that 34% (3591/10,575) of the pioneered sites overlap a P0 DNase-Hotspot. However, a much greater number, 66% (6984/10,575) of the pioneered sites, do not overlap a DNase-Hotspot in either P0 retina or MG, and thus are potentially non-productive pioneered sites.

The preceding analysis suggests that while a third of Ascl1 pioneering events are productive for reprogramming towards a retinal progenitor state, a majority of pioneering events appear to be unproductive towards reprogramming to the retinal progenitor state. We hypothesized that other transcription factors present in the MG might be collaborating with Ascl1 to direct it to these inappropriate³². To determine which factors might be associated with the inappropriate Ascl1 ChIP-seq peaks in MG (i.e. not present in progenitors), we used HOMER to identify sequence motifs enriched in these regions (for genes that increase >0.75 fold in reprogrammed MG, over P0 progenitors from our previous microarray³⁴). We found that Ascl1 was the top site followed by a generic homeodomain motif, but these regions were also significantly enriched for the STAT consensus site (FIG. 6E). Thus, the activation of the STAT pathway in the MG after retinal injury may direct Ascl1 to inappropriate sites on the DNA and thereby reduce its ability to reprogram MG more fully to progenitors/neurons.

Among the most highly upregulated genes in the Ascl1-reprogrammed progenitor-like cells include the Inhibitor of Differentiation genes (Id1, Id2. and Id3). These genes code for proteins that are similar to bHLH transcription factors, like Ascl1, except they lack a DNA-binding domain, and thus can form heterodimers with the bHLH family of transcription factors and prevent their ability to activate transcription^{35,36}. The scRNA-seq analysis shows that the Ascl1-overexpressing MG that failed to convert to neurons (and retained RPC-like gene expression) highly expressed Id genes Id1, Id2, and Id3 (FIG. 4F). Ascl1 ChIP-seq and DNase-seq from both P0 and MG show strong binding sites at accessible chromatin at both Id1 and Id3 promoters (FIG. 6F). Additionally, a previously generated STAT3 ChIP-seq dataset from oligodendrocytes in the brain (GEO: GSM2650746) indicated STAT3 binding sites in the same region as the Ascl1 binding sites (STAT ChIP track, FIG. 6F). Taken together, these findings suggest that STAT pathway activation and/or Ascl1 binding may induce Id gene expression, which inhibits Ascl1 from initiating neurogenesis in a subset of MG.

ChIP-Seq for Ascl1 in Control and STATi-Treated Reprogrammed Müller Glia.

To directly assess the effect that STAT pathway activation has on Ascl1 binding at Id genes and more broadly across the genome during MG reprogramming, we performed an Ascl1 ChIP-seq on control and STATi-treated MG. Frozen stocks of MG from P12 germline-rtTA:tetO429 Ascl1 mice were thawed and cultured to confluency for 2 days in high FBS growth media. Cells were then given a low FBS media with STATi or vehicle for the remaining 4 DIV. At 3 DIV (1 day after STATi), doxycyline was administered to induce Ascl1 overexpression in the MG. Cells were then collected and fixed for ChIP-seq after 6 DIV. FASTQ files were aligned to the mm10 genome using Bowtie2 and peaks were called using MACS2. We observed 106,063 and 89,793 high confidence peaks in the Ascl1 control and Ascl1/STATi-treated samples, respectively. The majority of peaks overlapped between the two samples, with 80,169 sites being common to both treatment conditions. Both datasets contained the canonical Ascl1 E-box as the top scoring motif, as determined by HOMER, with strong central enrichment of peaks from both datasets around the Ascl1 motif.

Based on our findings that inappropriate Ascl11 binding sites were frequently found with the STAT motif, we hypothesized that inhibition of the STAT signal would reduce Ascl1 recruitment to these sites. To identify genes that might be regulated by STAT signaling in the MG during reprogramming with Ascl1, we performed a differential analysis between the control Ascl1 and Ascl1/STATi datasets using edgeR37,38. Individual peaks were compared between the two datasets and plotted as a MA plot. Ascl1 ChIP-seq peaks that were significantly reduced in the STATi treatment condition were present at various STAT pathway target genes (e.g. Bcl2, Pias1, Stat5a/b), and Id1 and Id3 were among these. Decreased Ascl1 binding was observed at STAT3 bound regions near Id1 and Id3 after STATi treatment, with significantly reduced peaks. Together, these findings add further support to the hypothesis that STAT signaling may redirect Ascl1 to non-productive cis-regulatory regions, and in some cases, inhibitors of neural reprogramming, like Id1, Id3 and Hes5.

In addition to reducing the binding of Ascl1 to STAT target regions in MG, inhibition of the STAT pathway leads to increases in Ascl1 binding to non-STAT targets. Ascl1 ChIP-seq peaks that were significantly enriched in the STATi treatment condition were present at neuronal genes associated with amacrine and bipolar cell fates (e.g. Elavl4, Otx2, Isl1, and Prox1) as well as synaptic genes (e.g. DIg4 and Snap25), consistent with our observations that this treatment leads to an increase in neuronal differentiation. By contrast, peaks located near genes associated with progenitor cells were also significantly decreased in the STATi treatment condition (e.g. Ccnd1 and Ccnd3). Thus, STATi results in enrichment of Ascl1 binding at neuronal genes, suggesting that inhibiting STAT pathway activation during reprogramming leads to more productive binding of Ascl1 at genes necessary for neurogenesis.

Id Genes are Dysregulated in the Presence of Ascl1

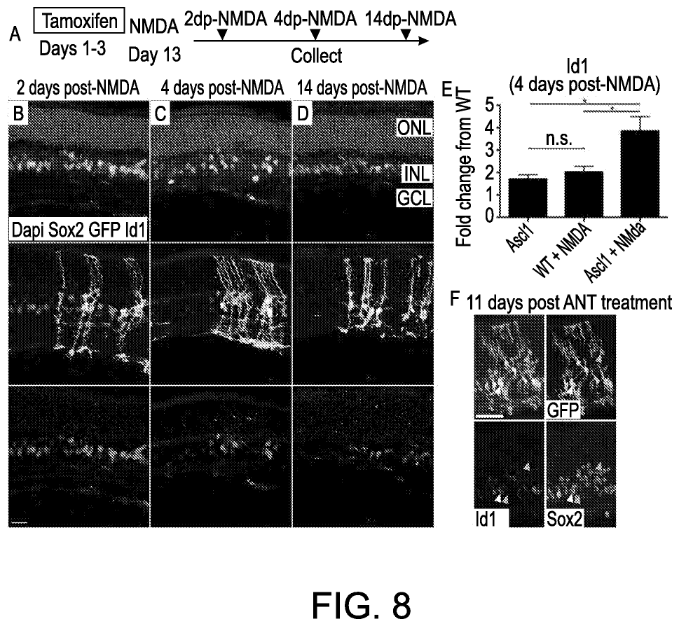

To determine the effects of Ascl1 overexpression and damage on STAT pathway activation and Id1 expression, we performed a NMDA time course (FIG. 8A). Under normal conditions, in WT animals Id1 is not present at detectable levels in MG (FIG. 7A). At 1 day and 2 days post-NMDA, Id1 is highly expressed in MG but begins to decline at 4 days and returns to basal levels by 9 days post-NMDA (FIG. 7B-F). In the Ascl1-overexpressing mice, all MG express Id1 at similarly high levels by 2 days post-NMDA; both the Ascl1-overexpressing GFP+MG, and the non-Ascl1-overexpressing GFP− Sox2+MG (FIG. 8B). By 4 days post-NMDA damage, Id1 was found to be at lower levels in the GFP− MG and highly expressed in the Ascl1 GFP+MG (FIG. 8C). Eleven days after TSA treatment, the GFP+cells that show signs of neuronal morphology and downregulation of Sox2 have basal levels of Id1 expression (FIG. 8F, arrows). By 14 days post-NMDA damage, high Id1 expression became restricted to the MG that express Ascl1 (the GFP+MG) (FIG. 8D). Additionally, we performed RT-qPCR for Id1 on whole retinas taken from undamaged WT and Ascl1 overexpressing retinas as well as NMDA treated retinas (FIG. 8E, n=4 mice per group). Ascl1 overexpressing retinas treated with NMDA expressed significantly more Id1 at 4 days post-NMDA than all other treatment groups. Interestingly, the Ascl1-overexpressing retinas have similar levels of Id1 at baseline as the WT mice treated with NMDA. Taken together, these findings suggest that the normally transiently activated target genes of STAT become constitutively active in the presence of Ascl1 overexpression. Constitutive expression of ID proteins in Ascl1 expressing MG likely results in the ID proteins binding Ascl1, antagonizing the pro-neurogenic effects of Ascl1^{35,36}. Further evidence supporting this hypothesis is the fact that ANT-treated MG-derived neurons do not have any detectable Id1 expression (FIG. 8F, arrows), while the adjacent non-reprogrammed MG do have Id1 expression (white arrow).

Discussion

The findings in this Example highlight several important features of the reprogramming of glia to neuronal progenitors and neurons. The Ascl1 ChIP-seq and DNase-seq data reveal that the reprogrammed MG show similar Ascl1 binding and chromatin accessibility as the newborn mouse RPCs. However, the reprogrammed MG also have new Ascl1 peaks that are not normally found during development in RPCs, sites we label as “inappropriate.” A significant number of these inappropriate Ascl1 binding sites are found at genes that are not relevant to retinal development and are associated with an increase in expression of these genes in the reprogrammed MG. We further identified a consensus DNA binding motif for STAT3 that was significantly associated with these inappropriate Ascl1 peaks. Potential STAT targets that could underlie the effects we observe, include Id1 and Id3, dominant negative regulators of bHLH gene function. Lastly, this study provides the first evidence combining lineage tracing with EdU-labeling to demonstrate new neurons can arise from proliferating MG in an adult mammal.

One potential target of STAT signaling that could limit the regenerative response in mammalian MG is Id1. Following injury to the retina, the STAT target gene Id1 is transiently expressed in MG, but Id1 returns to basal levels within a week. We found that in Ascl1-overexpressing mice, Id1 expression is maintained in the MG, possibly maintaining the cells in a progenitor-like state and preventing them from generating new neurons. ID proteins are known to form dimers with class I bHLH proteins and inhibit their dimerization with class II bHLH factors to inhibit their transcriptional activation activity^{35,36}. Additionally, ID proteins can bind directly with HES proteins to maintain neural progenitors in an undifferentiated state³⁷⁻³⁸. ID proteins have also been shown to play an important role in other organ systems, such as the pancreas, where they can bind Hes1 and participate in the dedifferentiation and fate switching of exocrine cells to endocrine fates⁴⁰. JAK/STAT signaling is known to bias progenitors towards a glial fate in other regions of the nervous system as well⁴¹⁻⁴³. For example, inhibition of the JAK/STAT co-receptor gp130 in developing cortical progenitors has been found to increase their neurogenic production at the expense of gliogenesis⁴⁴. Thus, it is possible that STAT activation in astrocytes in other areas of the nervous system may limit their neurogenic potential via ID proteins in a manner similar to that we have described in the retinal MG.

Previous studies have demonstrated the importance of the source cell's epigenetic landscape and presence of endogenous transcription factors of that source cell during reprogramming^{32,33}. Fibroblast reprogramming to induced neuronal cells show that the newly generated cell types still retain their source cell signature. We previously found this to be the case in MG reprograming as well¹⁶, where the MG-derived neurons still express low levels of glial genes such as GluI or Aqp4. The present study demonstrates the importance of injury induced STAT pathway activation during reprogramming and shows a successful combinatorial analysis using epigenetic and gene expression datasets to confirm inappropriate transcription factor binding associated with non-productive gene expression.

Methods

Experimental Model and Subject Details

Mice

Glast-CreER:LNL-tTA:tetO-mAscl1-ires-GFP mice and rtTA germline: tetO-mAscl1-ires-GFP mice used in this study were from a mixed background of C57BL/6, B6SJLF1, and other backgrounds present at The Jackson Laboratory. Mice of both sexes were used in this study. For in vivo experiments, adult mice over the age of postnatal day 40 were used. Mice were housed in the specific-pathogen-free (SPF) animal facility at the University of Washington, Seattle, Wash. Mice were housed under controlled conditions. Mice underwent no previous treatments prior to testing. All procedures performed in this study were approved by the Institutional Animal Care and Use Committee at the University of Washington, Seattle.

Primary Cell Culture

Postnatal day 0, 11/12 retinas of both sexes from rTA germline:tetO-Ascl1-ires-GFP mice were digested with papain/DNase to single cells and MG were grown in culture as previously described^{34,45}. In brief, retinas were placed in a papain solution with 180 units/mL DNase (Worthington) and incubated at 37° C. for 10 min. Cells were triturated and added to an equal ovomucoid (Worthington) volume then spun down at 300 g at 4° C. to pellet. Cells were then resuspended in Neurobasal with 10% FBS (Clontech), mEGF (100 ng/mL; R&D Systems), 1 mM L-glutamine (Invitrogen), N2 (Invitrogen), and 1% Penicillin-Streptomycin (Invitrogen) with two retinas plated per 10 cm² at 37° C. Media was changed every 2 days until confluent monolayers of MG were passaged after 7 DIV and doxycycline was added to induce expression of Ascl1.

Method Details

Animals: All mice were housed at the University of Washington. All experiments and protocols were approved by the University of Washington's Institutional Animal Care and Use Committee. Adult mice used for in vivo experiments were Glast-CreER:LNL-tTA:tetO-mAscl1-ires-GFP mice and have been previously described¹⁸. The Glast-CreER and LNL-tTA mice were from Jackson Labs and the tetO-mAscl1-ires-GFP mice were a gift from M. Nakafuku (University of Cincinnati). The rtTA germline: tetO-mAscl1-ires-GFP mice for in vitro experiments were generated by crossing Nakafuku's tetO-mAscl1-ires-GFP mice onto the germline rtTA mice from Jackson Labs. Mice of both sexes were used in this study and adult mice were treated at ages comparable to our previously described study¹⁸.

Ascl1 Chromatin Immunoprecipitation-Sequencing (ChIP-Seq): P0 retinas or cultured, post-natal day 12, Müller glia (+/−Ascl1 overexpression, rtTA germline:tetO-Ascl1-ires-GFP mice ±doxycycline) were digested with papain/DNase to single cells and fixed with 0.75% formaldehyde for 10 minutes at room temperature. Sonication was performed with a probe sonicator (Fisher Scientific): 12 pulses, 100 J/pulse, Amplitude: 45, 45 seconds cooling at 4° C. between pulses. Immunoprecipitation performed with 40 μL anti-mouse IgG magnetic beads (Invitrogen Cat: 110.31) and 4 μg mouse anti-MASH1 antibody (BD Pharmingen Cat: 556604) or 4 μg mouse IgG against chromatin from 5 million cells per condition according to Diagenode LowCell Number Kit using IP and Wash buffers as described in²⁸. Libraries were prepared with standard Illumina adaptors and sequenced to an approximate depth of 36 million reads each. Sequence reads (36 bp) were mapped to the mouse mm9 genome using bwa (v 0.7.12-r1039). Merging and sorting of sequencing reads from different lanes was performed with SAMtools (v1.2). The HOMER software suite was used to determine and score peak calls (‘findPeaks’ function, v4.7) as well as motif enrichment (‘findMotifs’ function, v4.7, using repeat mask). Peak overlap analyses were performed using Bedops. For STATi and control Ascl1 ChIP-seq, reads were aligned to the mm10 genome using Bowtie2. The .sam files were converted to sorted .bam files using SAMtools. MACS2 was used to call peaks with default settings using the broad peaks annotation. Peak overlap analyses were performed using Bedops. The control Ascl1 ChIP-seq .bam file was downsampled by a factor of 0.69 to normalize the number of mapped reads over the common peaks found between treatment and control samples. This downsampled .bam file was used for all analyses. Differential accessibility analysis in Ascl1 ChIP-seq peaks was determined using edgeR as detailed in the edgeR user guide.

DNase I Hypersensititivy-Sequencing (DNase-Seq): Detailed protocols can be found at encodeproject.org. In brief: nuclei from retina were isolated using 25 strokes of a dounce homogenizer, tight pestle, in 3 mL homogenization buffer (20 mM tricine, 25 mM D-sucrose, 15 mM NaCl, 60 mM KCl, 2 mM MgCl₂, 0.5 mM spermidine, pH 7.8) and filtered through a 100 μm filter and washed with Buffer A (15 mM Tris-HCl, 15 mM NaCl, 60 mM KCl, 1 mM EDTA, 0.5 mM EGTA, 0.5 mM spermidine). Nuclei from Müller glia were isolated using TrypLE (Thermofisher) to obtain single cells, followed by incubation with 0.04% IGEPAL in Buffer A for 10 minutes at 4° C. Nuclei were incubated at 37° C. for 3 minutes in limiting concentrations of DNaseI enzyme in Buffer A with calcium supplement. The reaction was stopped using equal volume of Stop Buffer (50 mM Tris-HCl, 100 mM NaCl, 0.1% SDS, 100 mM EDTA, 1 mM spermidine, 0.5 spermine pH 8.0) and subsequently treated with proteinase K and RNase A at 55° C. Small (<750 bp) DNA fragments were isolated by sucrose ultracentrifugation and end repaired and ligated with Illumina compatible adaptors. Sequence reads were mapped to mm9 using bowtie (v 0.12.7) and DNaseI peak calling performed with Hotspot (http://www.uwencode.org/trohotspot/).

Electrophysiology: Recordings were performed under identical conditions to our previous study¹. All mice underwent dark-adaptation prior to euthanasia. Retinas were then removed, dissected, embedded in agar, and cut into 200 μm thick slices under infrared visualization. All prep was performed in Ames medium at 32-34° C. and oxygenated with 95% O2/5% CO2. Slices were placed under the microscope and perfused with oxygenated Ames at a rate of ˜8 mL per minute. Cells were targeted for recording using video DIC with infrared light (>950 nm), two-photon (I=980 nm). or confocal (I=488 nm) microscopy. Cells targeted for light responses under infrared conditions were exposed to full-field illumination via blue and green LEDs from a customized condenser. Cells targeted with the 488 nm laser were predominantly used to record Rin and Vrest properties. Recordings were performed using pulled glass pipettes (5-6 MW) and filled with solution containing (in mM): 123 K-aspartate, 10 HEPES, 1 MgCl2, 10 KCl, 1 Cacl2, 2 EGTA, 0.5 Tris-GTP, 4 Mg-ATP, and 0.1 Alexa-594 hydrazide.

Fluorescence-activated cell sorting (FACS): After euthanasia of mice, eyes were removed and retina dissected out and isolated from vitreous and retinal pigmented epithelium. Retinas were then dissociated in a Papain and DNase I solution for 20 minutes at 37° C. on a nutator. After incubation, retinas were gently triturated to generate a single-cell suspension, followed by the addition of Ovomucoid. Cells were then spun down at 300 g at 4° C. then resuspended in Neurobasal medium, passed through a 35 μm filter, and transferred to a BSA coated tube prior to FACS-purification. FACS was performed on a BD FACSAria III Cell Sorter (BD Biosciences) and the GFP-positive fraction was collected for single-cell RNA-seq. 44,000 cells in total were captured for single-cell RNA-seq.

Immunohistochemistry (IHC): Upon euthanasia of adult mice, eyes were removed and comeas dissected away. Globes were then fixed for 1 hour in 4% PFA in PBS followed by an overnight incubation in 30% sucrose in PBS at 4° C. Fixed eyes were then frozen in O.T.C. (Sakura Finetek) at −80° C. until sectioned. Frozen eyes were sectioned on a cryostat (Leica) at 16-18 μm thick and were stored at −20° C. until staining. All washes were 3 times 20 minutes in PBS on a rotating plate at room temperature. Slides were washed then blocked in 10% normal horse serum, 0.5% Triton X-100 in PBS for a minimum of 1 hour prior to primary. All primary antibodies were diluted in blocking solution and applied to tissue for a minimum of 3 hours. Slides were then washed and incubated with secondary antibodies for a minimum of 2 hours, which were diluted in PBS. Slides were then washed and cover slipped with Fluoromount-G (SouthernBiotech). Primary antibodies used were: rabbit anti-Cabp5 (a gift from F. Haeseleer. 1:500), rabbit anti-PSD95 (Abcam, 1:100, Ab-18258-100), mouse anti-Ctbp2 (BD Biosciences. 1:1000, 612044), chicken anti-GFP (Abcam, 1:500, Ab13970), goat anti-Otx2 (R&D Systems, 1:100, BAF1979), goat anti-Sox2 (Santa Cruz, 1:100, SC-17320), rabbit anti-Opn1sw (Millipore, 1:300, AB5407), rabbit anti-Id1 (Biocheck. 1:1000, BCH-1/#37-2). Secondary antibodies used were all donkey anti-species (Life Technologies) and were diluted 1:300 with a 1:100,000 DAPI (Sigma) in PBS and were applied in dark conditions. TUNEL staining was performed using the Promega TUNEL kit. EdU-labelling was performed using the Thermo Fischer Scientific Click-iT EdU system.

Injections: Intraperitoneal injections of tamoxifen were administered to adult mice for up to five consecutive days to induce expression of the tetO-mAscl1-ires-GFP gene. Tamoxifen was administered at a concentration of 1.5 mg per 100 μL of corn oil. Intravitreal injections of NMDA were administered at a concentration of 100 mM in PBS at a volume of 1.5 μL. Intravitreal injections of TSA (Sigma) were administered at a concentration of 1 μg per μL in DMSO at a volume of 1.5 μL. Intravitreal injections of SH-4-54 STAT-inhibitor (Selleck Chem) were administered at a concentration of 10 mM in TSA containing DMSO at a volume of 1.5 μL. All intravitreal injections were performed on isoflurane-anesthetized mice using a 32-gauge Hamilton syringe. For EdU-labelling experiments, NMDA and EdU were intravitreally co-injected in a mixture containing 1 μL of 34 mM NMDA, 0.5 μL EdU (5 mg/mL), and 0.5 μL PBS at a volume of 2 μL. Two days later the TSA, STATi, and EdU were intravitreally co-injected in a mixture containing 1 μL TSA (2 mg/mL), 0.5 μL EdU (5 mg/mL), and 0.5 μL SH-4-54 (25 mg/mL) at a volume of 2 μL. From treatment days 9 through 12, EdU was intraperitoneally injected twice daily for a total of 8 injections at a concentration of 1 mg/mL EdU at a volume of 100 uL in PBS.

Microscopy/cell counts: All images were taken on a Zeiss LSM880 confocal microscope. For cell counts, all images were taken at the same magnification and a minimum of 4 fields per retina were captured as a Z-stack then analyzed in ImageJ (NIH, Bethesda, Md.). To be counted as a positive cell, the marker of interest needed to be viewed in at least 3 μlanes of the Z-stack to ensure accurate co-localization. Counts were summed together for a single retina then percent co-localization was calculated per retina. For the Id1, Sox2, GFP staining figure, all images were captured at the same magnification, laser settings, and detector settings. In addition, all sections were stained at the same time in the same manner to show comparable relative protein expression in each treatment. For synaptic staining images, the Airyscan detector on the Zeiss LSM880 was used to capture Z-stacks and maximize resolution and co-localization of synaptic proteins. Airyscan images were then processed in Amira image software (FEI). The GFP channel was masked and Psd95 that was within this GFP mask in the OPL was displayed to highlight Müller glial-derived neurons synaptic input. Similarly, the GFP channel was masked and Ctbp2 that was within this GFP mask in the IPL was displayed to highlight Müller glial-derived neurons synaptic output.

Quantitative reverse transcription PCR (RT-qPCR): Glast-CreER:LNL-tTA:tetO-mAscl1-ires-GFP mice received 3 days of tamoxifen injections to induce Ascl1 expression on treatment days 1 through 3. On treatment day 13, WT and Ascl1 expressing mice received an intravitreal injection of 100 mM NMDA in 1.5 μL volume. Four days after NMDA administration WT and Ascl1 expressing mice were euthanized and retinas collected and digested in TRizol reagent (Thermo Fischer). RNA was extracted and collected in miRNeasy Mini Kit columns in accordance with manufacturer instructions (Qiagen). Reverse transcription was performed on 1 g of purified RNA using the iScript Reverse Transcription Supermix kit (Bio-Rad). The cDNA was then added to SsoFast (Bio-Rad) for qPCR. A total of 4 biological replicates were run for each condition (WT, WT+NMDA. Ascl1, Ascl1+NMDA) and each biological replicate was run in triplicate. Id1 cycles were subtracted from housekeeping gene Gapdh (ΔCt) and then subtracted from WT (ΔΔCt) to determine fold change 2^{(-ΔΔCt)}. Primers for Gapdh were (5′-GGCATTGCTCTCAATGACAA-3′ (SEQ ID NO: 29) and 5′-CTTGCTCAGTGTCCTTGCTG-3′ (SEQ ID NO: 30) and primers used for Id1 were (5′-TACGACATGAACGGCTGCTACTCA-3′ (SEQ ID NO: 31) and 5′-TTACATGCTGCAGGATCTCCACCT-3′ (SEQ ID NO: 32)).

Single-cell RNA-sequencing (Single-cell RNA-seq): Four ANTSi-treated mice (8 eyes) had their retinas pooled for FACS-purified cells and were spun down at 300 g at 4° C. then resuspended at a concentration of 1000 cells per μL in a 0.04% BSA in PBS solution. Cells were processed through the 10× Genomics Single Cell 3′ Chip and processed through the standard Chromium Single Cell 3′ Reagent Kits User Guide protocol with a target capture of 4,000 cells. Library QC was determined by Bioanalyzer (Agilent). Libraries were then sequenced on Illumina NextSeq 500/550 vs kit and reads were processed through 10× Genomics Cell Ranger pipeline. Reads were aligned to the mm10 genome and the filtered output files from Cell Ranger were processed in R using tools in the Seurat package. For Seurat analyses, the newly generated single-cell RNA-seq dataset from ANTSi-treated mice were compared with our previously generated WT and ANT-treated datasets¹⁶.

Western Blot/Analysis: Retinas from P11 rtTA germline: tetO-mAscl1-ires-GFP mice were dissociated and MG were grown in culture as previously described^{34,45}. Confluent monolayers of MG were passaged and doxycycline (1:500) was added to the media to overexpress Ascl1 for 6 days, and a subset of cultures received SH-4-54 STAT-inhibitor (Selleck Chem) on the fifth day of treatment for 24 hours. MG cultures were lysed with buffer containing 25 mM Tris-HCl pH 7.5, 150 mM NaCl, 1 mM EDTA, 1% Triton X-100, 5% glycerol, 1× protease inhibitor cocktail, and 1× phosphatase inhibitor cocktail and equal amounts of protein samples were loaded and run in a 4% to 20% SDS gel (Bio-Rad Laboratories). Protein was transferred to a polyvinylidene fluoride membrane (Thermo Fisher Scientific, Waltham, Mass., USA), blocked (5% BSA and 0.1% Tween 20 in 1×TBS) for at least 1 hour at room temperature and stained with primary antibodies (Phospho-STAT3 Y705 and STAT3, R&D Systems 9131S and 9132S) diluted in blocking solution overnight at 4° C. Membranes were washed with 0.1% Tween 20 in 1×TBS and then incubated with HRP-conjugated secondaries (Bio-Rad Laboratories) diluted in blocking solution for 1 hour at room temperature. Signals were visualized on X-ray film with a commercial substrate (SuperSignal West Dura Extended Duration Substrate; Thermo Fisher Scientific) and quantified using ImageJ software.

Quantification and Statistical Analysis

Ascl1 ChIP-Seq:

The HOMER software suite was used to determine and score peak calls (‘findPeaks’ function, v4.7) as well as motif enrichment (‘findMotifs’ function, v4.7, using repeat mask), Peak overlap analyses were performed using Bedops. Gene Ontology analyses of Ascl1 ChIP-seq peaks were generated using GREAT algorithm.

Immunohistochemistry:

For Otx2 quantification, retinas from 16 ANT-treated mice and 13 ANTSi-treated mice were analyzed. For Cabp5 quantification, retinas from 6 ANT-treated mice and 8 ANTSi-treated mice were analyzed. An unpaired t-test was performed using Graphpad Prism for each figure. Data is presented as mean±SEM.

RT-qPCR:

Id1 Expression was Performed on 4 Biological Replicates from Each Condition

Single-Cell RNA-Seq:

Quantification of the percent cells in each cluster was performed in R using the Seurat toolkit. Cells that were located in either the Müller glial, Progenitor-like, or MG-derived neuron clusters were identified by which treatment they came from (WT. ANT, ANTSi). The total number of cells in each cluster from each treatment was then divided by the total number of cells from that corresponding treatment to generate percentages, which are shown in the bar graph. The heatmap was created using the doHeatmap function from the Seurat toolkit in R. The intensity of the log 2 expression is shown.

Western Blot:

Quantification of gel bands was performed using ImageJ.

### REFERENCES

1 Fischer. A. J. & Reh, T. A. Nat Neurosci 4.247-252, doi:10.1038/85090 (2001).
2 Raymond, P. A., et al. BMC Dev Biol 6, 36, doi:10.1186/1471-213X-6-36 (2006).
3 Fausett, B. V. & Goldman, D. J Neurosci 26, 6303-6313, doi:10.1523/JNEUROSCI.0332-06.2006 (2006).
4 Wan, J. & Goldman, D. Curr Opin Genet Dev 40, 41-47, doi:10.1016/j.gde.2016.05.009 (2016).
5 Thummel, R., et al. Dev Neurobiol 68, 392-408, doi:10.1002/dneu.20596 (2008).
6 Lenkowski, J. R. & Raymond. P. A. Prog Retin Eye Res 40, 94-123, doi:10.1016/j.preteyeres.2013.12.007 (2014).
7 Sherpa, T. et al. Dev Neurobio 68, 166-181, doi:10.1002/dneu.20568 (2008).
8 Reh, T. A., Nagy, T. & Gretton, H. Nature 330, 68-71, doi:10.1038/330068a0 (1987).
9 Coulombre, J. L. & Coulombre, A. J. Dev Biol 12, 79-92 (1965).
10 Karl, M. O. & Reh, T. A. Trends Mol Med 16, 193-202, doi:10.1016/j.molmed.2010.02.003 (2010).
11 Wilken, M. S. & Reh, T. A. Cuff Opin Genet Dev 40, 57-64, doi:10.1016/j.gde.2016.05.028 (2016).
12 Karl, M. O. et al. Proc Na Acad Sci USA 105, 19508-19513, doi:10.1073/pnas.0807453105 (2008).
13 Yao, K. et al. Cell Rep 17, 165-178, doi:10.1016/j.celrep.2016.08.078 (2016).
14 Elsaeidi, F. et al. J Neurosci 38, 2246-2261, doi:10.1523/JNEUROSCI.2126-17.2018 (2018).
15 Ueki, Y. et al. Proc Natl Acad Sci USA 112, 13717-13722, doi:10.1073/pnas.1510595112 (2015).
16 Jorstad, N. L. et al. Nature 548, 103-107, doi:10.1038/nature23283 (2017).
17 Nelson, C. M. et al. J Comp Neurol 520, 4294-4311, doi:10.1002/cne.23213 (2012).
18 Peterson, W. M., et al. J Neurosci 20, 4081-4090 (2000).
19 Ueki, Y., et al. J Neurochem 105, 784-796, doi:10.1111/j.1471-4159.2007.05180.x (2008).
20 Todd, L., et al. Sci Rep 6, 35703, doi:10.1038/srep35703 (2016).
21 Kassen, S. C. et al. Exp Eye Res 88.1051-1064, doi.10.1016/j.exer.2009.01.007 (2009).
22 Zhao, X. F. et al. Cell Rep 9, 272-284, doi:10.1016/j.celrep.2014.08.047 (2014).
23 Erickson, P. A., et al. Invest Ophthalmol Vis Sci 24, 927-942 (1983).
24 Butler, A., et al. Nat Biotechnol 36, 411-420, doi:10.1038/nbt.4096 (2018).
25 Trapnell, C. et al. Nat Biotechnol 32, 381-386, doi:10.1038/nbt.2859 (2014).
26 Qiu, X. et al. Nat Methods 14, 309-315, doi:10.1038/nmeth.4150 (2017).
27 Heinz, S. et al. Mol Cell 38, 576-589, doi:10.1016/j.molcel.2010.05.004 (2010).
28 Castro, D. S. et al. Genes Dev 25, 930-945. doi:10.1101/gad.627811 (2011).
29 Wilken. M. S. et al. Epigenetics Chromatin 8, 8, doi:10.1186/1756-8935-8-8 (2015).
30 McLean, C. Y. et al. Nat Biotechnol 28, 495-501, doi:10.1038/nbt.1630 (2010).
31 Takahashi, K. & Yamanaka, S. Cell126, 663-676, doi:10.1016/j.cell.2006.07.024 (2006).
32 Chronis, C. et al. Cell 168, 442-459 e420, doi:10.1016/j.cell.2016.12.016 (2017).
33 Wapinski, O. L. et al. Cell 155, 621-635, doi:10.1016/j.cell.2013.09.028 (2013).
34 Pollak, J. et al. Development 140, 2619-2631, doi:10.1242/dev.091355 (2013).
35 Benezra, R., et al. Cell 61, 49-59 (1990).
36 Ruzinova, M. B. & Benezra, R. Trends Cell Biol 13, 410-418 (2003).
37 Bai, G. et al. Dev Cell 13, 283-297, doi:10.1016/j.devcel.2007.05.014 (2007).
38 Boareto, M., I et al. Development 144, 3465-3474, doi:10.1242/dev.152520 (2017).
39 Nam, H. S. & Benezra, R. Cell Stem Cell 5, 515-526, doi:10.1016/j.stem.2009.08.017 (2009).
40 Gomez, D. L. et al. PLoS One 10. e0133862, doi.10.1371/joumal.pone.0133862 (2015).
41 Guillemot, F. Prog Neurobiol 83, 37-52, doi:10.1016/j.pneurobio.2007.02.009 (2007).
42 Bonni, A. et al. Science 278, 477-483 (1997).
43 He, F. et al. Nat Neurosci 8, 616-625, doi:10.1038/nnl440 (2005).
44 Barnabe-Heider, F. et al. Neuron 48, 253-265, doi:10.1016/j.neuron.2005.08.037 (2005).
45 Ueki, Y. et al. Glia 60, 1579-1589, doi:10.1002/glia.22377 (2012).

### Example 2: MicroRNAs miR-25 and Let-7 Promote a Neurogenic Potential of Müller Glia in Mice

This Example describes miRNAs from RPCs and MG, and identifies miRNAs more highly expressed in RPCs, and others more highly expressed in MG. To determine whether these miRNAs are relevant to the difference in neurogenic potential between these two cell types, we tested them in dissociated cultures of MG using either mimics or antagomiRs to increase or reduce expression, respectively. Among the miRNAs tested, miR-25 and miR-124 over-expression, or let-7 antagonism, induced Ascl1 expression and conversion of approximately 40% of mature MG into a neuronal/RPC phenotype. These results suggest that the differences in miRNA expression between MG and RPCs contribute to their difference in neurogenic potential, and that manipulations in miRNAs provide a new tool to reprogram MG for retinal regeneration.

In fish and birds, Müller glia (MG) respond to injury of the retina by re-entering the cell cycle and generating retinal progenitor-like cells and ultimately new neurons. In fish, the ability of MG to generate new retinal progenitor cells (RPCs) is in part controlled by miRNAs (for review see (Goldman, 2014). Retinal injury in fish induces the RNA-binding protein Lin28, which functions to reduce levels of the miRNA let-7 and allow expression of Ascl1, critical for regeneration in these species (Ramachandran et al., 2010), for review see (Goldman, 2014). In addition, the downregulation of another miRNA in fish, miR-203, has been reported to increase proliferation of progenitors and is required for retinal regeneration after light damage (Rajaram et al., 2014).

While mammalian retinas also have MG, unlike the fish, MG in the mammalian retina do not naturally generate RPCs in response to injury (Karl et al., 2008; Löffler et al., 2015; Ueki et al., 2015; Wilken and Reh, 2016). The analysis of differences in gene expression between mammalian MG and RPCs (Blackshaw et al., 2004; Brzezinski et al., 2011; Nelson et al., 2011) revealed a set of transcription factor candidates that were subsequently tested in dissociated cell cultures of MG to determine their potential for reprogramming mouse MG to the RPC state. We found that Ascl1 overexpression activated RPC genes and subsequent neuron differentiation in dissociated cultures of mouse MG (Pollak et al., 2013). This same transcription factor (in conjunction with an HDAC inhibitor) was also effective at stimulating functional regeneration of neurons in vivo (Jorstad et al., 2017; Ueki et al., 2015). Similar studies of other candidate reprogramming factors further demonstrated that miRNAs miR-124-9-9* (alone or in combination with Ascl1) (Wohl and Reh, 2016b)) were effective in stimulating the conversion of mouse MG to RPCs and/or neurons. However, a comprehensive survey of miRNAs that differ between progenitors and glia, similar to that carried out for mRNAs, has not been reported.

We used fluorescence activated cell sorting (FACS) to purify RPCs from postnatal day 2 mice and MG from P8, P11, and adult mice. The RNA was extracted from purified RPCs and MG and miRNA expression was analyzed by means of the molecular barcode technology called NanoStrings (Dennis et al., 2015; Geiss et al., 2008). We identified the miRNAs that were more highly expressed in RPCs, as compared with MG, as well as miRNAs that were more highly expressed in MG than RPCs. For the miRNAs that were enriched in the FACS-purified RPCs as compared with the MG, we experimentally over-expressed these in MG cultures to determine if neurogenic competency could be restored. Similarly, for miRNAs that were enriched in the MG relative to the RPCs, we antagonized these in the MG to determine whether this would restore neurogenic competency to the MG. We found that manipulations in two miRNAs, miR-25 (mimic) and let-7 (antagomiR), stimulated neural reprogramming of MG with a neuronal conversion of up to 40% of young MG in vitro. The combination of miR-25 overexpression and let-7 inhibition was even more effective than either treatment alone, with approximately 60% of the Ascl1 expressing MG developing neuronal phenotypes. This reprogramming capacity was decreased in adult MG cultures (range 1-4 months) to approximately 20%. Single cell RNA-Seq of reprogrammed MG confirmed that many of the cells acquired a gene expression profile similar to RPCs and retinal neurons. Together our data show that miRNAs are important in regulating the development of MG and at least one of these, let-7, has a conserved role in the neurogenic competence of both mouse and fish MG.

Results

The miRNA Profile of Retinal Progenitor Cells and Müller Glia in the Mouse Retina

We have previously reported the miRNA expression in Müller glia, using FACS to purify the cells from mature retina (Wohl and Reh, 2016a). To determine which miRNAs are uniquely expressed in RPCs, we used a similar strategy, and FACS-purified RPCs from postnatal day 2 (P2) Sox2-CreER: tdTomatoflSTOP/flSTOP mice. We induced expression of the reporter by tamoxifen injections at P0 and P1 resulting in tdTomato expression in many cells of the neuroblastic layer (NBL). The majority of these cells also expressed progenitor markers Sox9 and Sox2. The fraction of the tdTomato+cells was approximately 50% of the total, somewhat higher than expected. In addition to the RPCs, it is likely that some of the tdTomato+cells were also the neuronal progeny of the RPCs. Moreover Sox2-CreER is also expressed in a small number of amacrine cells. These two Sox2+populations thus reduce the purity of the final sample. To label MG, we used a different strategy that allowed for greater purity of the cells. We FACS-purified the MG at the ages P8, P11 and >P21 from Rlbp1-CreER:tdTomatoflSTOP/flSTOP mice, as previously described (Wohl et al., 2017; Wohl and Reh, 2016a). After tamoxifen application, the majority of MG (Sox9, Sox2, and Glutamine Synthetase (GS)) were labeled; the MG represented 1.5-2.1% of all cells, consistent with previous estimates of MG in the mouse retina (Grosche et al., 2016; Jeon et al., 1998).

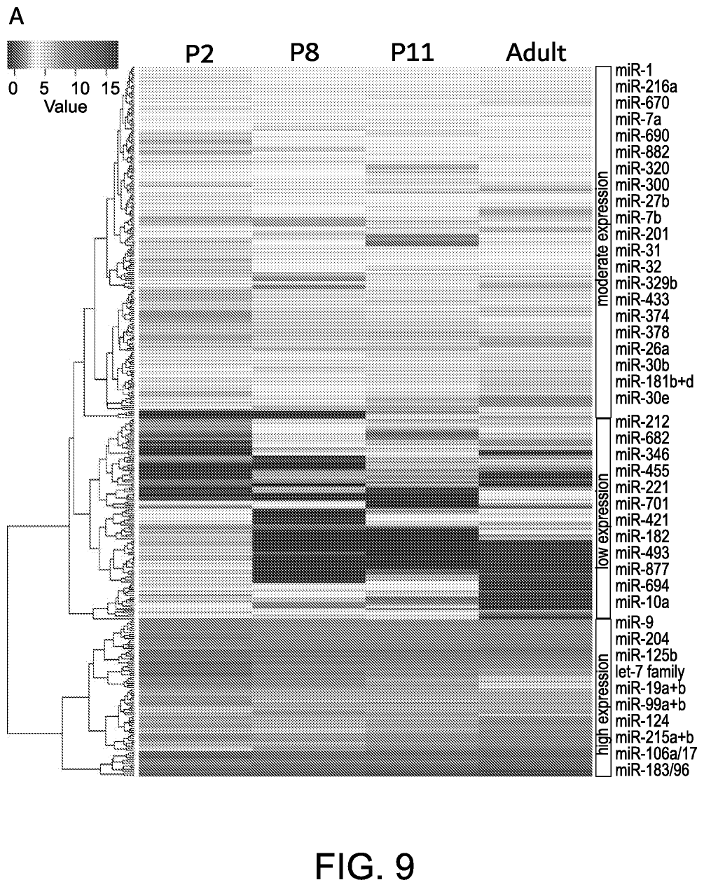

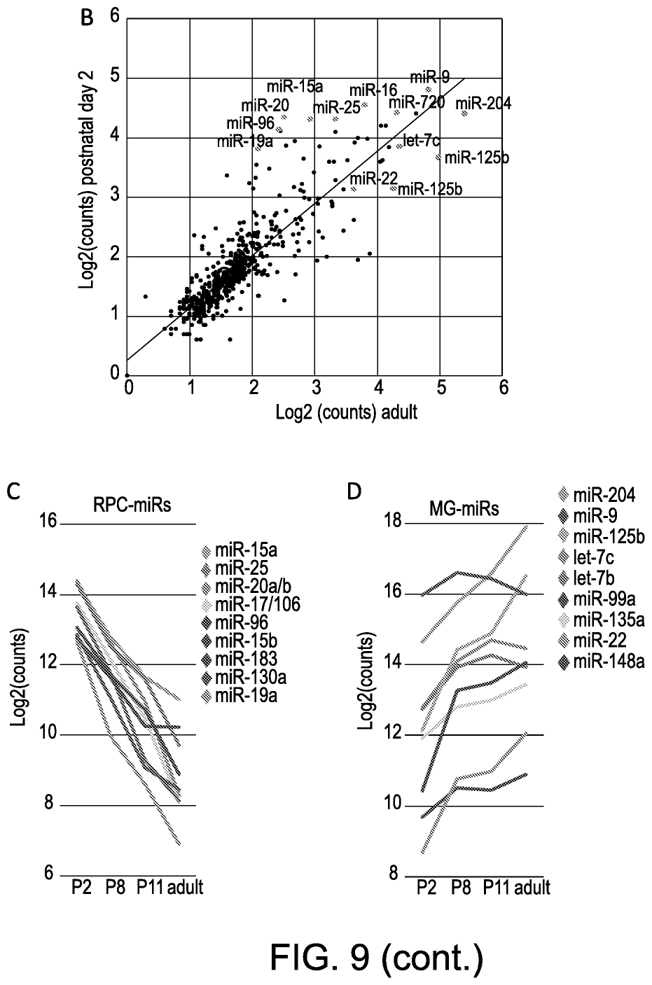

To quantify the miRNAs expressed in RPCs and MG, total RNA was extracted from FACS-purified Sox2:tdTomato+ and Rlbp1:tdTomato+cells. The miRNAs were measured by solution hybridization using the NanoString nCounter® assay (Geiss et al., 2008). The expression profiles of RPCs and MG are shown in FIG. 9A as a heatmap of all ages. We found three main clusters, a green cluster with miRNAs moderately expressed, a blue cluster with miRNAs with low expression, and a pink cluster with the most highly expressed miRNAs. Interestingly, the overall miRNA expression profile of RPCs and MG are quite similar, in accordance with the known similarity in the transcriptome of these cells (Jadhav et al., 2009; Roesch et al., 2008). To better visualize the miRNAs differentially expressed between RPCs and mature MG, we plotted the miRNA expression levels of P2 RPCs and adult MG as a scatterplot, shown in FIG. 9B. miRNAs expressed more highly in RPCs than MG are above the line, while miRNAs expressed more highly in MG than RPCs are below the line. Of 600 miRNAs analyzed, we focused on 11 that were both highly expressed and had substantially lower levels of expression in RPCs than in MG (FIG. 9C). Among the miRNAs more highly expressed in RPCs were members of the miR-17 family i.e., miR-106/miR-17, miR-20a/b, miR-15a/b, miR-19a, and miR-25. These miRNAs are in three clusters in the mouse genome and may be transcribed together. Several of these miRNAs have already been implicated in neurogenesis and cell proliferation in other areas of the nervous system (Beveridge et al., 2009; Foshay and Gallicano, 2009; Jin et al., 2016; Jin et al., 2018; Mao et al., 2014; Naka-Kaneda et al., 2014; Trompeter et al., 2011; Yang et al., 2017). By contrast, the 9 most highly expressed miRNAs that had the greatest increase from P2 RPCs to P8 or older MG, included miR-204, miR-125b, members of the let-7 family (let-7c and let-7b) and the related miRNA, miR-99a (FIG. 9D). let-7 antagomiRs and miR-25 mimics induce Ascl1-reporter expression in MG

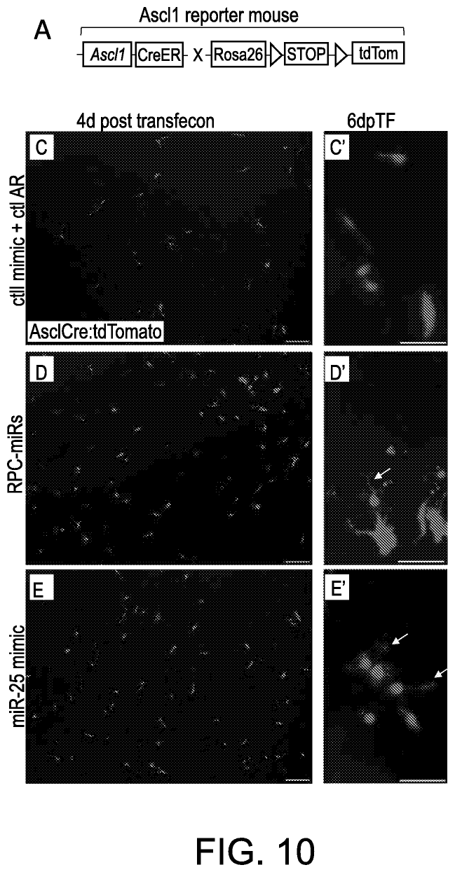

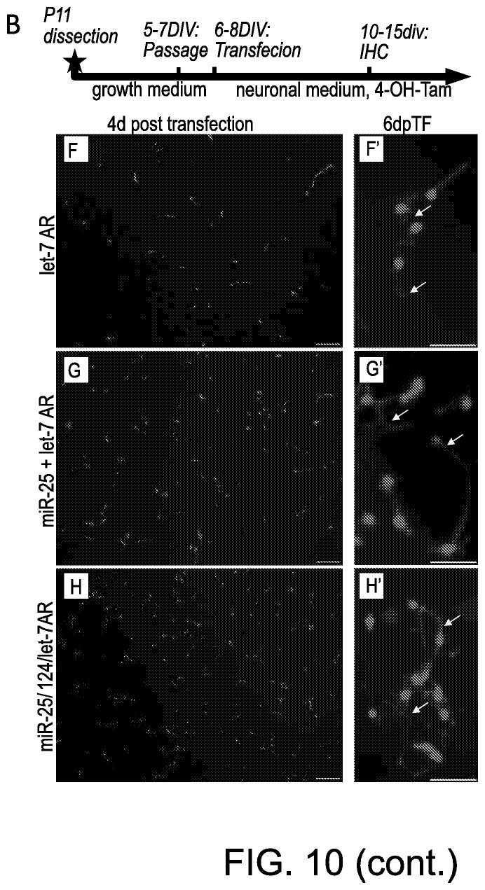

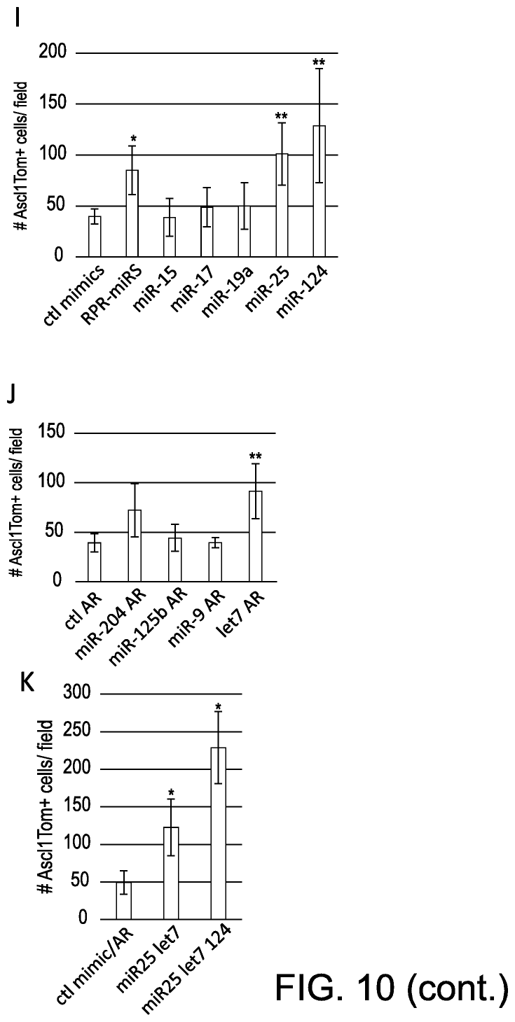

To test whether miRNAs regulate the neurogenic competence of MG, we used primary dissociated cell cultures, since this method has proven effective for identifying reprogramming factors that are effective both in vitro and in vivo (Jorstad et al., 2017; Pollak et al., 2013; Ueki et al., 2015). We hypothesized that miRNAs highly expressed in RPCs may be important in establishing and maintaining the RPC gene expression pattern. One prediction of this hypothesis is that over-expression of the RPC-miRNAs in MG would cause the MG to adopt a progenitor-like gene expression profile. To test this prediction, we transfected dissociated cultures of MG with miRNA mimics (double-stranded, preprocessed miRNAs). For the initial screen we used MG cultured from Ascl1-CreER:tdTomatofSTOP/flSTOP reporter mice, to label cells that express the progenitor gene Ascl1 (FIG. 10A). Out of the most highly expressed RPC miRNAs (i.e., high in RPCs but low in MG), we used one candidate from every family (i.e., containing the same seed sequence). Specifically, we tested mimics for miR-15a, miR-17, miR-19a, and miR-25. We included miR-124 as a positive control, since our previous report demonstrated that this miRNA also induces Ascl1 expression and neuronal gene expression in MG (Wohl and Reh, 2016b). Two other miRNAs that were more highly expressed in the RPC sample than the MG samples were miR-183 and miR-96. These have been previously localized to photoreceptors (Busskamp et al., 2014; Xiang et al., 2017; Xu et al., 2007; Zhu et al., 2011), and possibly represent a contaminating population of photoreceptors in the P2 RPC population. These were not tested in this assay.

MG cultures from P11 Ascl1-CreER:tdTomatoflSTOP/flSTOP mice were transfected with either a cocktail of all four RPC-miR mimics, or each miRNA mimic individually (FIG. 10B). We added 4-Hydroxy-tamoxifen to the cultures to activate the cre-recombinase and monitored the cells for tdTomato expression. After four days post-transfection, we found a few cells expressing the reporter in control MG (FIG. 10C). By contrast, the cocktail of all four RPC-miRNAs (FIG. 10D), miR-25 alone (FIG. 10E), as well as miR-124 led to a significant increase in Ascl1:tdTomato+cells (FIG. 101). miR-15a, miR-17, and miR-19a however, did not cause an increase in the number of reporter positive cells. After 6-8 days post transfection, some Ascl1:tdTomato+cells transfected with either the RPC-cocktail or miR-25 alone underwent morphological changes and took on a more neuronal-like morphology, with small somata and long processes (FIG. 10C‘-E’). Since miR-25 alone had a similar effect to the RPC-cocktail, we used miR-25 alone in additional experiments.

Next, we focused on the miRNAs that increase with glial maturation. The miRNAs expressed more highly in MG than RPCs included the previously described mGliomiRs miR-204, miR-125, miR-9, miR-99a, and miR-135a, as well as miR-22, miR-148a, let-7a and let-7c. We hypothesized that the increase in these miRNAs in MG might prevent them from expressing progenitor genes. One prediction of this hypothesis is that antagonism of these miRNAs would promote RPC gene expression and neuron differentiation. To test this prediction, we used complementary hairpin inhibitors called antagomiRs (AR). We tested these miRNA ARs either alone or in combinations. Interestingly, the only ARs that induced the Ascl1-reporter were miR-204AR and let-7AR (either let-7a or let-7c, FIGS. 10F,J), and only after let-7 inhibition, did we observe cells with a neuronal morphology (˜40%, FIGS. 10F′), similar to the effect of miR-25 mimic overexpression.

We then combined both approaches: cells were transfected with miR-25 mimic and let-7AR, with or without miR-124, in MG cultures from the Ascl1 reporter mice to determine whether the combination was more effective than either manipulation alone (FIGS. 10G,H). The combination of miR-25, let-7AR, and miR-124 led to a further increase of Ascl1 expression in the MG (FIGS. 10G,H,K). In addition, in the controls (control mimics and control antagomiRs combined) Ascl1:tdTomato+displayed a flat cell morphology, whereas Ascl1:tdTomato cells treated with miR-25+let-7AR or miR-25+let-7AR+miR-124 mimics adopted a neuronal morphology, with a small somata and various fine processes (FIG. 10G′,H′). In all of the combinations, there were more Ascl1:tdTomato+cells and a larger fraction of neuronal-like cells (50-60%) compared to in the single treatments.

Let-7 Antagomirs and miR-25 Mimics Reprogram MG to Neural Progenitors and Neurons

In order to confirm the neuronal identity of the cells in the miR-treated conditions, Map2 and Otx2 immunofluorescent labeling and confocal microscopy were carried out (FIG. 11). Many of the Ascl1:tdTomato+neuronal-like cells were positive for Map2 and they comprised 52-54% of all Ascl1:tdTomato+cells (FIGS. 11C-G). These neuronal cells were either highly branched or had one long process with a growth cone-like structure. (FIG. 11F arrowhead and arrows respectively). Approximately 44% of the miR-25/let-7AR transfected cells expressed Otx2, with close to 30% expressing both Map2 and Otx2. Slightly higher percentages of neuronal-like cells were obtained in the miR-25/miR-124/let-7AR transfected cells, with almost 60% of the Ascl1:tdTomato+cells expressing Otx2 and 40% being positive for both Map2 and Otx2 (FIGS. 11H,1). Although the percentages of Map2+(35-62%), Otx2+(20-38%), and Map2+/Otx2+cells (10-20%) in the single treatments were also high, the overall number of neurons was lower than in the cocktail treated wells. These results extend our previous observations with miR-124 and show that the combination of miR-25 overexpression and let-7 inhibition, with or without miR-124 overexpression, leads to a significant increase in Ascl1 expressing MG in vitro, with approximately half of the Ascl1:tdTomato+expressing Map2+ and/or Otx2+.

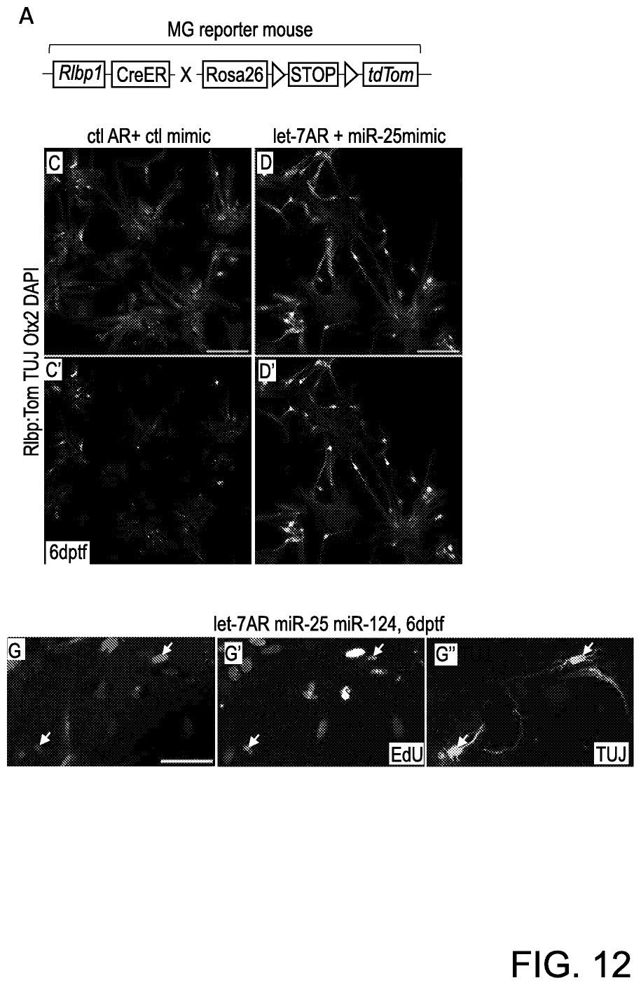

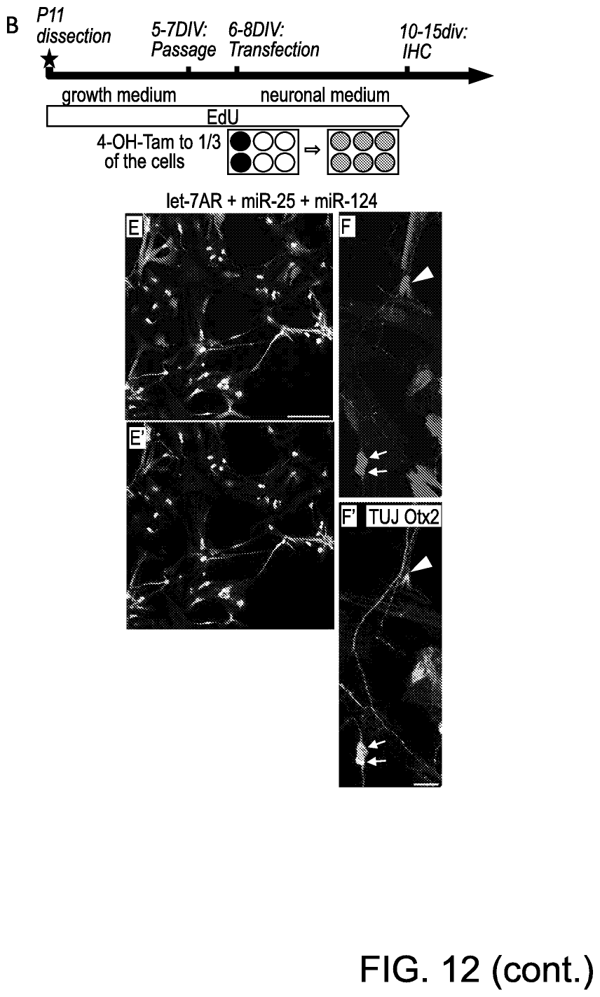

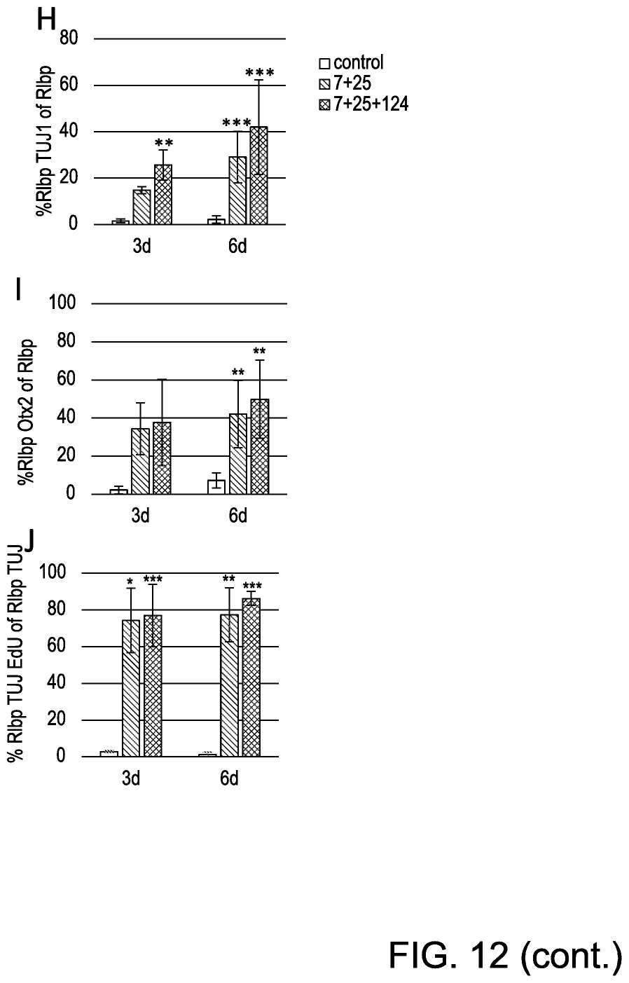

The experiments described above were carried out with MG that express the Ascl1 reporter. Since only a relatively small subset of MG express this reporter, even in the treatment conditions, we also used an additional MG reporter (Rlbp1-CreER: tdTomatoflSTOP/flSTOP) that is expressed in all MG for the next series of experiments (FIG. 12A). In this line of mice, all MG express the reporter, so visualization of individual cells in the cultures is difficult. Therefore, we used the following strategy to label only a third of the MG: P11 retinas from the Rlbp1-CreER:tdTomato mice were dissociated and plated as usual, but 4-hydroxytamoxifen was added only to 2 out of 6 wells to induce reporter transgene expression. When the cells were passaged, the tdTomato expressing cells were combined with the untreated cells, such that approximately one third of the cells in each well expressed the reporter (FIG. 12B). These cultures were then transfected with the miR-25/let-7AR, miR-25/miR-124/let-7AR or control mimics/ARs.

The results for the Rlbp1-CreER:tdTomato MG were similar to those we obtained with the Ascl1:tdTomato cultures: treatment with miR-25 with let7-AR with or without miR-124 caused the Rlbp1-tdTomato+cells to adopt a neuronal morphology, with small somata and fine branched processes (FIGS. 12C/C′-G-G″). Immunofluorescent labeling confirmed that the cells expressed neuronal genes, with approximately 30% of the miR-25/let-7AR treated cells and 40% of the miR-25/let-7AR/miR-124 treated cells expressing TUJ1 (control: 2%, FIG. 12H). In addition, approximately 40% of the miR-25/let-7AR and 50% of the miR-25/let-7AR/miR-124 treated MG expressed Otx2 six days post transfection, while only 7% of the cells in control wells expressed this marker (FIGS. 12F/F′,I). To further confirm that the neuronal cells arose from proliferating MG, we added EdU at the onset of the MG cultures (FIG. 12B), which results in labeling of all dividing MG. About 80% of all Rlbp1+TUJ+were also EdU+showing that these neurons originated from MG that underwent mitotic proliferation in vitro (FIG. 12G-G″, J). Note that the presence of TUJ1+EdU+Rlbp1cells (FIG. 12G-G″ arrow) is likely due to the fact that only one third of the MG were treated with 4-hydroxytamoxifen and hence labeled with the reporter.

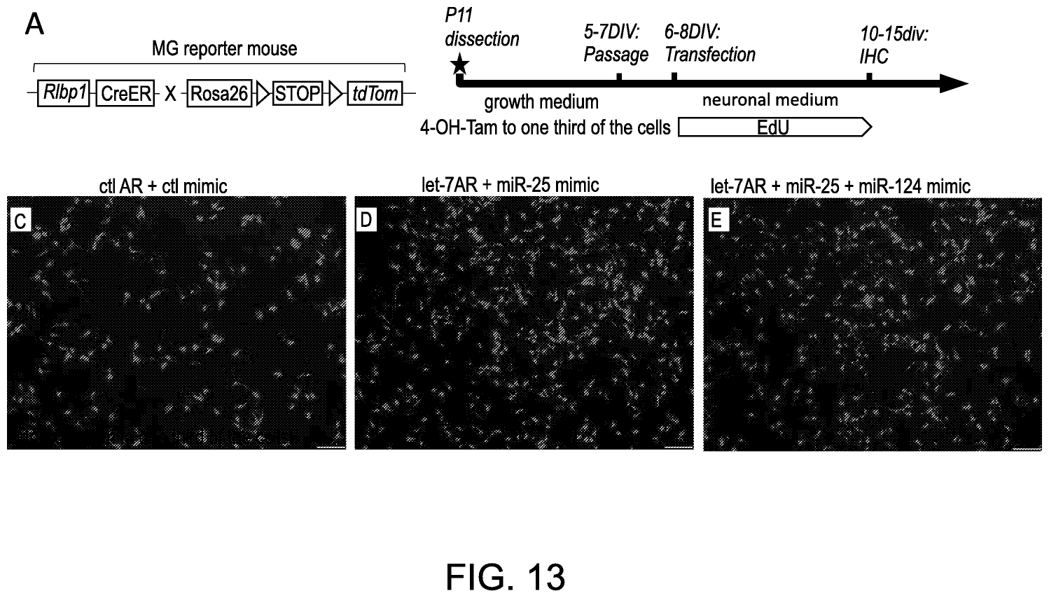

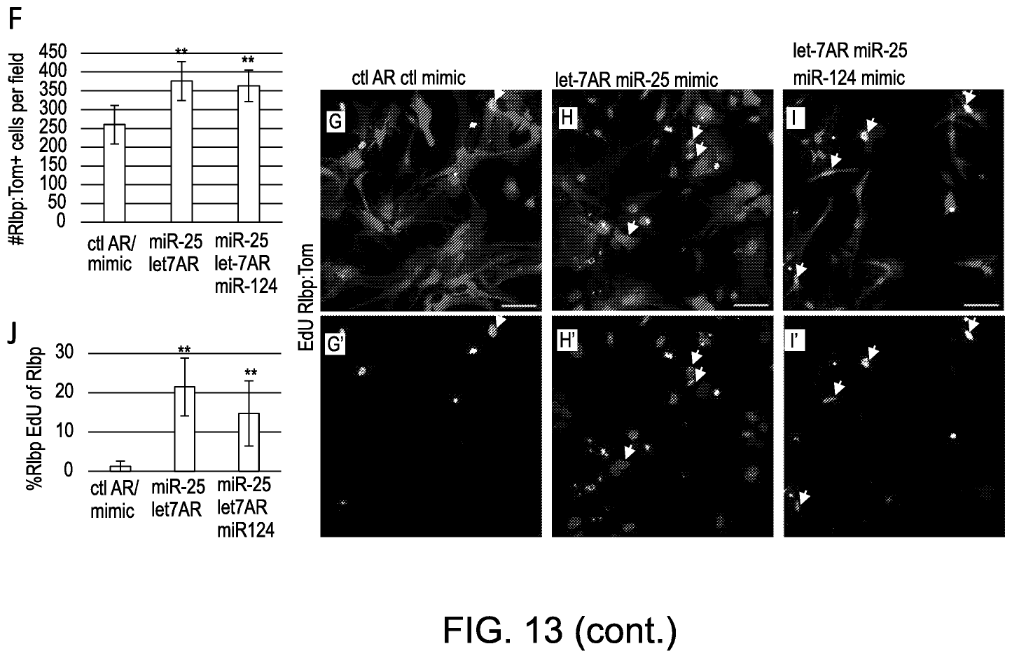

Taken together, these data indicate that miRNAs impact the ability of MG to generate neurons in vitro. To determine whether these treatments also affect MG proliferation, we added EdU after transfection (FIG. 13) to label S-phase. We compared the cell numbers in the treated and control cultures. Six days after transfection we found a significant increase in Rlbp1:tdTomato+cells for both cocktails (FIG. 13C-F), relative to controls suggesting that the treatments increased the proliferation of the MG. EdU labeling confirmed that approximately 20% (miR-25/let-7AR) and 15% (miR-25/let-7AR/miR-124) of all Rlbp1:tdTomato had incorporated EdU, a significant increase over the control wells (FIG. 13G-J).

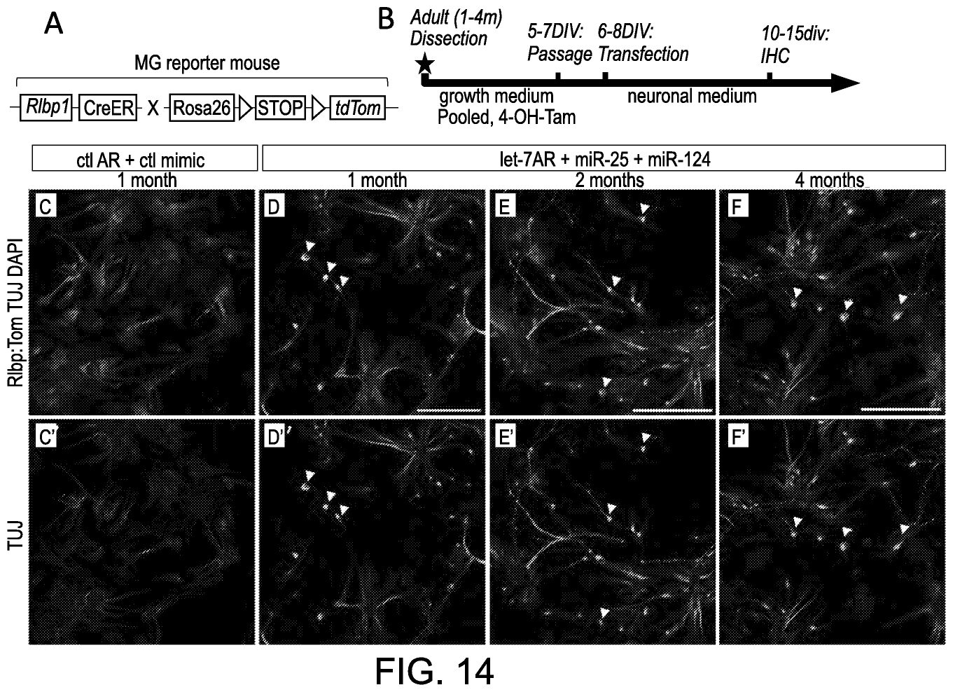

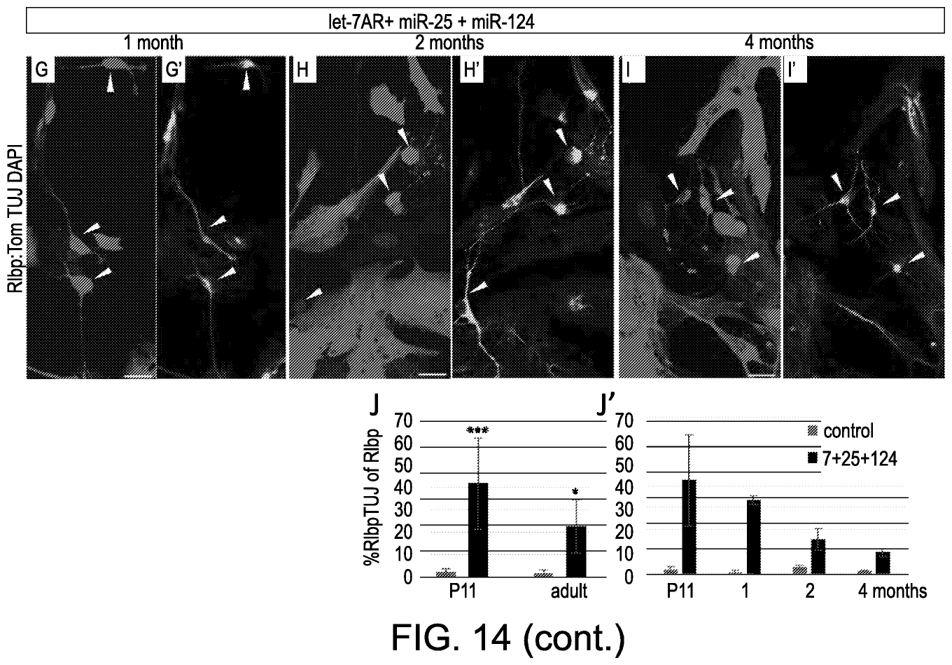

The experiments described above used MG isolated from the P11 mouse retinas. At this age, there are no progenitors remaining in the retina, but the MG still proliferate robustly in dissociated cell cultures (Pollak et al, 2012). The MG of older mice can be grown in vitro by using a feeder layer of young MG (Wohl et al., 2017) or if the adult MG are plated at high density (this study; FIG. 14). Using the latter method, we cultured MG of mice from one to four months of age and transfected them with the two miRNA cocktails (FIGS. 14A,B). The MG from the mature mice responded to the miRNA cocktails much like the P11 cultures: there were Rlbp1:tdTomato+cells that had small somata, fine processes and were TUJ1+(FIGS. 14C/C′-F/F′ arrowheads). These were present as single, isolated cells or in clusters of 3 to 6 cells, often associated with a flat MG (FIGS. 14G/G′-I/I′). The potential of MG to convert into neurons declines with age: in the P11 MG cultures, approximately 40% differentiate into neuron-like cells, while in the adult MG cultures (range 1-4 months) an average of 20% of the cells express TUJ1 after treatment with the miRNA reprogramming cocktail (FIG. 14J/J′).

Single Cell RNA-Seq Analysis of Reprogrammed MG

To better characterize the mechanisms involved in the effects of miR-25 mimics and let-7 antagonism on MG fate, we carried out single cell RNA-seq (scRNA-seq). Cultured MG that had been transfected with either control mimics/ARs, or one of four experimental groups (let-7AR, miR-25 mimic, miR-124 mimic, miR-25 and let-7AR or all three combined) were processed for single cell analysis on the 10× Genomics platform. The cells were harvested 8 days after transfection and processed for single cell sequencing. After running the reads through the CellRanger pipeline, data was analyzed and normalized using the Seurat package. Linear dimensionality reduction was carried out using a principal component analysis (PCA), cells were clustered based on PCs, and clusters visualized on tSNE plots.

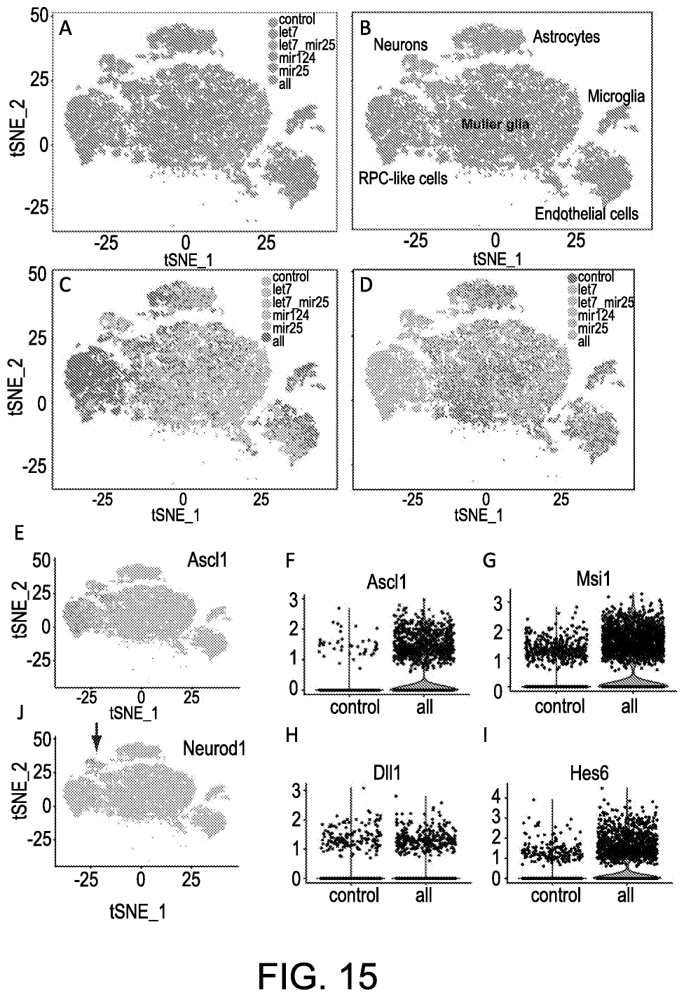

In all wells, the cells were primarily MG, but other contaminating cell populations (e.g. microglia, endothelial cells, astrocytes) were also identified by their gene expression profiles (FIGS. 15A,B). Most fibroblast-like cells, were removed from further analysis, and the MG cells from control and treated cultures were combined to better characterize differences among the treatment conditions. In both control and treated conditions, most of the MG had a similar pattern of gene expression, though their differences allowed them to form several clusters. One cluster in particular, had a retinal progenitor cell (RPC)-like gene expression profile and contained cells that expressed Ascl1 and its downstream targets, like Hes6, Msi1 and DII1. This cluster was primarily formed by cells that were treated with the combination of let-7AR with miR-25 and miR-124 mimics (“all”, FIGS. 15C-1). The induction of Ascl1 and other progenitor genes in a subset of the MG by miR-25 mimics and let-7AR confirms the results with the Ascl1 reporter (FIG. 10). This result further suggests that the decline in Ascl1 during development, that accompanies the transition from RPCs to MG, is in part regulated by miRNAs. In addition to the progenitor-like cells, a smaller group of cells expressed more mature markers of retinal neuronal differentiation, including Neurod1 (FIG. 15J), Doublecortin, Cabp5, Otx2, Neurod4, Grm6, and Gap43 (growth cones see FIG. 11F arrows) as well as synaptic markers such as Synaptophysin and Snap25. Results from the scRNA-seq analysis extend the data from the immunofluorescence analyses and further support the idea that miRNAs can be used to reprogram MG to RPCs and neurons, similar to the over-expression of Ascl1.

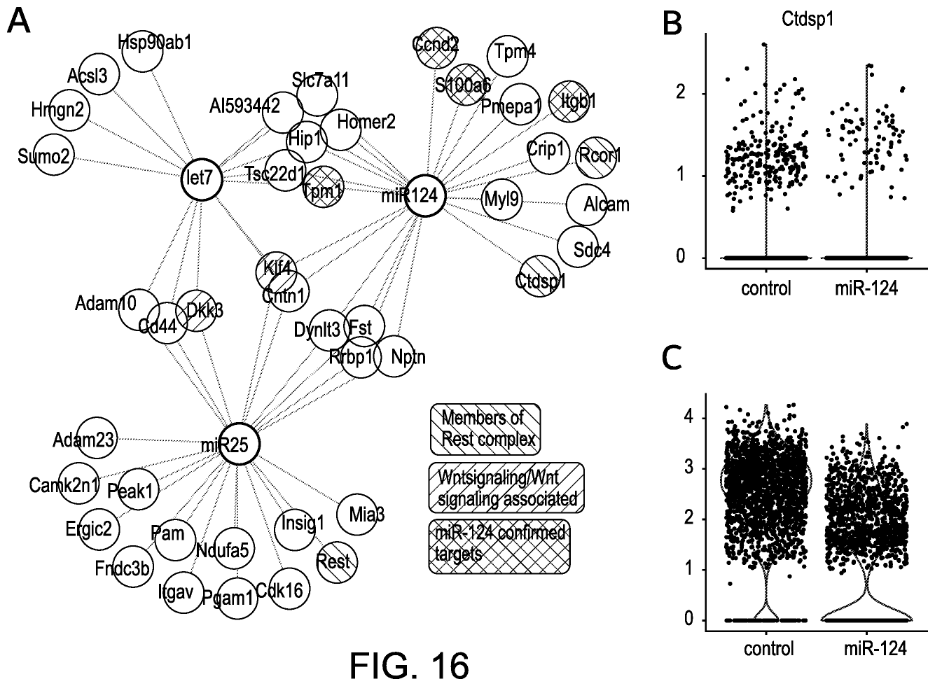

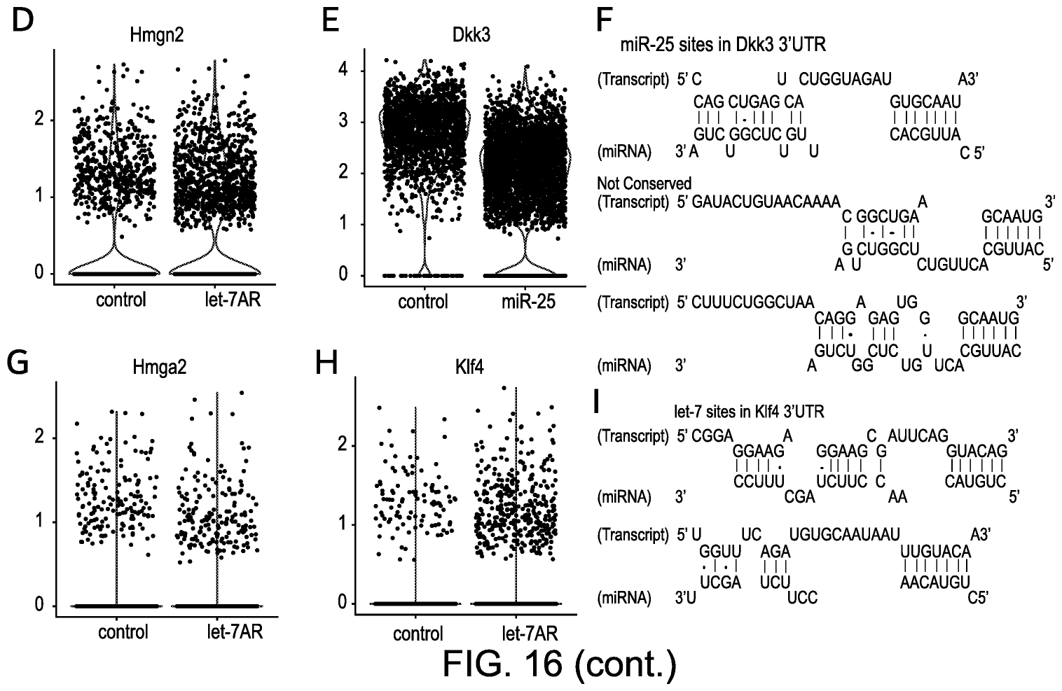

To identify potential targets of miR-25, miR-124 and let-7 in MG, we compared each single treatment to the control cells using Seurat. We selected only the clusters that contained the MG, based on their expression of MG-specific genes. We then used “Find Markers” in Seurat to select those genes that were most highly differentially expressed between MG in the treatment and the control conditions. We reasoned that those genes targeted by miR-25 or miR-124 would be reduced in the cells treated with these mimics, while the genes targeted by let-7 should show increases in the scRNA-seq data in the antagomiR treated cells. For the cells treated with mimics for either miR-124 or miR-25, we selected those genes (top 100) with the greatest fold decreases, while for the let-7 antagomiR treated cells, we selected those genes that showed the greatest fold increase. These genes were then analyzed using the multimiR package in Bioconductor to identify miRNA targets. The potential targets are shown as a graph, with the genes and miRNAs as the nodes in FIG. 16A.

Many of the potential targets of let-7, miR-25 and miR-124 are unique to each of these miRNAs. However, some of the genes that change the most in the scRNA-seq, like transcription factor Klf4 (Kruppel-like factor 4) and the Wht antagonist Dkk3 (Dickkopf 3), are targeted by two or three of these miRNAs (pink dots in FIG. 16A). Thus, some of the synergy observed in our experiments may stem from several miRNAs affecting the same target. Another key point is that targets of miR-124 that were identified in our analysis, Tpm1 and Itgb1, (FIG. 16A) have been previously identified as miR-124 targets in other cells (Hunt et al., 2011; Idichi et al., 2018; Neo et al., 2014). One gene in particular, Ctdsp1 (FIG. 16B), a member of the Rest complex which has been reported before to be a target of miR-124 (Nesti et al., 2014; Visvanathan et al., 2007), was identified in our earlier study of MG reprogramming with miR-124, and regulates Ascl1 via the Rest pathway (Wohl and Reh, 2016b). It is therefore interesting that another of the top targets identified in this analysis is Rcor1, since this is also a member of the Rest complex that represses neural genes (Abrajano et al., 2009; Andres et al., 1999; Masserdotti et al., 2015; Qureshi et al., 2010). Some of the synergy between miR-25 and miR-124 in reprogramming Müller glia to an RPC-like state may be due to Rest being potentially targeted by miR-25. Another target of miR-124, the cell cycle gene Cyclin D2 (Ccnd2) (Li et al., 2017), showed a decline in miR-124 treated MG as well (FIGS. 16A, violin plot in C).

The top gene targeted by let-7 (i.e., fold increase after let-7AR) was the transcription factor Klf4 (FIG. 16E). Klf4 is known to be expressed in MG and increases after injury in chick and fish retina (Todd and Fischer, 2015; Todd et al., 2018; Zelinka et al., 2016). Klf4 was among the first genes shown to promote reprogramming of fibroblasts to iPSCs (Gao et al., 2016; Hjelm et al., 2011; Wemig et al., 2008). Although this had not been previously shown to be a target of let-7, there are two predicted, conserved let-7 sites in the 3′UTR of Klf4 (FIG. 16F). Moreover, a recent study reported that let-7 inhibits reprogramming of human cells into iPSC using the known reprogramming factors including Klf4 (Worringer et al., 2014). Another gene that is expressed in MG and is known to be a target of let-7 in many cell types is The High motility group A2 protein (Hmga2) (Balzeau et al., 2017; Lee and Dutta, 2007; Patterson et al., 2014; Xia and Ahmad, 2016). There was a decline in the Hmg family member, Hgmn2 (FIG. 16D), but not in Hmga2 in the let-7AR treated MG (FIG. 16G)

Other potential targets identified in our analysis have not been previously reported to be targets of these miRNAs. In terms of fold-change, the top target of miR-25 was Dkk3, a Wht inhibitor (FIG. 16H). Since activation of the Wht pathway has previously been shown to stimulate MG proliferation (Das et al., 2006; Gallina et al., 2016; Nakamura et al., 2007; Ramachandran et al., 2011; Yao et al., 2016), the effect on Dkk3 might in part explain the increase in MG proliferation we observe (FIG. 13). Moreover an increase in Wnt signaling leads to activation of Wnt target genes such as the neuronal gene NeuroD1 (see list at www.stanford.edu), which was found in the scRNA-seq analysis (FIG. 15J). There are three predicted sites in the Dkk3 3′UTR (FIG. 161) and two studies which report Dkk3 is a direct target of miR-25 for other cell types (Huo et al., 2016; Yoshida et al., 2018) and two studies reporting that the miR-92 targets Dkk3 in neural tissue (De Brouwer et al., 2012; Haug et al., 2011). miR-92 has the same seed sequence as miR-25.

Discussion

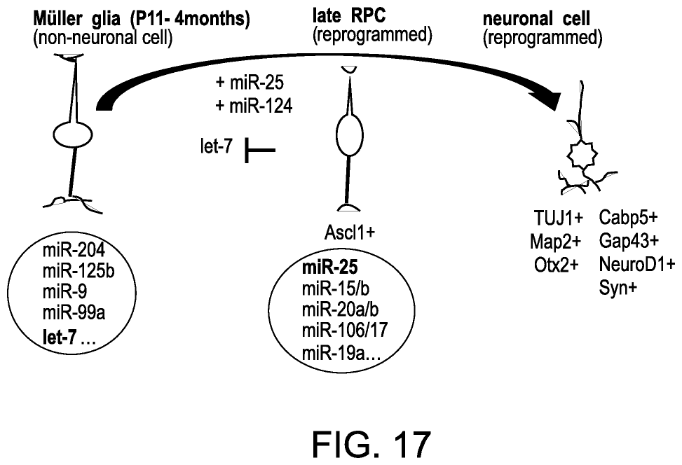

This Example provides an analysis in the differences in miRNA expression between RPCs and MG. The differences in miRNA expression coincide with differences in mRNA expression between the two cell types, and reflect the relationships among these classes of molecules during the cell fate transition from RPCs to MG. One of the most important differences between RPCs and MG is their capacity for mitotic proliferation. RPCs are highly proliferative cells, while MG are quiescent in mice. Cell cycle genes are thus highly expressed in RPCs, but down-regulated in the MG (Nelson et al., 2011). Coincidently, many miRNAs known to be important in cell cycle gene regulation are highly expressed in RPCs, but not in MG. Examples include miR-15a/b, miR-19a, miR-17, miR-106, and miR-20a/b (summarized in FIG. 17). These are among the miRNAs that show the largest differences between the MG and RPCs and decline as the MG mature, and are well-established regulators of cyclins and cyclin-dependent kinases (Bueno and Malumbres, 2011). Some of the most highly expressed set of miRNAs, in RPCs, those from the miR-106b-25 cluster (miR-106b, miR-92, miR-25), are products of the intron in the DNA replication licensing factor Mcm7, and so the levels of these miRNAs in RPCs might also be due to the much higher level of expression of Mcm7 in RPCs than MG.

In addition to the changes in mitotic cell cycle that occur as RPCs differentiate into MG, there are also changes in the competence of the latter to generate neurons. Proneural genes, such as Ascl1 and Neurog2 are rapidly down-regulated in the MG, along with other genes associated with neurogenesis, like Musashi (Msi1) and Nestin. Previous studies in other areas of the nervous system have shown that miRNAs are critical regulators of neurogenesis and neurodevelopmental patterning. For example, miR-106 and miR-17 are necessary for the neurogenic to gliogenic transition in neural stem/progenitor cells from cerebral cortex: over-expression of miR-17 inhibits the acquisition of gliogenic competence by targeting p38/Mapk14 (Naka-Kaneda et al., 2014).

To better understand the roles of miRNAs in the processes of cell proliferation and neurogenesis in RPCs and MG, we manipulated the expression of those that exhibited the greatest differences between these cells. Of the miRNAs we tested, we found the most significant effects with miR-25 and miR-124 mimics, and let-7 antagomiRs. As noted above, miR-25 is part of a cluster with miR-92 and miR-106b, in an intron in the Mcm7 gene. A previous report demonstrated the importance of the miR-106b-25 cluster in primary cultures of neural stem progenitor cells, where knocking down miR-25 reduced neural stem/progenitor cell proliferation, whereas over-expression of the cluster increased neuron production (Brett et al., 2011). We find that miR-25 has similar effects on MG: increasing the levels of this miRNA with a specific mimic, causes an increase in Ascl1+MG derived neuronal cells. Thus, miR-25 may have a more general role in maintaining the neural progenitor phenotype throughout the nervous system.

Another miRNA that was active in our reprogramming assay was let-7, a miRNA that has been already shown to be important for both neurogenesis and neural regeneration in previous reports. Originally identified as part of the heterochronic pathway in C. elegans, let-7 has important developmental roles in many tissues and organisms (Pasquinelli et al., 2000; Reinhart et al., 2000). The production of mature let-7 is regulated by Lin-28, another component of the heterochronic pathway in worms (Johnson et al., 2003; Reinhart et al., 2000) and one of the first genes identified as a reprogramming factor for generating iPSCs (Yu et al., 2007). In the embryonic mouse retina, let-7 and Lin-28 also have a critical role in regulating developmental timing, specifically the transition of the RPC from generating early fates to generating late cell identities (La Torre et al., 2013). A key step in retinal regeneration in zebrafish is the induction of Lin-28 by Ascl1. In this system, Lin-28 also inhibits let-7 maturation and this causes a further increase in Ascl1 (Goldman, 2014; Ramachandran et al., 2010). Although we have not observed that Ascl1 over-expression in MG can induce Lin-28 in mice, the second part of the feedback loop may be intact in mice, since let-7 inhibition leads to an Ascl1 increase in mice (this study). Moreover, over-expression of Lin-28 in fish MG has been shown to stimulate their proliferation and expression of neural progenitor markers (Elsaeidi et al., 2018; Ramachandran et al., 2010; Yao et al., 2016). In mice, Yao et al reported that MG proliferation and their neurogenic potential is regulated via Wnt signaling through a Lin-28/let-7 dependent pathway (Yao et al., 2016).

The third miRNA that we find reprograms MG to a more RPC like state is miR-124. We previously reported that miR-124 could reprogram MG, by targeting genes in the Rest pathway, including Ctdsp1 and Ptbp1 (Wohl and Reh, 2016b). In this study, we confirmed these findings with single cell RNA-seq, demonstrating that miR-124 causes increases in neural genes and neural progenitor genes, and a decrease in the expression of Ctdsp1. The relationship between miR-124 and the Rest pathway has been previously investigated in neural reprogramming in fibroblasts using miR-124 in combination with miR-9/9* that target different members of the Rest complex (Abemathy et al., 2017; Victor et al., 2014; Xue et al., 2013; Yoo et al., 2009; Yoo et al., 2011). One of the mechanisms important for the miRNA-mediated conversion of fibroblasts to neurons involves the gene Ptbp1, an inhibitor of miR-124 (Makeyev et al., 2007; Xue et al., 2013). Recent evidence shows that the mechanism of miR-124/9/9* also involves Usp14, Ezh2 and Rest (Lee et al., 2018). The level of Rest is regulated by a stabilizing methylation via the methyltransferase Ezh2, and the level of Ezh2 is in turn regulated by the de-ubiquitinating enzyme, Usp14 (Doeppner et al., 2013; Lee et al., 2018). The miRNAs, miR-124 and miR-9, target Usp14, which leads to a decrease in Ezh2, a loss of methylation on Rest, and increased degradation of this neural gene repressor (Lee et al., 2018). The finding that ablation of Rest dramatically improves the efficiency of neural reprogramming in astrocytes (Masserdotti et al., 2015) indicates that some of these same mechanisms may be active in repressing neural genes in glia. In the MG, Usp14 was not one of the genes that changed significantly in the miR-124 treated cells, but Ezh2 deletion is known to have effects on retinal development (Zhang et al., 2015), including an early onset of neuron differentiation, consistent with a similar role in RPCs.

An interesting result from our study suggests miRNA regulation of Ascl1 levels in MG and possibly RPCs. There is not a lot known about the regulation of Ascl1 expression or levels in RPCs. Our results in this paper and our previous report of miR-124 in MG reprogramming suggest that the Rest pathway is involved, either directly or indirectly, in the regulation of this key transcription factor. Since our initial screen relied on changes in an Ascl1 reporter, it is not surprising that we find the miRNAs effective in this screen were those that induce increases in Ascl1 in the single cell RNA-seq, though the 3′UTR of Ascl1 does not appear to be targeted directly by these miRNAs. It is possible that miR-25 and let-7 also regulate Ascl1 levels via the Rest pathway, and there is a predicted site in miR-25 for Rest. However, it is also possible that other genes targeted by these miRNAs, such as Dkk3 and Klf4, regulate Ascl1 expression. It is also worth noting that we only screened a subset of the most highly differentially expressed miRNAs in our assay, and it is likely that additional miRNAs are important in the maintenance of cell state in the RPCs and MG; nevertheless, our results show that antagonizing let-7 and increasing levels of miR-25 and miR-124 with miRNA mimics, is useful in reprogramming MG to retinal neurons in vitro and could help in stimulating regeneration in this system.

Materials and Methods

Animals

All mice were housed at the University of Washington and all experiments were carried out in accordance with University of Washington Institutional Animal Care and Use Committee approved protocols (UW-IACUC). Sox2-CreERT2 (017593) mice were obtained from Jackson laboratories, Ascl1-CreERT2 (012882) mice were a gift of Dr. Jane Johnson (UT Southwestern Medical School), and Rlbp1-CreERT2 mice were obtained from Dr. E. Levine at Vanderbilt University (Vazquez-Chona et al., 2009) All cre lines were crossed to R26-stop-flox-CAG-tdTomato mice (Jackson Laboratories, also known as Ai14 [007914]). Tamoxifen (Sigma) was administered intraperitoneally at 75 mg/kg in corn oil at P0+1, P6+7, P9+10 for the P2, P8, and P11 analysis respectively and for two consecutive days at ages P>21 for the adult assay. Males and females were used. Strains and ages are specified in every Figure.

Fluorescence Activated Cell Sorting (FACS)

Retinas of four P2, twenty-six P8, sixteen P11, and 20 adult (>P21) mice were dissociated and confirmed for successful recombination under the fluorescence microscope. For one sort, 6-10 retinas were pooled and dissociated in DNase/Papain (75 μl/750 μl respectively, Worthington) for 20 min at 37° C. on the shaker, triturated, mixed with Ovomucoid (750 μl) to stop the enzymatic reaction, centrifuged for 10 min at 300×g and resuspended in 600 μl DNase/Ovomucoid/Neurobasal solution (1: 1: 10 respectively, Gibco) per retina. Cells were filtered through a 35 μm filter to remove cell clumps, sorted using an 80 micron nozzle, and collected into two separate chilled tubes. Debris was excluded from the sort and only events in gate P1 were sorted. Cells with the brightest fluorescence were found in gate P3 (“positives” (+), the RPC or MG fraction), while cells with no fluorescence were in gate P2 (“negatives” (−)), and everything in between was excluded. Post-sorts of the tdTomato+RPC/MG were performed to assure high purification (a 85% tdTomato+cells of total cells), which was validated as described before (Wohl and Reh, 2016a). Samples were collected in bovine serum albumin (BSA) coated tubes containing Neurobasal medium. Cell sorts were performed using of BD Aria III cell sorter (BD Bioscience). After collection, the tdTomato+MG fractions were post-sorted to validate purity. In addition, one drop of each condition was plated on a coverslip and evaluated for purity. All other cells were spun for 10 min at 300×g at 4° C., the pellet was homogenized in Qiazol (Qiagen) and stored at −80° C.

Müller Glia Primary Culture

Müller glia were dissociated (see above) from postnatal day (P) 11/12 mice and adult (one month, 2, and 4 months) and grown in Neurobasal medium supplemented with N-2, tetracycline-free 10% fetal bovine serum (FBS, Clontech), and epidermal growth factor (EGF, R&D Systems, 100 ng/mL) as described previously (Ueki et al., 2012). After 5-7 days in vitro (DIV), cells were either passaged on 6 well or Poly-Omithine (Poly-O, Sigma) and Laminin (Gibco, Life Science Corporation) covered coverslips in 24-well plates. EdU was added either with the onset of culturing to track cell proliferation of MG lineage or after transfection to quantify cell proliferation due to the treatment.

Transfection

miRNA mimics and/or antagomiRs (Table S4, Thermo Scientific; 500 nM each) were used for RNA transfection. MG cultures from Ascl1-1CreER:tdTomato or Rlbp1-CreER:tdTomato mice were transfected using Lipofectamine 3000 in Optimem medium, in accordance with manufacturer's instructions. Three hours after transfection, the media was changed to either normal medium (1% FBS with B27 supplement and BDNF (100 ng/mL)) or BrainPhys neuronal medium (Stemcell Technologies) supplemented with B27, N2, BDNF (20 ng/mL), GDNF (20 μg/mL), Dibutyryl-cAMP (1 mM), and ascorbic acid (200 nM, see (Bardy et al., 2015).

RNA Purification and miRNA Profiling

The sorts of all retinas per age were pooled for the RNA-purification. RNA was extracted and purified with a miRNeasy Micro Kit in accordance with manufacturer's instructions (Qiagen). NanoString nCounter® software was used for miRNA expression analysis. DNA sequences called miRtags were ligated to the mature miRNAs through complementary oligonucleotides with sequence-specific binding (bridges). All excess tags and bridges were removed, resulting in sequence-specific tagging of mature miRNAs. The miRtagged mature miRNA was then hybridized to a probe pair (reporter probe and capture probe) in the standard nCounter gene expression array workflow. 200 ng of total RNA per sample (33 ng/μl per sample) was submitted for NanoString analysis, performed at the Fred Hutchinson Cancer Center in Seattle, Wash., USA. NanoString data was analyzed using nSolver 2.6 software. The data represents counts of molecules normalized against 4 housekeeping genes (β-actin, GAPDH, RpI19, and B2m), 8 negative controls, and 6 positive controls that were run simultaneously with the samples.

Single Cell RNA-Sequencing (scRNA-Seq)

Cultures of MG were dissociated from the plate and cells were pelleted at 300 g for 10 minutes at 4° C. Cells were then suspended in 0.04% BSA in PBS at a concentration of 2,000 cells per μL and loaded onto the Single Cell 3′ Chip (10× Genomics) with a targeted cell recovery of 4000 cells. GEM generation and barcoding, RT, cleanup, cDNA amplification, and library construction were performed according to the manufacturer's instructions. Single Cell libraries were sequenced with the Illumina NextSeq 500/550 v2 kit. Reads were processed in Cell Ranger (10× Genomics) and aligned to Mm10 and analyzed in Seurat using R-software. Data was analyzed and normalized by the percentage of mitochondrial genes and the number of genes per cell. The data was then scaled (by total expression) and log transformed. The scaled z-scores from Seurat's ScaleData function were used for dimensionality reduction and clustering. Linear dimensionality reduction was done using a principal components analysis (PCA) and the PCA scores are used for clustering. Non-linear dimensional reduction (tSNE) was used to visualize the cell clusters and explore components of the data. Differentially expressed genes were found using the default Wilcoxon rank sum test.

Fixation, Sectioning, and Immunofluorescent Labeling

MG cultures were fixed with 2% PFA. For immunofluorescent staining, cells were incubated in blocking solution (5% milk block: 2.5 g nonfat milk powder in 50 mL PBS; with 0.5% Triton-X100) for 1 h at RT. Primary antibodies (Table S5) were incubated in 5% milk block overnight, secondary antibodies (Invitrogen/Molecular Probes, and Jackson ImmunoResearch, 1:500-1,000) for 1 h at RT and counterstained with 4,6-diamidino-2-phenylindole (DAPI, Sigma, 1:1,000). EdU labeling was carried out using Click-iT EdU Kit (Invitrogen).

Microscopy, Cell Counts, and Statistical Analysis

Live imaging was performed using Zeiss Observer D1 with Axio-Cam. The number of living cells per field were evaluated for five random fields per coverslip, at 50× magnification using Image J. Fixed cells were analyzed by LSM880 confocal microscope and ZEN software (Zeiss, Germany). Three to five random fields per coverslip, at 200× magnification were counted and averaged for every condition. For high power images, pictures were taken at 600× magnification. Values are expressed as mean standard deviation (S.D.). Statistical analyses were performed by Mann-Whitney test or by t-test for independent samples combined with Levene's test for equality of variances, using SPSS software. Holm-Bonferroni method was used to correct for multiple comparisons. Images were processed and assembled with Adobe Photoshop and Adobe Illustrator.

### REFERENCES

Abernathy, D. G., et al., (2017). Cell stem cell 21, 332-348 e339.
Abrajano, J. J., et al., (2009) PloS one 4, e7936.
Andres, M. E., et al., (1999). Proceedings of the National Academy of Sciences of the United States of America 96, 9873-9878.
Balzeau, J., et al., (2017). Front Genet 8, 31.
Bardy, C., (2015). Proceedings of the National Academy of Sciences of the United States of America 112, E2725-2734.
Beveridge, N.J., et al., (2009). Cell Signal 21, 1837-1845.
Blackshaw, S., et al., (2004). PLoS Biol 2, E247.
Brett, J. O., et al., (2011). Aging (Albany N.Y.) 3, 108-124.
Brzezinski, J. A. t., et al., (2011). Development 138, 3519-3531.
Bueno, M. J., Malumbres, M., (2011). MicroRNAs and the cell cycle. Biochim Biophys Acta 1812, 592-601.
Busskamp, V., et al., (2014). Neuron 83, 586-600.
Das, A. V., et al. (2006). Developmental biology 299, 283-302.
De Brouwer, S., et al., (2012). Int J Cancer 130, 2591-2598.
Dennis, L., et al., (2015). WHITE PAPER NanoString Technologies 1.0.
Doeppner, T. R., et al., (2013). Acta Neuropathol 126, 251-265.
Elsaeidi, F., et al., (2018). The Journal of neuroscience: the official journal of the Society for Neuroscience 38, 2246-2261.
Foshay, K. M., Gallicano, G.I., (2009). Developmental biology 326, 431-443.
Gallina, D., et al., (2016). Developmental neurobiology 76, 983-1002.
Gao, X., et al., (2016). Sci Rep 6, 22490.
Geiss, G. K., et al., (2008). Nature biotechnology 26, 317-325.
Goldman, D., (2014). Nature reviews. Neuroscience 15, 431-442.
Grosche, A., et al., (2016). Mol Cell Proteomics 15, 462-480.
Haug, B. H., et al., (2011). Carcinogenesis 32, 1005-1012.
Hjelm, B. E., et al., (2011). Neurosci Lett 502, 219-224.
Hunt, S., et al., (2011). FEBS Lett 585, 187-192.
Huo, J., et al., (2016). Int J Mol Sci 17.
Idichi, T., et al., (2018). Oncotarget 9, 28849-28865.
Jadhav, A. P., et al., (2009). Progress in retinal and eye research 28, 249-262.
Jeon, C. J., et al., (1998). The Journal of Neuroscience 18, 8936-8946.
Jin, J., et al., (2016). Cell reports 16, 1653-1663.
Jin, J., et al., (2018). Biochem Biophys Res Commun.
Johnson, S. M., et al., (2003). Developmental biology 259, 364-379.
Jorstad, N. L., et al., (2017). Nature 548, 103-107.
Karl, M. O., et al., (2008). Proceedings of the National Academy of Sciences of the United States of America 105, 19508-19513.
La Torre, A., et al., (2013). Proceedings of the National Academy of Sciences of the United States of America 110, E2362-2370.
Lee, S. W., et al., (2018). Developmental cell 46, 73-84 e77.
Lee, Y. S., Dutta, A., (2007). Genes & development 21, 1025-1030.
Li, Y., et al., (2017). Oncotarget 8, 10274-10286.
Löffler, K., et al., (2015). Age-dependent Müller glia neurogenic competence in the mouse retina. Glia.
Makeyev, E. V., et al., (2007). Molecular cell 27, 435-448.
Mao, S., et al., (2014). FEBS J 281, 1144-1158.
Masserdotti, G., et al., (2015). Cell stem cell 17, 74-88.
Naka-Kaneda, H., et al., (2014). Proceedings of the National Academy of Sciences of the United States of America 111, 1604-1609.
Nakamura, R. E., et al., (2007). BMC Cell Biol 8, 52.
Nelson, B. R., et al., (2011). PloS one 6, e22817.
Neo, W. H., et al., (2014).The Journal of biological chemistry 289, 20788-20801.
Nesti, E., et al., (2014). Proceedings of the National Academy of Sciences of the United States of America 111, E3929-3936.
Pasquinelli, A. E., et al., (2000). Nature 408, 86-89.
Patterson, M., et al., (2014). Stem cell reports 3, 758-773.
Pollak, J., et al., (2013). Development 140, 2619-2631.
Qureshi, I. A., et al., (2010). Cell Cycle 9, 4477-4486.
Rajaram, K., et al., (2014). Developmental dynamics 243, 1591-1605.
Ramachandran, R., et al., (2010). Nature cell biology 12, 1101-1107.
Ramachandran, R., et al., (2011). Proceedings of the National Academy of Sciences of the United States of America 108, 15858-15863.
Reinhart, B. J., et al., (2000). Nature 403, 901-906.
Roesch, K., et al., (2008). The Journal of comparative neurology 509, 225-238.
Todd, L., Fischer, A. J., (2015). Development 142, 2610-2622.
Todd, L., et al., (2018). Stem cells 36, 392-405.
Trompeter, H. I., et al., (2011). PloS one 6, e16138.
Ueki, Y., et al., (2012). Glia 60, 1579-1589.
Ueki, Y., et al., (2015). Proceedings of the National Academy of Sciences of the United States of America 112, 13717-13722.
Vazquez-Chona, F. R., et al., (2009). Investigative ophthalmology & visual science 50, 3996-4003.
Victor, M. B., et al., (2014). Neuron 84, 311-323.
Visvanathan, J., et al., (2007). Genes & development 21, 744-749.
Wemig, M., et al., (2008). Proceedings of the National Academy of Sciences of the United States of America 105, 5856-5861.
Wilken, M.S., Reh, T. A., (2016). Current opinion in genetics & development 40, 57-64.
Wohl, S. G., et al., (2017). Nature communications 8, 1603.
Wohl, S. G., Reh, T. A., (2016a). Sci Rep 6, 35423.
Wohl, S. G., Reh, T. A., (2016b). Glia 64, 743-762.
Worringer, K. A., et al., (2014). Cell stem cell 14, 40-52.
Xia, X., Ahmad, I., (2016). Developmental biology 410, 70-85.
Xiang, L., et al., (2017). Proceedings of the National Academy of Sciences of the United States of America 114, 6376-6381.
Xu, S., et al., (2007). The Journal of biological chemistry 282, 25053-25066.
Xue, Y., et al., (2013). Cell 152, 82-96.
Yang, P., et al., (2017). J Neurosci Res 95, 1574-1581.
Yao, K., et al., (2016). Cell reports 17, 165-178.
Yoo, A. S., et al., (2009). Nature 460, 642-646.
Yoo, A. S., et al., (2011). Nature 476, 228-231.
Yoshida, A., et al., (2018). Acta Med Okayama 72, 165-174.
Yu, J., Vodyanik, et al., (2007). Science 318, 1917-1920.
Zelinka, C. P., et al., (2016). Development 143, 1859-1873.
Zhang, J., et al., (2015). Developmental biology 403, 128-138.
Zhu, Q., et al., (2011). The Journal of biological chemistry 286, 31749-31760.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A nucleic acid molecule comprising a nucleic acid sequence encoding Ascl1 and a nucleic acid sequence encoding one or more reprogramming potentiators.

2. The nucleic acid molecule of claim 1, wherein the one or more reprogramming potentiators is selected from the group consisting of one or more HDACi, one or more Jak/STATi, one or more Ascl1 activators, and a combination thereof.

3. The nucleic acid molecule of claim 2, wherein the one or more HDACi is selected from the group consisting of 16cyc-HxA, 161lin-HxA, 16KA, and a combination thereof.

4. The nucleic acid molecule of claim 2, wherein the one or more Jak/STATi is selected from the group consisting of Socs1, Socs2, Socs3, Socs4, Socs5, Socs6, Socs7, CIS, XpYL and a combination thereof.

5. The nucleic acid molecule of claim 2, wherein the one or more Ascl1 activators is selected from the group consisting of miR-25, miR-124, one or more let7 family inhibitors, and a combination thereof.

6. A method for inducing retinal regeneration in a subject comprising: a) administering to a retina of the subject a nucleic acid molecule comprising a nucleic acid sequence encoding Ascl1 and a nucleic acid sequence encoding one or more reprogramming potentiators, wherein Ascl1 induces neurogenesis from MG, and wherein the one or more reprogramming potentiators stimulate production of functional neurons from the Ascl1-induced MG.

7. The method of claim 6, wherein the number of the MG-derived functional neurons is increased.

8. The method of claim 7, wherein the number of functional neurons is increased by 40%.

9. The method of claim 6, wherein the subject is treated for retinal disease, damage or degeneration in the retina.

10. The method of claim 6, wherein the subject is an adult.

11. The method of claim 6, wherein a vector comprises the nucleic acid molecule, wherein the vector is a non-viral vector or a viral vector.

12. (canceled)

13. The method of claim 11, wherein the viral vector is an adeno-associated viral (AAV) vector or a lentiviral vector.

14. The method of claim 6, wherein a promoter sequence is in operable linkage with the nucleic acid encoding Ascl1.

15. The method of claim 14, wherein the promoter is a retinal or MG-specific promoter.

16. The method of claim 6, wherein administering to the retina is intravitreal or subretinal injection.

17. The method of claim 6, wherein the one or more reprogramming potentiating agents are selected from the group consisting of 1) miR-25, or an activator or mimic thereof; 2) miR-124, or an activator or mimic thereof; 3) one or more let-7 family inhibitors or antagomirs; and 4) a combination thereof.

18. The method of claim 17, wherein miR-25, miR-124, or the one or more let7 are expressed by a short hairpin RNA (shRNA).

19. A method for inducing retinal regeneration in a subject comprising: a) administering to a retina of the subject a polynucleotide sequence encoding Ascl1 (Achaete-scute-like family bHLH transcription factor 1), wherein expression of Ascl1 induces neurogenesis from Müller glia (MG); and b) sequentially or concurrently administering to the retina of the subject a combined therapy comprising a composition comprising one or more TSA/HDAC inhibitors (HDACi); and a composition comprising one or more Jak/STAT signaling pathway inhibitors (STATi), wherein the combined therapy stimulates production of functional neurons from the Ascl1-induced neurogenesis from the MG.

20. A nucleic acid molecule comprising a nucleic acid sequence encoding Ascl1 and a sequence selected from an MG-specific promoter sequence or a shRNA.

21. The nucleic acid molecule of claim 20, wherein the MG-specific promoter sequence is a Rbpl1 promoter sequence or a portion thereof.

22. (canceled)

23. The nucleic acid molecule of claim 20, wherein the shRNA is a miR-25, miR-124, or let-7 shRNA.


SEQUENCE LISTING (45 sequences)

SEQ ID NO: 1 - DNA, 2843 bp, Homo sapiens
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcaggtaggttgcattttggggtgggcaggggggtattcttgccttcgtcctccctctgagtgtctgtggaagtggggatgtctccaaggagataaggggatttttatttaaagaatttgtgaaagttggtcgatttcaagtcctagtttgttagtttcagcactggcctctgaaaatggccttgcccaggtctccaaggagtgaagggtagtagtgaggtgcagagatactggtgaaccgaatactgggacatgttaaaagagatgtctacctgacagactctttccccagacctccatctccctctaccactagcctacacgttcaaattaacctctcctgttcttttccttatgttatagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataa

SEQ ID NO: 2 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 3 - DNA, 2347 bp, Homo sapiens
tcagtactcagacccaagttgtgacttcctgtcaggaccaagaggcagcctgctgtggcgcaaaagttggacctgggttcaagttcttctcttccacttgcaggctgtgagatttaaccccagtcttggttgttccctgtaaagtaggatggaagtgttatgaaatgtaatggacacataagcaatgcttggttaccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatctgaaattcacatttaactgggtgtcctgtatttttatttgctaaatctgccatcaaattggtctggctcaacctggagaatggccaggtggtttcagttttacaggcatcttgggacgtggtctccctgctcccctgagtcctcaccccagtcctgggctccctggcccaggcaagccccaggtattgacttacctgccaggtcggcagctcctccttggggctacctggtacctgaatgtcctggagctctagaggttccctccgctggaggcgtggtccggtcagcaggttgggattagtgtgtcataaggaacttctcaccgcccacagtttccgttaaatcgggctcacaggaggccctcagtggggcaaaggaagacccagagagaaaggggagaggggagaggcctgggcctggctggaggcgcatcaaagccctcctttgtgtgctcctgctctggagttcctgctcggccatgtggaagcccggctgtggggctgggatctgggccagtcccattccctcttttctctgccctctttctcctcaagatcccggggtggggttgctgagagagcacccccccccccccaccaccaccaccagggtaataagaggtgaagggaaatcgtaaatatgactacatctacagtggcagctctggcaaatccaggcctattgcccacccctcccccagccagcaggacctggcatggtagttttcacc

SEQ ID NO: 4 - protein, 16 aa, Artificial Sequence
  Synthetic peptide
  MOD_RES (1)..(1): ACETYLATION
  SITE (4)..(4): IsoAsp
  MISC_FEATURE (4)..(7): cyclic peptide
  SITE (7)..(7): Diaminopropionic acid
  SITE (15)..(15): Hx
RRRDVAXREIRRYQXA

SEQ ID NO: 5 - protein, 16 aa, Artificial Sequence
  Synthetic peptide
  MOD_RES (1)..(1): ACETYLATION
  MISC_FEATURE (15)..(15): Hx
RRRTVALREIRRYQXA

SEQ ID NO: 6 - protein, 16 aa, Artificial Sequence
  Synthetic peptide
  MOD_RES (1)..(1): ACETYLATION
  MISC_FEATURE (4)..(4): IsoAsp
  MISC_FEATURE (4)..(7): Cyclic peptide
  MISC_FEATURE (7)..(7): Diaminopropionic acid
RRRDVAXREIRRYQKA

SEQ ID NO: 7 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 8 - DNA, 1216 bp, Homo sapiens
ggcagctgcacggctcctggccccggagcatgcgcgagagccgccccggagcgccccggagccccccgccgtcccgcccgcggcgtcccgcgccccgccgccagcgcacccccggacgctatggcccacccctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttt

SEQ ID NO: 9 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 10 - DNA, 2759 bp, Homo sapiens
agccgcggcctcaactaaaagtggccattgacctttcaagctttcgagcagtgatgcaatagaatagtatttcaaagaaaaatgcttatcgaaattttggatccggttttcccgtgattgttaagggtttcttttaaaaagtaggtcacatttcaagtaggtcatatttcgggggcgggtgcgcagacaaggagatgagtttccactaaggccagggggcctccaacggggttggaggtgagaatcccaggtagggtagaggtgccgagatccttccgaatcccagccctggggcgtcagccctgcagggaatggcagagacactctccggactgagggaaccgaggccagtcaccaagccccttccgggcgcgcaggcgatcagtgggtgaccgcggctgcgagggactttgtcatccgtcctccaggatctggggagaaagagccccatcccttctctctctgccaccatttcggacaccccgcagggactcgttttgggattcgcactgacttcaaggaaggacgcgaacccttctctgaccccagctcgggcggccacctgtctttgccgcggtgacccttctctcatgaccctgcggtgccttgagccctccgggaatggcggggaagggacgcggagccagtgggggaccgcggggtcggcggaggagccatccccgcaggcggcgcgtctggcgaaggccctgcgggagctcggtcagacaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctttatctgaccaaaccgctctacacgtcagcaccatctctgcagcatctctgtaggctcaccattaacaaatgtaccggtgccatctggggactgcctttaccaacaagactaaaagattacttggaagaatataaattccaggtataaatgtttctctttttttaaacatgtctcacatagagtatctccgaatgcagctatgtaaaagagaaccaaaacttgagtgctctggataactatatggaatgctttctaagaacagctgaagctaatctaatttaaatttaacagcttgaagaggtagctaggtgtttaaagttcctccagatacttttacctgagtgatgcttcccttcctaaggctgaccaagacctgttgatccttttagattaaaaataaaatgtcgcatgtaaaggctgaagtcgcgttttatcagaatgccttgccttcttaggttcttttccattatgtcaaaggtccaggctccagtaggagagaaagaactcctcataggaatactgaagaagtgggaaggaaccaagctgacacaggcctcactgcaatttgatatgcctgctgatcagagtctcttgggcattttatattttgcattctgatgtacctaggagttttgttaaacagatgatgtatgtgagtatttatcccattttatgcaattaaccaaatcaaccaaaaaaagtgaccatgaagtcctgtatttgtctttttactacatgtaggaactctcatgtgaatgagtactgtagtaatccattctatgggagccttatttcagaaatatttcaaactggtgcaaatggaaaagactttctcttttcctttaaagctaaagacaagaatatcatgctatacaggtgcaactcaatccccgttaataaaaaccaatgtaggtataggcattctaccctttgaaatagctgtgtcccaacctgttgccattgattttttggaaatggctttagaaatatccaagttgtccttgaattgtctaaccatggacataaacagttgtctcccttctactgtgtagaatactttgacttaattttcttccagatacagggggatacctgcctgtttttcaaagtgtttatttactgctgttactatttgattagaatgtattaaataaaaaaaacctgatttctacaagttgcacttattgagttctagagaacgtacactttcatggtaatagaggattgccataaaaacttacgtcaagtgaaataagccaattattcaacaaaaggtagaacattacttgccattctgtaaagttatgggctgtacctgccccctttgcaatttggaaagcatggtttagaaactacaggcattgtcaagtggccgggtcttttataatttgaataggcataacactgatgtcctctgtgtttccaaaaacatggtttagaaactacaaacattatgacatggccagtcttttacaagttgagtaggcataatactaaagaaaaatacaaagttttgtggccacttattttttgctatgttagtctgcataactgttataaatgtaccatcttttctagagtccagacattatttattttatggctttaaaattttcctgcatagctacaatcctgtggtgtgtcaccataaaggtggaccctgtgtgaatgagaaaattcagttataaattgtaataaaacctgcttactggaaaaaaa

SEQ ID NO: 11 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 12 - DNA, 2737 bp, Homo sapiens
gcggctccgacttggactccctgctccgctgctgccgcttcggccccgcacgcagccagccgccagccgcccgcccggcccagctcccgccgcggccccttgccgcggtccctctcctggtcccctcccggttggtccgggggtgcgcagggggcagggcgggcgcccaggggaagctcgagggacgcgcgcgcgaaggctcctttgtggacttcacggccgccaacatctgggcgcagcgcgggccaccgctggccgtctcgccgccgcgtcgccttggggacccgagggggctcagccccaaggacggagacttcgattcgggaccagccccccgggatgcggtagcggccgctgtgcggaggccgcgaagcagctgcagccgccgccgcgcagatccacgctggctccgtgcgccatggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaaggggtaaagggcgcaaagggcatgggtcgggagaggggacgcaggcccctctcctccgtggcacatggcacaagcacaagaagccaaccaggagagagtcctgtagctctggggggaaagagggcggacaggcccctccctctgccctctccctgcagaatgtggcaggcggacctggaatgtgttggagggaagggggagtaccacctgagtctccagcttctccggaggagccagctgtcctggtgggacgatagcaaccacaagtggattctccttcaattcctcagcttcccctctgcctccaaacaggggacacttcgggaatgctgaactaatgagaactgccagggaatcttcaaactttccaacggaacttgtttgctctttgatttggtttaaacctgagctggttgtggagcctgggaaaggtggaagagagagaggtcctgagggccccagggctgcgggctggcgaaggaaatggtcacaccccccgcccaccccaggcgaggatcctggtgacatgctcctctccctggctccggggagaagggcttggggtgacctgaagggaaccatcctggtaccccacatcctctcctccgggacagtcaccgaaaacacaggttccaaagtctacctggtgcctgagagcccagggcccttcctccgttttaagggggaagcaacatttggaggggatggatgggctggtcagctggtctccttttcctactcatactataccttcctgtacctgggtggatggagcgggaggatggaggagacgggacatctttcacctcaggctcctggtagagaagacaggggattctactctgtgcctcctgactatgtctggctaagagattcgccttaaatgctccctgtcccatggagagggacccagcataggaaagccacatactcagcctggatgggtggagaggctgagggactcactggagggcaccaagccagcccacagccagggaagtggggagggggggcggaaacccatgcctcccagctgagcactgggaatgtcagcccagtaagtattggccagtcaggcgcctcgtggtcagagcagagccaccaggtcccactgccccgagccctgcacagccctccctcctgcctgggtgggggaggctggaggtcattggagaggctggactgctgccaccccgggtgctcccgctctgccatagcactgatcagtgacaatttacaggaatgtagcagcgatggaattacctggaacagttttttgtttttgtttttgtttttgtttttgtgggggggggcaactaaacaaacacaaagtattctgtgtcaggtattgggctggacagggcagttgtgtgttggggtggtttttttctctatttttttgtttgtttcttgttttttaataatgtttacaatctgcctcaatcactctgtcttttataaagattccacctccagtcctctctcctcccccctactcaggcccttgaggctattaggagatgcttgaagaactcaacaaaatcccaatccaagtcaaactttgcacatatttatatttatattcagaaaagaaacatttcagtaatttataataaagagcactattttttaatgaaaaac

SEQ ID NO: 13 - protein, 440 aa, Homo sapiens
MAENNENISKNVDVRPKTSRSRSADRKDGYVWSGKKLSWSKKSESYSDAETVNGIEKTEVSLRNQERKHSCSSIELDLDHSCGHRFLGRSLKQKLQDAVGQCFPIKNCSSRHSSGLPSKRKIHISELMLDKCPFPPRSDLAFRWHFIKRHTAPINSKSDEWVSTDLSQTELRDGQLKRRNMEENINCFSHTNVQPCVITTDNALCREGPMTGSVMNLVSNNSIEDSDMDSDDEILTLCTSSRKRNKPKWDLDDEILQLETPPKYHTQIDYVHCLVPDLLQINNNPCYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPLIRTFPFSLQHICRTVICNCTTYDGIDALPIPSSMKLYLKEYHYKSKVRVLRIDAPEQQC

SEQ ID NO: 14 - DNA, 6903 bp, Homo sapiens
acggaagtggtggcggcgcccagggaaccggcggaggcgatgaccgtgacggctgggttgggaccggaacgccgaagcggggttgggggtggcagaaaagcatctgctttgtaagacctacacgaggtgcaggagtggttgggcctcccctctccacttaagcaagcgcccagactgatggcgatggtgatggcagcagttactcgcacaaccccagttaagctgcgctccgggaggagttcatctgttgtctgctctccagtgacttccgtttgtggagcctaagtgtttctacagaatccaactcaaatatcaaggactatgatcagaatgctttcaaggagtttttgggcacatgatggcagatacatccagaaagtgcccagaagaaacttcctgctggaaaaaatgaaaaagcagtatttataacattagaatctggataatttgttaacatggcagaaaataatgaaaatattagtaaaaatgtagatgtaaggcccaaaactagtcggagcagaagtgccgacagaaaagacggttatgtgtggagtggaaagaagttatcttggtcaaaaaagagtgagagttattcagatgctgagacagtgaatggtatagagaaaaccgaagtgtctttaaggaaccaagaaaggaagcacagctgttcatccattgagttggacttagatcattcctgtgggcatcgatttttaggccgatctcttaaacagaaactgcaagatgccgtggggcagtgttttccaataaagaattgtagtagtcggcactcttcagggcttccgtctaaaaggaaaattcatatcagtgaactcatgttagataagtgtcctttcccacctcgatcagatttagcctttaggtggcattttattaaacgacacactgctcctataaattccaaatcagatgaatgggtaagcacagacttgtctcagactgaattgagggatggtcagctaaaacgaagaaatatggaagaaaatataaactgtttctcacataccaatgttcagccctgtgtcataaccaccgacaatgctttgtgtagagaaggtcctatgactggctctgtgatgaacctggtttcaaataacagtatagaagatagtgatatggattccgatgatgaaattctaacactttgcacaagttccagaaaaagaaacaaacccaaatgggatttggatgatgaaatcctgcagttggaaacacctcctaaataccacacgcagattgattatgtccactgtcttgtaccagacctccttcagatcaataacaacccatgttactggggagtgatggataaatacgcagccgaagcactactggaaggaaaaccagagggtacctttttacttcgagactcagcacaggaagactatttattctctgttagttttagacgctatagtcgttctcttcatgctagaattgaacagtggaatcacaactttagctttgatgcacatgacccctgtgtcttccattctcctgacattactgggctcctagaacattataaggacccaagcgcctgtatgttctttgaaccacttctatccactcccttaattcggactttccctttttccctgcagcatatatgcagaacagttatttgtaactgtacaacttatgatggcatcgatgcccttccaattccttcttctatgaaattatatctgaaggaatatcattataaatcaaaagttagagtactcaggattgatgcaccagaacagcaatgctagtaacaggatgggaacatgggaatgataatatatattttttcttttaatattttatttttctttttatgccactttggatttttctacaaaggcagtggtgtccaaaataaaatctctgccctaaattttactaataaatccatttttctagtgatacacaaattgtttaaggttatacactcgagcttaaatagatatttttaaccaggtgtttggtttttgtttttaccgtgtaggttgtatacttacattttttctttccttaatttatacatgatcctttttccttaatttatacatgatccaggcacatttgaaatttggtaggcattgcaaacacatttgaatattgtgtatttcatactattctttaaaagtaatcttatgtcctttcctacatgtaaaatattttgttaatctatgccattagtagtattatatataaaatatagttccccgtcccccttttcattgagtttgatattcttcagtaaagctgaatgttgtaattcaccattcatactaatgttattgacttttgtaacttactaatagggatgttaaaagtaacaaaaccaagtcaaacttggtgtatttttattttaaatattaactctaaagcaggattactaaatttgattaatctggtcaatgagaatcaaatggaaacatatcattctggaggtgcacttactcttctacaatgtggccggatcatttgtcattcctgaataggtctttctctcaacctgcaagagctaaaccatcttcaaaacataaatcttgttcctttctcattggaagctttgaaaataatgataacactattagtagctagtagttactaaagtgctaaagtaattgtggggtttttctgtattagcttatgaactgttgcttctacttagcccttttatagaaactctgagctgttactttggggaaattccacaatgtattagcagccacaaatttccactggtaaccaaagagtacctaagtagtttttcattattttaagttgaatttgaataaagattccagaaataaacatggagctcagtattcaggaagcttaatactgtttttgatctacaagatccaaataattatccctatcttcagaattaacttagtctgtaatgtggcttcaaaaaaagaaagccttttgacagctacatgacttacaccatacagaacagtactggacaaaaggggtgatattttaaatttactatcccagaaacagggcaacgtttttagaaacatctttagcccaaagtaactagtaaaaataaatggagtggtatcctatcttctttttttaaggaatcaatgaataataaatgtagatagacaattttcttctggtgagctgtctaaccctttgtacacattgacatttttaatatcgtaactgatttcttgatgctagtttagctatattaggaaactgcttctacctagattgaaagaaatttgactcataaacttccaagttagaacaaatatttcttcattattgttgcttttatgtgagtagcatttccctatcttgcagttctgttaacatgaaaatggcatttttccataatagctttaaaataattttttagtaattattacaaaattaaggaaaatctctattaccgttatcttttagcattttttttttcctcagtgatctcaagattgtcttagtctcaatgagatgtagctacaaaaatggcacctcttagcagtgatgaggctcaggatatcggatatcataacaccaatgtcaggataaatatctcttctaaagacgatatttaccttttacaaggttagaaaagtctcatactacctcatctttattgtggcgcttttgtagatcactgagaagcttatcttattaaccaatataccacttcctaaatatccatctttggtgaaagaaaatagtgtggtaagagtacccatgattatagttttttatcagaatttgataagattttctgtttctgtgaaacaattattttaaatatttttcttttaaaaataactttttatcagtacagaaaaacctaagggatgactagcttacaaatattctgttaaggggtagttttataaagaaaaatgatatatagttatgttgtcttttatagtgttaaaattagtatttatatgaaagtcaagatctaagtaccttttcatataattcaaactgattgcttgtgatatgttttctctggcttttttattttttcaacttcaaggtattaacagctgttaacacttaaaaagtactgccacatatgtagccttgaagtagttgatctaataaatgagagcagggaggagattcattgactctgaaatgatttttatttttcctttggtatttgctacagtgaaaagaagtttggaaagtagatacataaagacttgagaagggaattgtttaagggaaaagaaaagagtaataattttttttaaaacattaaggaattggaattcagaaaggttacccccttcctctgtgtctttgtaaggatacatttggaagaacatacagtattcagaaactaacctatataaaatgtaaacaatatattgattgcactatataaatgagtaaattttgagaggaacaatgtgggaatgtataaatttgtctaaaattttataactgttgaacaaagggaagatttgaacagtgtaaattccagagttgagaaagcatttagcatggtggaaatgtggagagcttattggtgagagaggatggagctaagagaaataagatcaaatattaaaattaggaatttaggcattacctcagggaaaagccttgacagtgaaaagaaactacttaatcgctaatagctagtctggaccagacacttaactgcgctttgggaatcatcctgggctaatcaggaaataaacttgacttttctatttttctctgaagctttacaggacatactcccactcccatttttttctaagcctacagcatcttgattcatagtataattcctgaagtatgctgaatggactttttaaggttcctttagcatttttgtatagagttatatgtgatctttctataaccatgccattcacttaaataattcatgaagggaatggtttaatttaaaggaagacctaataggaagaagattaaaggaagcttttagtcagcctgtggctagatttattgatttggtgatcaaacctgtgctatttcctgaatttcagctaatggccaactgtggtcaaagaatggctttcagttaaagttttgacttcttattataaataatataaagaatgtctcagtaattagaaatcccttaataagtagaacaaatgtggcaagtaggactcctatcggtatacaaattccatttctttttataagaaaaacacataaaccattatcatttattcatgagccaaagccattaagaagataaatccagttatcagcttcctgtctctgaagtttggatcattttatagatccacgcaatagagagcttccttcctatgaaacaaaaaccttgagatctctttttttttgagacagagtctcgctgttgtcagcccaggctggagtgcaatggcgcaatctcggttcactgcaacctctgccttccaggttccagcaattctcctacctcagctcccaagtagctgagatgacaggcgtgtgccaccacgcccagctaatttttgtatttttagtagcgatggtgtttcactacattggccaggctggtctcgaactcctgacctcaggtgaccacccgcctcagcctcccaaagtgctgggattacaggcatgagccactgtgcccggccaagatctaactcacgcttacttttcattaaaatatttcttgtcgcttttagctattgtcagaatccaataaacagcttacgcattcattttggtaaagcaaatttcacaggaaaatagacaaaattataagagattcctggagacaacacaaggaaaacaatgtttaaaagcaaaatagcctgtgtaatgttaggtaatgtaatgcccaagtgaataaaagatttttcgtcaagcaaactactgtttctactttgggggaaaaaagtcagttttacatttgtaatttaaggaaagaacagaaagcacaaaggtttttcttttctttattaatggcctactagaaatgagcagtgcaagagtctacctgtactattctaatacagtaagatattggacacaaaatggaggtaactttttaaaatagattggcttggaagttgaaatgtagattaatggatataacccaatagaaagggattttcaaataaaaccaaagtctatttttttatttactttctaattttgaacagatgactcagtcctaaatctttgcctttatttcctataaaatgtaggtgatacttgtaactcgacttcctggagttaattcttcagcaagaggtgaccctgggagcgtactttaaaataattgctgtacagccattgagtactaacatgatgataggttttcaaaatatctttgtagtggatgctgcataattacattcacttctcttagactgtaaaagactttcttgacttgttttaacagtagagatagcagtacaatttgaatttatggtttaggctctgcaattagaggaacaattgcagtttcctcctacccttcatatggtctgtgtaaaactgatgtttgcttaacttattttaaaagttgattacgttttcagaaaataaagataatcacttttgccatggttataatcaaatctaagctttcagacttgagagccatggtgtaaaactcaaggaggtttatttaaattatgctgactttgctagaattggataaattctgtataagccaagtatgagttcacatgtactcgaatatacagttttcacaaagctattactctcatcagtcaggcttgtatgatctattccttaccacaaaagaagtagacaattgccacttttatttctaatccttaagttgaatgtttcttcttggatgtaagttcaaataaattgatctggataaattttcatttctacttaattaaaacttcctatgtaaaatccaaa

SEQ ID NO: 15 - protein, 536 aa, Homo sapiens
MDKVGKMWNNFKYRCQNLFGHEGGSRSENVDMNSNRCLSVKEKNISIGDSTPQQQSSPLRENIALQLGLSPSKNSSRRNQNCATEIPQIVEISIEKDNDSCVTPGTRLARRDSYSRHAPWGGKKKHSCSTKTQSSLDADKKFGRTRSGLQRRERRYGVSSVHDMDSVSSRTVGSRSLRQRLQDTVGLCFPMRTYSKQSKPLFSNKRKIHLSELMLEKCPFPAGSDLAQKWHLIKQHTAPVSPHSTFFDTFDPSLVSTEDEEDRLRERRRLSIEEGVDPPPNAQIHTFEATAQVNPLYKLGPKLAPGMTEISGDSSAIPQANCDSEEDTTTLCLQSRRQKQRQISGDSHTHVSRQGAWKVHTQIDYIHCLVPDLLQITGNPCYWGVMDRYEAEALLEGKPEGTFLLRDSAQEDYLFSVSFRRYNRSLHARIEQWNHNFSFDAHDPCVFHSSTVTGLLEHYKDPSSCMFFEPLLTISLNRTFPFSLQYICRAVICRCTTYDGIDGLPLPSMLQDFLKEYHYKQKVRVRWLEREPVKAK

SEQ ID NO: 16 - DNA, 4422 bp, Homo sapiens
cgcagagcgcgcacccaagcggccggacctccccgactcccgggccgcccccgtctcgcgccctgcctccctccttcggccttcacctacccgcctccggattggccgctaggaatcccgcccctctaaagccctgcctgctattggtcacgattttataatcaatggataaagtgggaaaaatgtggaataacttcaaatacaggtgtcagaatctcttcggtcatgagggaggaagccgtagtgaaaatgtggacatgaactccaacagatgtttgtctgtcaaagagaaaaacatcagcataggagactcaactcctcagcaacaaagcagtcccttaagagaaaatattgccttacaactgggattaagcccttcgaagaattcttcaaggagaaatcaaaattgtgccacagaaatccctcaaattgttgaaataagcatcgaaaaggataatgattcttgtgttaccccaggaacaagacttgcacgaagagattcctactctcgacatgctccatggggtgggaagaaaaaacattcctgttctacaaagacccagagttcattggatgctgataaaaagtttggtagaactcgaagtggacttcaaaggagagagaggcgctacggcgtaagttctgtacacgacatggacagtgtttccagcagaactgtaggaagtcgctctctaagacagaggttgcaggatactgtgggcttgtgttttcccatgagaacttacagcaagcagtcaaagcctctcttttccaataaaagaaaaatccatctctctgaattaatgcttgagaaatgcccttttcctgctggctcagatttagcccaaaaatggcatttgattaaacagcatacagctcctgtgagcccacattcaacattttttgatacatttgatccatctttggtttctacagaagatgaagaagataggcttagagagagaaggcggcttagtattgaagaaggggttgatccccctcccaatgcacaaatacatacatttgaagctactgcacaggttaatccattatataaactgggaccaaaattagctcctggaatgactgaaataagtggggacagttctgcaattccacaagctaattgtgactcggaagaggatacaaccaccctgtgtttgcagtcacggaggcagaagcagcgtcagatatctggagacagccatacccatgttagcagacagggagcttggaaagtccacacacagattgattacatacactgcctcgtgcctgatttgcttcaaattacagggaatccctgttactggggagtgatggaccgttatgaagcagaagcccttctcgaagggaaacctgaaggcacgtttttgctcagggactctgcgcaagaggactacctcttctctgtgagcttccgccgctacaacagatccctgcatgcccgaattgagcagtggaatcacaactttagtttcgacgcccatgacccgtgtgtatttcactcctccactgtaacgggacttttagaacattataaagatcccagttcgtgcatgttttttgaaccattgcttactatatcactaaataggactttcccttttagcctgcagtatatctgtcgcgcggtaatctgcaggtgcactacgtatgatggaattgatgggctccctctaccctcaatgttacaggattttttaaaagagtatcattataaacaaaaagttagagttcgctggttggaacgagaaccagtcaaggcaaagtaaactctccggtccccaaaggttgttaactaggtccgctttcatgtgcatcagacagtacacctatagcaagcacacgtagcagtgttaggctttttcatacagtatgtaagcttagtgttagtatctgtcagatgctacctgctgttacttattcagataaacatggtgcctattggaacaatagcggatagagctacaggtgttcagtaagactacaaaaacattttgcctatttcgctaacagtttggtttttaatggctgtggtatttgagtgaggcaactctggggcatttgttatgaagaattctatttcttactgaagaacaaattattaatattggatgagtatttcaacagtgtgactaatgtttgaaattattttttctaagagtttttctataaccttccaaaagtcgtgatgtttgtagttactataaatcaagctttggaagtccaaaaagaataaaagactgccttccttttagaaaaaaatgcaattttctggccacaagggcatagtgcagttcacttacgtgttgatgtagtttataatcagacgccttttctcttctgcaaaaggtactgttaagtaaaccagattttctaaataggcattcttaaaatttcagacttacaaagctagtagtagaattttattgaaaggcctaggtattaattttttaaatgagtgctttaacttaaaacaggcgtttggaatagctgctgcaatgtagtcttgtgtgtgatttttttttaagttgatgtgcagtctaattgttgtttcataaaagttggatctgttcctatgcccaggatgattttgtgaaccgtgaagtacgtgagactagaagacgcccaaacaagtcagataatagtaactacaatggttgctgatgttgagattattgttgaactataattaataatttggatggcagaatttatctcttttttgtaaactctcataactgaattgcttaagtataatttatagaatttcagtgcagttcattcttaatggaaaatctgaaacctaaattgcagatttaaaaggtactgtacaaccattatatctgtaaataacttagcacctttttgtcacttagaataatatgtactactacttgagtgagcgcttttggaagttatatcaagttctagtgtttgcttcttagtaactgaactgaatttacagttctgtcctagacattttgcactaaagtagccgaatccactctcatgtcttttcgttaatgtgctctgtaccactggtgagtgctccatagtttccttacctgctgctacagaatgttattttacatccctatggctattgccaaggctacaaaaaaggaaagctatatttgtatgcaacactaaccttttgactgctaatgtatgtttctgcttgctgtgccttgttatggctgctttttttgtgctaataaagtatgtttggtgttctccttgtatatctgctgttttatacatttgcaacaatttctcttgtaaatggaatggtttggggtttttaaataagcattaactaacaacctttctatagttaatgcagagttaatgaacagtctaatattgacttatcagaataagctaactctaaatttaatgctctacatcttatcagtcataattatatatactgtggaacagtatctgtagttactgcaaattactgtacagtttaggttataacagaaaactgacagagaagtaataaacctattgatttctctgcttataaatgaaagattgaaactatccaatgacatattatagtaaatgagtatctgtaacctcccactgcatcagaagcaggttaaatgaagtcttgtgaatttgtaatagatcagtaccatttattggtttggggaccatcttaattaaaaataaatgcccaaaatgtagaactttaaccaaagacttgtcccttttaaagcaaaatggggattgaagggacttataatttctgttgtttctaattaaagtccctgaagatcatataccaaagtgtttgagaacttcatccaaacctactttaaagcattatgtgcaattaagttgttatgacataattatattgcctaattgttgggtcttttttcttgagcttataatgtacctggaaaataaacctcttgagaaaaagaaaagttcatactgattattggaaaaggactatatatgtgagcaagattgtgttttagagaggaaacttgaaactccaagaaagcacttgatgtttttatatgcttgtagcaaattgatgttctaactgtagttttatagaaagtattaatgcttttatgtatttcaaaactttcatatgttaaatggaaattgttttaaatgtgtttgagtttatgtaagcatgtatacactgtgctaaaagtcacatgtttcagtttgtgtataatattaatatgcaatttttggtttaaatttttgtcttaaaatattagtggcttacattttaaaaaagaaaaatcaccagcatgaacttgcacctaagtctatattcactgtgtccttttctgaatcccattgtagcctgtcaactaaatttgagtgttaacggtctttttaaagtgcatttaaatacaaaccaggaatttctttagaagttga

SEQ ID NO: 17 - protein, 535 aa, Homo sapiens
MKKISLKTLRKSFNLNKSKEETDFMVVQQPSLASDFGKDDSLFGSCYGKDMASCDINGEDEKGGKNRSKSESLMGTLKRRLSAKQKSKGKAGTPSGSSADEDTFSSSSAPIVFKDVRAQRPIRSTSLRSHHYSPAPWPLRPTNSEETCIKMEVRVKALVHSSSPSPALNGVRKDFHDLQSETTCQEQANSLKSSASHNGDLHLHLDEHVPVVIGLMPQDYIQYTVPLDEGMYPLEGSRSYCLDSSSPMEVSAVPPQVGGRAFPEDESQVDQDLVVAPEIFVDQSVNGLLIGTTGVMLQSPRAGHDDVPPLSPLLPPMQNNQIQRNFSGLTGTEAHVAESMRCHLNFDPNSAPGVARVYDSVQSSGPMVVTSLTEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGSATYPVRLTNPVSRFMQVRSLQYLCRFVIRQYTRIDLIQKLPLPNKMKDYLQEKHY

SEQ ID NO: 18 - DNA, 5703 bp, Homo sapiens
agtcgcgccggcgctgggcgaggaagcggagccgggccgcctccggaaaatggctccaaaggttaaatgaagcaggaaaaatacatagatgcagccttgcagcctctccagatgtttggggataatattccagatagaaatattgatcccttggattaggtaactagtcataatgaagaaaattagtcttaaaaccttacggaaatcttttaacttgaataaaagtaaagaagaaactgatttcatggtagtacaacaaccatcgctagccagtgactttggaaaagatgattccttatttggtagctgctatggtaaagatatggccagctgcgatatcaacggtgaagatgaaaaaggcggaaaaaacagatcaaaaagcgagagcctgatgggtacgctaaaaaggcggctttctgcaaaacagaagtcaaaaggcaaggcgggcacaccctctgggagctctgccgacgaggacaccttctcctcctcctcagcacccatagtctttaaagacgtgagagctcagaggccgataaggtccacgtcgctccgcagccatcactacagtcccgcgccgtggcctctgcggcccacaaactccgaggagacctgcatcaagatggaggtgagagtcaaggccttggttcactcttccagcccgagtccagccctgaatggcgtccggaaggatttccacgacctccagtctgagaccacgtgccaggagcaagccaattcactgaagagctcggcttctcataatggagacctgcatcttcacctggatgaacatgtgcctgtcgttattggacttatgcctcaggactacattcagtatactgtgcctttagatgaggggatgtatcctttggaaggatcacggagctattgtctggacagctcttctcccatggaagtctctgcggttcctcctcaagtgggagggcgcgctttccccgaggatgagagtcaggtagaccaggacctagttgtcgccccagagatcttcgtggatcagtccgtgaatggcttgttgattggcaccacgggagtcatgttgcagagcccgagagcgggtcacgatgatgtccctccactctcaccattgctacctccaatgcagaataatcaaatccaaaggaacttcagtggactcactggcacagaagcccacgtggctgaaagtatgcgctgtcatttgaattttgatccgaactctgctcctggggttgcaagagtttatgactcagtgcaaagtagtggtcccatggttgtgacaagccttacagaggagctgaaaaaacttgcaaagcaaggatggtactggggaccaatcacacgttgggaggcagaagggaagctagcaaacgtgccagatggttcttttcttgttcgggacagttctgacgaccgttaccttttaagcttgagctttcgctcccatggtaaaacacttcacactagaattgagcactcaaatggtaggtttagcttttatgaacagccagatgtggaaggacatacgtccatagttgatctaattgagcattcaatcagggactctgaaaatggagctttttgttattcaaggtctcggctgcctggatctgcaacttaccccgtcagactgaccaacccagtgtcccggttcatgcaggtgcgctcgttgcagtacctgtgtcgttttgttatacgtcagtataccagaatagacttaattcagaaactgcctttgccaaacaaaatgaaggattatttacaggagaagcactactgaaagattgagaaccctgcatcttgcactttgggaataagaacaagagattgaaatacagtttacaaactttcattgccatcaaaatcttttgctgccataactatttcagttttatgtgtaaaagagtcatcagtttgtttaggggtggggaagtgtcagcaaggtgtcttgggtttattttggttctttaaaaaagggaagtcttgaggttttagaggtgtgaattatgtttcatcaatgtgcagaataatcacaatgtgaattatcaaattctcctcaatgccccccccgcccagtcctttgctgctatccactgtgatttttatgcattaaaagcacatttcatgtgtattcaaccctaagtaaagttgaatgaaacttaacagaatggaaattgctatgtctttttaaatggtccattttcaaaagacagtgttgaataaacatacctgtgtgataaaacacagaatttacatatacactgaagatgagtttttaatctcttactttaaaaagatttatttagaatcgtgaattgacataatcttgggtaatggaacggagatctgcaacatatcttttaacaacacttttttctaaattatttctaaggttgtgctaattcttttggttgtgaaaagttgaatttttctgttgccttcgttttcatcttctagtttgtctattttaataaatggccttacattaaaaaattgtaaagaaatgtataccaccaatttagaaattgttgccttttctgtaattaaactcgggtacaaattggcataacatgaaaacctatggaactagaattattattaaagaaatattagatgatcatagcttcctgtgatagcatttttttgtgtgttacctatccttttggtaaaatgttttatctgtgatttctttagcttagtcaacattttcttgggtgaacttgattgtcaactaattttcataaatggactggattctcttgcaacattaatgtttataaaaagttttaaattgatttgaatagaaagaaaacattgttttaaagttggatttatatttttcttctatgtagttactataaaagtgtgctggatttgaccaatccttacccccactataaagagaacccgtgatgactttagtttaaaaattgtggaaattgtggagcaatttttctcacaatgtgagaaaaattctaaaccatattagataatgtggaagtcatattgtctatcatatatactgccatttaaaaataggtttttaaaatttagctaagtcttaagtaatttgccgttgctaataattttatctccttgagtcggttgttggggagagatgttatattcaataatttttagttattttgtaatgcagagtgtttattcatttcacagttctgcaatggatgtagtattttgggattgccctgtccagaaaattttcagctacacacctttaaaggaaaatgtttctatctcagatgaaacatgtaatttgggatggttcttcctttgtcacttaaaggaagagataggaaaagtctcttacccactttaaacatgagggtaaaggtttaggtcaaacttactggctttgtcattcaagcatatctgaatcctcacttttttctctttgctttttagggtcagaactgagatattaccaagaaaaggcacaatgccataatattatggtgttatggtattttgacttaaaggggaaaaggtacttaattttggtgggatgttgattgtaccttgttaaaaagactctcattttctcatatgttttctcctaataagatggaatatggagtatactgtaataatataagtgttcattataagctatttggattaagaactattgcagagttgtaagcttgttatcaaattaatgcaagacatttaaactatttttttgcaaaactatttatttttaaaacaacttaaagtatatctagggtgagttaaaagtccctgtgcatctatattagatggcaggttttgtcacagagtcactgtgtattaataataaatgttgaaatggcttctccgtgtctccagaagcatttacatgtcctccttgtgagatcatggtgcacagaggtctttggactgccctgaacccgtcttatgtggacataacctattcccttcgttttctcatcatgccatgtgtttaagatctacccgcttagtgtcaagattattgagattttcatctaaatgtttttaaaattgtattttgtgttttagtggaggacagtcttgatactgttctaattcagaaagccaaattttgatgcgcttttgtatattcataatatatactttaatatgccctattctaatctagtttgaaattgttagattctgatagtataatgataaaatcaaactatttgccaaaaagttaaatttacaagcagaaagatgttttgcgatattttctacttttgtgtagaaagataaccatttggggtaggcagcacaacagaatgtgaaagattggctttatatgatactaacacagtccagttaaaagtggaaacagggaaaagagagagagaaaactcatcatttagtttggtctagacacaatttggttaaattttaggattctattggcagttaacatagatgacatggacacagcctatgagtgctttttaaatacgtgcttctaaaaatctgagctcaaagcaagaaaaagaaaacaggaaagctgtatccatttgccataatagatgaatcagaattgttttctattaaatatttcatcacgaaatatgatggaggccagaagctatttttaggcttactataacactattatgcatttttataaataagaagaaggctgtagaaagtacttgaaaaatatctaattctctttctgtctaaggaaaactctttctacttggtgcaataatctgaaaatttagaaggtcaaaatatgtcacaggaaatgatcatagacctaaaaatagtttttaaggaaaccagctacacggggcaaccaaccatcttgctgttgaagaaaacagttccaaaaggctaaatcattggcataaaggaaacaaaacccaaagtatgcgttgtaattcttgagccagtattactgaaacaggataaaagttataaaatctaacataggttaatgcaatgtctctgctagtgctacaagtctaaaatatctttgtaacaatgctttcagaaatgccagttttaattacatttcacattgtatatgagagatactgctttatgaatgaatcggaataatacattttcatttaaatcttaattgcttttcattaaatgcgaagtcttaatttcaaaatgaaatcacactaccagcaatagacagttttcttgaaaactctaataaggaacaatagccagttccgtaaataactttaaaattctaaaagtgttggtacactagcaatcacaaatatggtttcttttaatacttttttaatcctgtaaagaagggtttttataaacattctttttattaatcagtcataacatggcgaagtgtgtagttgctgtttctgaaaagtgatccaaactctacatccagagattatgaaaaattcttatagaattttgtaacaagtatttacatgttgggtgaaagaaatttcatagtcgttggagtgccatgaaattaatacttgcaattcagattgcggtagtttacactttttctgtatgtttcaaatcaggtgtgtaccatttgtactgagaacaccacagaatgaattatccaaagtccattgattttaatacgtgttttggtttgtaaacaaattatagtaatttcttgcacatttttggaaattaattgtataagaatttatgtatctgcttctagatacagtgtgtaaataaaaaatttcgttcacaaga

SEQ ID NO: 19 - protein, 645 aa, Homo sapiens
MQEAELRDGEAAAAAASYRVLSRLLGYGEAAPEPGPPPPPPGHGPPPPPFLARPGPRGSRPPQLMVFRNVGRPPEEEDVEAAPEPGPSELLCPRHRCALDPKALPPGLALERTWGPAAGLEAQLAALGLGQPAGPGVKTVGGGCCPCPCPPQPPPPQPQPPAAAPQAGEDPTETSDALLVLEGLESEAESLETNSCSEEELSSPGRGGGGGGRLLLQPPGPELPPVPFPLQDLVPLGRLSRGEQQQQQQQQPPPPPPPPGPLRPLAGPSRKGSFKIRLSRLFRTKSCNGGSGGGDGTGKRPSGELAASAASLTDMGGSAGRELDAGRKPKLTRTQSAFSPVSFSPLFTGETVSLVDVDISQRGLTSPHPPTPPPPPRRSLSLLDDISGTLPTSVLVAPMGSSLQSFPLPPPPPPHAPDAFPRIAPIRAAESLHSQPPQHLQCPLYRPDSSSFAASLRELEKCGWYWGPMNWEDAEMKLKGKPDGSFLVRDSSDPRYILSLSFRSQGITHHTRMEHYRGTFSLWCHPKFEDRCQSVVEFIKRAIMHSKNGKFLYFLRSRVPGLPPTPVQLLYPVSRFSNVKSLQHLCRFRIRQLVRIDHIPDLPLPKPLISYIRKFYYYDPQEEVYLSLKEAQLISKQKQEVEPST

SEQ ID NO: 20 - DNA, 8258 bp, Homo sapiens
ggcttgggggcggtgctcggcggtggcggagcgcggcctgggctcgcgctgggctccgcgcgccccccgcccccctctatgaggcagaggccgcggcggccgttagcgctgtcgctccgggggccgcggcgggcggggctccggcggggcccggcctagtccccaccccagcccggctcccagccgcccgccctccctccctctccccgatgcaggaggccgagctccgggatggcgaggcggcggcggcggccgcttcgtaccgcgtcctgagccgcctccttggctatggagaggcggcccccgagccaggccctccgccaccgcccccgggccatggccccccgccgccacccttcctcgcgcggcccggcccgcggggctcccggccgccgcagctgatggtgttccgcaacgtgggtcggccgccggaggaggaggacgtggaggcggccccggagccgggaccctcggaactgctgtgtccccggcaccgctgtgccctggaccccaaggccctgccgccgggcttggcgctcgagcggacctggggcccggcggctggactagaggcgcagttggcggctctggggctcgggcagccggcggggccgggggtcaagacagtcggtgggggttgctgcccgtgtccgtgtcctcctcagccgccccctccgcagccccagccgcctgctgccgccccgcaggccggggaggaccccacggaaacgagcgacgcgctgctggtcctggagggcttggaatcggaggccgagagcctggagactaacagctgctcggaagaggagctcagcagcccgggtcgcggaggaggagggggcggccggcttctgctgcagcccccaggccctgaattacctccggtgcccttcccgctgcaggacttggtccctctggggcgcctgagtagaggggagcagcagcagcagcagcagcagcaacctcccccgcccccgcctcctcccgggcccctccggccactcgcgggtccttctcggaagggctccttcaaaatccgcctcagtcgcctctttcgcaccaagagctgcaacggtggctccggcggtggggatgggaccggcaagaggccttctggagagctggctgcttcagctgcgagcctgacagacatgggaggctctgcgggccgggagctggacgcggggaggaaacccaagttgacaagaactcaaagtgccttttctccggtctccttcagccccctgttcacaggtgaaactgtgtcgcttgtggatgtggacatttctcagcggggcctgacctctccacaccctccaactccccctcctcctccgagaagaagcctcagcctcctagatgatatcagtgggacgctgcctacatctgtccttgtggctccgatggggtcttccttgcagtctttccccctacctccgcctcctccaccccatgccccagatgcatttccccggattgctcccatccgagcagctgaatccctgcacagccaacccccacagcacctccagtgtcccctctaccggcctgactcgagcagctttgcagccagccttcgagagttggagaagtgtggttggtattgggggccaatgaattgggaagatgcagagatgaagctgaaagggaaaccagatggttctttcctggtacgagacagttctgatcctcgttacatcctgagcctcagtttccgatcacagggtatcacccaccacactagaatggagcactacagaggaaccttcagcctgtggtgtcatcccaagtttgaggaccgctgtcaatctgttgtagagtttattaagagagccattatgcactccaagaatggaaagtttctctatttcttaagatccagggttccaggactgccaccaactcctgtccagctgctctatccagtgtcccgattcagcaatgtcaaatccctccagcacctttgcagattccggatacgacagctcgtcaggatagatcacatcccagatctcccactgcctaaacctctgatctcttatatccgaaagttctactactatgatcctcaggaagaggtatacctgtctctaaaggaagcgcagctcatttccaaacagaagcaagaggtggaaccctccacgtagcgaggggctccctgctggtcaccaccaagggcatttggttgccaagctccagctttgaagaaccaaattaagctaccatgaaaagaagaggaaaagtgagggaacaggaaggttgggattctctgtgcagagactttggttccccacgcagccctggggcttggaagaagcacatgaccgtactctgcgtggggctccacctcacacccacccctgggcatcttaggactggaggggctccttggaaaactggaagaagtctcaacactgtttctttttcagaagttttgtttttgatatttatattacttggtatggaaaactcaccttgaaggcagttggggtttgtgcccgttggattgaaagtggtgcgaagggtgagcaggtccaaagaaggggtgggaggagggaacaggggacggccattcagctggtgccaaaggcagagttagagtctgtgctgtgggcctggaagatgggaggaggggcttgaggtttgcaaaggactgggagttcctgaggaagggggaatctgcctcttgttgccatgggcagctgttgtaggagcaggagagaaaggagggtgggtggtctcgaaaagaatattgggcaaaacctagccaattggccttagctgggagaagtagtgactcctgcatccttttttaaggtttaggaacctgagttcagaaacacctctcatggaagctgtactagttgtgatttacttaattccttaagttccatgacctgaagttaaccccgttcttcctctgctctcaacccattgccccttgagataactgtacatgtcactctgatcatggtaacagcatccctattgcttctgccagctgtcatggcaatcgtgtttcccatcacctgggcggttcagagccagtcatgggctgctgaatttaatggagcatgtttccaggttcttcatggcaaactgtactcatgacttaggagtgagtgttacttccatgtgcctgtcagcttgtgagggggaatgtggaggaaggtgagaaatacagctcccacagttgtgctcttcctagaggaagctctcagaacgcagccctcacgggatttccttaggtcagaggagagcatcgcatctcacgtttttaggtttatcactgccatcccacttctgggatgggaggtagcaagggcttctgtattttcttgtgttcattctagcaacccagacatttccggatcagatcctgctggtctccactcactggaaagtctgccagatgccgatttgagagctgcctgtccctgctttcaggaggagcggggagaaaaactccaatggtctttaatggtttctgcagctggccatggccaattcatatgacattgtgagtttgctttcttatagagctgctctggggagaggtttgctattgagatgtaacagtggagctgttgggtcttcatgactcctttgcgtgtgttccatgggactctctttctgggttccccatgcttatagttgcctcgtgtcacaagacagatactaatgtcaggtttgtggcttcctgatggtttgggtggggccccagtgtcctggtaatttataggactgcctcatctgggagcattgccttcttccttagtcccacgtggagtgaccagtcttcctccttgtagctgaacagggaggaaacttgcaccattacctgactgtggaagggtggcccacaagatgagctgtgcaccataaacacagcccacctctgatttgtcatgtggtacctcttctttccttggcttccatggtagtattatcaactaagcaagattgtgatcccagaaattggcttagcatgtgagtgttgcctcgtgagagtacaagtaatataactcgccatcttgcaggaagtgccaccccaatatagagcctgaagttggaatctgttgagatccttgggtggctgatatacagcctgggatctttcttttttttgttccttttcaaccacccataattttaatattattttttagtgtgtgtgtgcctggctttgcgctagatattgtagaaaacaaaaaaggtaaaagacgtaatatgtggcctaagggagcttttaggtgactgctgcacatcaagcagaaaatcaaggactatctaaagacgtttatagtagataagatcagggtagaccagatggtctgggaaagttctgtgcctctgaggctttgggttgtagtcaatggcaggacagacagtgagatgaaaaacacatgagcaaaagcaaggaagcagaaatctgcatggcatgtactgaacagtgcacagccctgttagagcaacatggttaaagaatcctttccagtgcggttttctagatggaagcttcccagccaccaggcagacctgagtgccaaggggttatgatggtgaggtggagcccaaagcccaaaggagtcagcaaggctcctgcccattgccagggcctcactatggtcagctcaggccatgtgagggaggcagagcctctgcaccccctgtgttactggggtttcttctggagaactcatacattcaggtacaaaacaaaccaactgaggaggtgtgacccaacctcaccacccacctttctcctcctggggagtgtcgtgttgaactgtgtctgtgtcagtgcactggtcccagccctggccgcagcctagtcctttctctgtggagtgggctgcaaaacagcagcgcaagaggagacagtcttcgtcaggtgtgggtgtttggtgcctgcatgggtggccttgcacagagcagttaggggaagatgagggggaggcatggggctgggccagctctctgggatacagcctggtcagagagtaaatggagcagaggagcaggtgtggcagcacctgcccttcacctccctgaccaggcccgtccctccttcgccagtgctgtgcagagctcatttaaatgtattcctttctaggtctgggcgcggtggctcacgcctgtaatcccagcactttgggaggctgaggtgggtggatcatgaggtcgggagttcaagaccagcttggccaaacagtgaaaccctatctctactaaaatacaaaaattagctgggcatggtggcatgcgcctgtagtcccagctacttgggaggctgagacaggagaattgcttgaacccaggaggcggaggttgcactgagccgagattgcgccactgccctctagcctagacgacagagtgagactccatctcaaaaaaaataaataatttctttctggttgtattttcaagtcacaaattggaaaaggcttacatctagggacccactgttgattcctaagttgtgggtgggtgatatcagtggcctcagtgcctgcataaccctcacctgtttatgactgatctactgtaaccttcctcaggttgagagtgagctgctgtgagccagtgacaactactgctacaacttgctgttacttgaaattcgtgtgctatgttggtagcacaggagtaggcgggcgggggttttggtgtgggcactaggtaagtgggtggcaaccggctctttgggaccattggtgctgacctttgcctggtcacctgcctgtgcctaggccaagtgaatgttacatcttggaacctgagccatggaggtggcgccacctccctgaggagaaaggtgtggccaaaggaaactcctgcatggctcctgctccttccccttccctacatttcacttgggctaatgatgtaagccccccttgaggttggggatggtgtggagatagctgcagacacccctgtaacagacatccaggaagaattagggagtgggggtaattatagttcctaggtctaggggtgagaggtgggagtagaaccagaagtgccctggaatccagccgtgtcagccctcaccccagggcccccaggaaagcatggcctgcttgaagccctctgctccctgcagaaactggggcgaggaggagctttttccttcagttagatctttcccttccctgctgcctcgctcccttagctttggagccctaaagttagtggacaagacactgggataaagctctatggctgctggaaggaggtctgagtctgtgtgtgtgcatatgcatgcatgtgtgtgtgtgtgtgtgtcctcatttgcagaagtcttgctaccagttagggtgttaagagcatctccactgggcggagacctggcatttgttttccactgttaagagaatgacacaccccctgtcatgtaagggaggagctattgagttagacattttccctatgggaatcctcggtttggtttgtgggaaaggagggaatggataagtgatttttatctctaatcgtcaacacagctgttcttccactgaatttgtgctattgcatacatgtagccatctttcttttcactgcagcagtgtttatcagtagttcaaaatgatttatttgctcctggggagtaaaaccttttttattaaaaaaagaaaaagaaaaaaaaaagaaagaaaagtgtgcccccctcctatgctgcgatagctataaaatgattgggtcacaaaggtcaagtctgcacgtccctgtggtagaccctgctttcacattggagccttcacactcagcacaaaatgattggaggccttgataaaatggagagcaaagtcttgggagcagtgaaatgggggttggatcatagagacaggcgctggggactcgggtactgcagttaggaaagtagcgttatgagttgtactgaaaatgttgattctctaatctgccagaaaaggacctgtcttttcatgcagatttcatattgtctttgtccttttcattgcttcttgaccttcctggcaggtgtcgctcagtttcttcctgtttcccttcctgtcctctccacacctgctatcccgtcccactcccatctacctcccgggaagccagccctgcatgctgagtttgtgacctgcttcattcccatttcatttctagagggtttagaggtgacctggaaccgttccctttccctctcctaccccctcctctgcaacaccaagaggcctggaggggcagacagaaagcagccagccacggcgggaagacatgcatgtttggttgcagctggactgcgatcgtagttcctcctggagatagagtgtgaggaacttaggacactcttcctcagactctgggatcatcacataccacactgccccgctcagagtttcgtcctgagctccctaaccagctcaggtggagcagaagcctgctctcactcctccatctctggtgctcccttgggcggggacctgtccctcactcttaggcccagaacctgtccaagggacaggtagggtccaggtgccactttgggtagctggctgttggaatgcccacactggtgctgcctgtggcatagccactgctgtacgtttttggttgtttttaagaaactcgatgaagaggggtgtcattctgggctcggggtggttgccaatttttcaccagaaagggagccaccccttgcaaccacttctgtctccgttagccccccctctgccctcctccaagccaaagcgtggcctggcttttgtcttcccatttagttttcctcttttacccttccttttgtgcttaatttattaaaatagttgctgtataatttattttcataaactataaaaaaatactaaatggttaaaatagacttgcaggccaatcttaaatggggtgggaggggtctgagggtgggatggggaaagggaaagaggttttgatataaacaaaacaaatgcactttgggtgtgttttggtatttttctggggatagagggggtggggttagggatgtccctgtagattagttccagaatggggtgtctgtatatactgtattaataggcatgtttgactctcgtaaagggacgttagtagctgctgcaggtcctgtttggaaaccccatgtacaattcccagttttttgtaagtgtcagtgcgagagacatttgactcttgtgtttgtatctcctttttatgattgctgtacctacccatgtctttttggggaggggtgaaaagagatttgaaataaaaatgtttagaaatta

SEQ ID NO: 21 - RNA, 22 nt, Mus musculus
cauugcacuugucucggucuga

SEQ ID NO: 22 - RNA, 22 nt, Artificial Sequence
  mir mimic
cauugcacuugucucggucuga

SEQ ID NO: 23 - RNA, 20 nt, Mus musculus
uaaggcacgcggugaaugcc

SEQ ID NO: 24 - RNA, 20 nt, Artificial Sequence
  mir mimic
uaaggcacgcggugaaugcc

SEQ ID NO: 25 - RNA, 22 nt, Mus musculus
ugagguaguagguuguauaguu

SEQ ID NO: 26 - RNA, 22 nt, Mus musculus
ugagguaguagguuguaugguu

SEQ ID NO: 27 - RNA, 22 nt, Artificial Sequence
  Synthetic antagomir
ugagguaguagguuguauaguu

SEQ ID NO: 28 - RNA, 22 nt, Artificial Sequence
  Synthetic antagomir
ugagguaguagguuguaugguu

SEQ ID NO: 29 - DNA, 20 bp, Artificial Sequence
  Synthetic primer
ggcattgctctcaatgacaa

SEQ ID NO: 30 - DNA, 20 bp, Artificial Sequence
  Synthetic primer
cttgctcagtgtccttgctg

SEQ ID NO: 31 - DNA, 24 bp, Artificial Sequence
  Synthetic primer
tacgacatgaacggctgctactca

SEQ ID NO: 32 - DNA, 24 bp, Artificial Sequence
  Synthetic primer
ttacatgctgcaggatctccacct

SEQ ID NO: 33 - DNA, 8 bp, Artificial Sequence
  Motif
vcasctgm

SEQ ID NO: 34 - DNA, 12 bp, Artificial Sequence
  Motif
thvdbasaavrw

SEQ ID NO: 35 - DNA, 10 bp, Artificial Sequence
  Motif
  misc_feature (1)..(1): n is a, c, g, or t
nwkscsabad

SEQ ID NO: 36 - RNA, 29 nt, Artificial Sequence
  Predicted miRNA sites in Dkk3
ccagcugagucacugguagaugugcaaua

SEQ ID NO: 37 - RNA, 22 nt, Artificial Sequence
  predicted miR-25 sites
agucuggcucuguucacguuac

SEQ ID NO: 38 - RNA, 29 nt, Artificial Sequence
  predicted miR-25 sites
gauacuguaacaaaacggcugaagcaaug

SEQ ID NO: 39 - RNA, 22 nt, Artificial Sequence
  predicted miR-25 sites
agucuggcucuguucacguuac

SEQ ID NO: 40 - RNA, 29 nt, Artificial Sequence
  predicted miR-25 sites
cuuucuggcuaacaggagagugggcaaug

SEQ ID NO: 41 - RNA, 22 nt, Artificial Sequence
  predicted miR-25 sites
agucuggcucuguucacguuac

SEQ ID NO: 42 - RNA, 29 nt, Artificial Sequence
  let-7 sites in Klf4 3′UTR
cggaggaagaggaagcgauucagguacag

SEQ ID NO: 43 - RNA, 22 nt, Artificial Sequence
  let-7 sites in Klf4 3′UTR
ccuuucgaucuuccaacauguc

SEQ ID NO: 44 - RNA, 29 nt, Artificial Sequence
  let-7 sites in Klf4 3′UTR
ugguuucagaugugcaauaauuuguacaa

SEQ ID NO: 45 - RNA, 19 nt, Artificial Sequence
  let-7 sites in Klf4 3′UTR
uucgaucuuccaacauguc